SEQUENCE LISTING (29 sequences)

SEQ ID NO: 1 - DNA, 22 bp, Artificial
  c-Myc primer
acctaactcgaggaggagctgg

SEQ ID NO: 2 - DNA, 21 bp, Artificial
  c-Myc primer
tccacatagcgtaaaaggagc

SEQ ID NO: 3 - DNA, 20 bp, Artificial
  Klf4 primer
acactgtcttcccacgaggg

SEQ ID NO: 4 - DNA, 21 bp, Artificial
  Klf4 primer
ggcattaaagcagcgtatcca

SEQ ID NO: 5 - DNA, 19 bp, Artificial
  Sox2 primer
cattaacggcacactgccc

SEQ ID NO: 6 - DNA, 20 bp, Artificial
  Sox2 primer
cgcccggagtctagctctaa

SEQ ID NO: 7 - DNA, 21 bp, Artificial
  Oct4 primer
agcctggcctgtctgtcactc

SEQ ID NO: 8 - DNA, 21 bp, Artificial
  Oct4 primer
ggcattaaagcagcgtatcca

SEQ ID NO: 9 - DNA, 20 bp, Artificial
  GAPDH primer
ttcaccaccatggagaaggc

SEQ ID NO: 10 - DNA, 18 bp, Artificial
  GAPDH primer
cccttttggctccaccct

SEQ ID NO: 11 - DNA, 21 bp, Artificial
  mSox2 primer
gcagggagttcgcaaaagtct

SEQ ID NO: 12 - DNA, 21 bp, Artificial
  mSox2 primer
ggcattaaagcagcgtatcca

SEQ ID NO: 13 - DNA, 19 bp, Artificial
  mOct4 primer
acatcgccaatcagcttgg

SEQ ID NO: 14 - DNA, 23 bp, Artificial
  mOct4 primer
agaaccatactcgaaccacatcc

SEQ ID NO: 15 - DNA, 22 bp, Artificial
  mNanog F primer
cctccagcagatgcaagaactc

SEQ ID NO: 16 - DNA, 23 bp, Artificial
  mNanog F primer
cttcaaccactggtttttctgcc

SEQ ID NO: 17 - DNA, 21 bp, Artificial
  mRex1 F primer
tgtccccaaataccactgacc

SEQ ID NO: 18 - DNA, 19 bp, Artificial
  mRex1 F primer
ggcactgatccgcaaacac

SEQ ID NO: 19 - DNA, 21 bp, Artificial
  mKlf4 F primer
tgcttggtgagttgtggtcct

SEQ ID NO: 20 - DNA, 19 bp, Artificial
  mKlf4 F primer
agaactttggctcccccgt

SEQ ID NO: 21 - DNA, 19 bp, Artificial
  c-Myc F primer
acatcgccaatcagcttgg

SEQ ID NO: 22 - DNA, 23 bp, Artificial
  c-Myc F primer
agaaccatactcgaaccacatcc

SEQ ID NO: 23 - DNA, 28 bp, Artificial
  ubiquitin promoter primer detecting
gagtcttgaatggaagacgcttgtgagg

SEQ ID NO: 24 - DNA, 22 bp, Artificial
  c-Myc primer detecting
actgaggggtcaatgcactcgg

SEQ ID NO: 25 - DNA, 22 bp, Artificial
  Klf4 primer detecting
cctggtgggttagcgagttgga

SEQ ID NO: 26 - DNA, 30 bp, Artificial
  hOcCT4PE KpnI primer
ggtaccggatactcaggccaggcccagaaa

SEQ ID NO: 27 - DNA, 25 bp, Artificial
  hOCT4PE XhoI primer
ctcgagtccacagacctctggcact

SEQ ID NO: 28 - DNA, 35 bp, Artificial
  hOCT4DE KpnI primer
ggtacccattgagtccaaatcctctttactaggtg

SEQ ID NO: 29 - DNA, 25 bp, Artificial
  hOCT4DE XhoI primer
ctcgagctgaggctcatgctgctgg

(19) United States
(12) Patent Application Publication
(10) Pub. No.: US 2011/0088107 A1
(43) Pub. Date: Apr. 14, 2011

(54) COMPOSITIONS AND METHODS FOR DERIVING OR CULTURING PLURIPOTENT CELLS

(76) Inventors: Yaqub Hanna, Boston, MA (US); Rudolf Jaenisch, Brookline, MA (US)

(21) Appl. No.: 12/767,606

(22) Filed: Apr. 26, 2010

Related U.S. Application Data
(60) Provisional application No. 61/214,502, filed on Apr. 24, 2009.
(60) Provisional application No. 61/323,830, filed on Apr. 13, 2010.

(51) Int. Cl. C12N 15/873; C12N 5/073

(52) U.S. Cl. 800/21; 435/325; 435/375; 435/377

(57) ABSTRACT

The invention provides compositions and methods useful for deriving or culturing vertebrate ES cells. Certain inventive methods comprise deriving or culturing vertebrate ES cells using medium that comprises a compound that replaces Klf4 or c-Myc in generating iPS cells. The invention provides NOD ES cells and methods of deriving or culturing them.

### RELATED APPLICATIONS

This application claims the benefit of U.S. Provisional Application Ser. No. 60/214,502, filed Apr. 24, 2009, and U.S. Provisional Application Ser. No. 61/323,830, filed Apr. 13, 2010, the entire teachings of which are incorporated herein by reference.

### GOVERNMENT FUNDING STATEMENT

The United States Government has provided grant support utilized in the development of the present invention. In particular, NIH grants 5-RO1-HDO045022, 5-R37-CA084198, and 5-RO1-CA087869 have supported development of this invention. The U.S. Government has certain rights in the invention.

### BACKGROUND OF THE INVENTION

Embryonic stem (ES) cells are of great scientific and practical interest. For example, mouse ES cells are widely employed to engineer precise modifications to the genome by gene targeting. Human ES cells hold great promise for regenerative medicine applications. ES cells were first derived from mice and have subsequently been derived from a number of other species, including humans and non-human primates. The efficiency of ES cell derivation, e.g., in mice, is affected by genetic background. In addition, ES cells of different species vary in terms of the ease with which they can be derived and maintained. Human ES cells can be technically demanding to culture and exhibit properties such as slow growth and poor tolerance to passaging as single cells.

### SUMMARY OF THE INVENTION

The present invention provides methods of deriving or culturing pluripotent vertebrate cells. The invention also provides non-human vertebrates, e.g., non-human mammals, e.g., mice, derived from the pluripotent cells.

In one aspect, the invention provides a non-obese diabetic (NOD) embryonic stem (ES) cell. In some embodiments the NOD ES cell is derived from a cell obtained from the inner cell mass (ICM) of a blastocyst of a NOD mouse. In some embodiments the NOD ES cell is derived from a cell obtained from the epiblast of a NOD mouse embryo. In some embodiments the cell does not persistently, e.g., constitutively, express Klf4 or c-Myc. In some embodiments the ES cell is not genetically modified. In some embodiments the ES cell is germline-competent.

In another aspect the invention provides chimeric mouse derived in part from a germline-competent NOD ES cell. In some embodiments at least some germ cells of the chimeric mouse are descendants of the ES cell. The invention also provides a NOD mouse derived from a NOD ES cell. In some embodiments the mouse is produced without production of a chimera.

In some embodiments the NOD ES cell has at least one genetic modification of interest. The invention also provides a chimeric mouse derived at least in part from a genetically modified NOD ES cell. In some embodiments at least some germ cells of the mouse are descendants of the ES cell. The invention also provides a genetically modified NOD mouse derived from a genetically modified NOD ES cell.

In another aspect, the invention provides a composition comprising (i) a cell obtained from the ICM of a NOD mouse blastocyst or obtained from the epiblast of a NOD mouse embryo; and (ii) a compound that replaces Klf4 or c-Myc in generating iPS cells. In some embodiments the compound is a Wnt or a GSK3 inhibitor. In some embodiments the GSK3 inhibitor is CHIR99021 or a structurally related compound. In some embodiments the compound is a paullone. In some embodiments the composition comprises a GSK3 inhibitor and a paullone. In some embodiments the GSK3 inhibitor is CHIR99021 or a structurally related compound.

In another aspect, the invention provides a method of producing a chimeric mouse that comprises cells derived from a NOD mouse, the method comprising steps of: (a) providing a NOD ES cell; (b) introducing the NOD ES cell into a mouse blastocyst; (c) transferring the blastocyst to a pseudopregnant female mouse; and (d) maintaining the female mouse under conditions suitable for production of live offspring. Other methods for producing a chimeric mouse are within the scope of the invention.

In another aspect, the invention provides a method of producing a NOD mouse comprising steps of: (a) providing male and female chimeric mouse produced according to the method of claim 19, wherein at least some germ cells of the chimeric mice are descendants of the NOD ES cell; and (b) interbreeding the chimeric mice of step (a).

The invention also provides a cell colony composed of NOD ES cells.

The invention also provides a karyotypically normal NOD ES cell line that remains stable over at least 20 passages.

In another aspect, the invention provides a vertebrate cell isolated from the ICM of a vertebrate blastocyst or from the epiblast of a vertebrate embryo, wherein the vertebrate cell has been caused to express Klf4 or c-Myc. In some embodiments the cell has been caused to express Klf4 or c-Myc by introducing into the cell (or an ancestor of the cell), a nucleic acid construct that encodes Klf4 or c-Myc, operably linked to a promoter functional in the cell. In some embodiments the promoter is constitutive. In some embodiments the promoter is inducible. In some embodiments, the vertebrate cell is a mouse cell. In some embodiments, the vertebrate cell is a primate cell. In some embodiments, the vertebrate cell is a human cell and is isolated from the ICM of a human blastocyst.

In another aspect the invention provides a composition comprising: (a) a vertebrate cell isolated from the ICM of a vertebrate blastocyst or isolated from the epiblast of a vertebrate embryo; and (b) a compound that replaces Klf4 or c-Myc expression in generating iPS cells. In some embodiments, the compound is a Wnt or is a GSK3 inhibitor. In some embodiments, the compound is a paullone. In some embodiments, the composition comprises a GSK3 inhibitor and a paullone. In some embodiments, the GSK3 inhibitor is CHIR99021 or a structurally related compound. In some embodiments, the cell is of a strain or species that is not otherwise permissive for deriving ES cells. In some embodiments, the vertebrate cell is a rodent cell. In some embodiments, the vertebrate cell is a primate cell. In some embodiments, the cell is a human cell isolated from the ICM of a human blastocyst. In another aspect the invention provides a composition comprising: (a) a vertebrate ES cell; and (b) a compound that replaces Klf4 or c-Myc expression in generating iPS cells. In some embodiments, the compound is a Wnt or is a GSK3 inhibitor. In some embodiments, the compound is a paullone. In some embodiments, the composition comprises a GSK3 inhibitor and a paullone. In some embodiments, the GSK3 inhibitor is CHIR99021 or a structurally related compound. In some embodiments the cell is of a strain or species that is not otherwise permissive for deriving ES cells. In some embodiments, the vertebrate cell is a rodent cell. In some embodiments, the vertebrate cell is a primate cell. In some embodiments, the ES cell has at least one improved property relative to an ES cell of the same strain or species not cultured in medium containing the compound. In some embodiments, the improved property comprises increased tolerance to dissociation. In some embodiments, the improved property comprises increased amenability to genetic modification.

In another aspect, the invention provides a method of deriving a vertebrate ES cell, the method comprising: (a) providing a vertebrate cell isolated from the ICM of a vertebrate blastocyst or isolated from the epiblast of a vertebrate embryo; (b) causing the cell to persistently express Klf4 or c-Myc or culturing the cell in medium that contains a compound that replaces Klf4 or c-Myc expression in generating iPS cells; and (c) maintaining the cell in culture under conditions suitable and a time sufficient to produce a vertebrate ES cell. In some embodiments, the compound is a Wnt or a GSK3 inhibitor. In some embodiments, the compound is a paullone. In some embodiments, the composition comprises a GSK3 inhibitor and a paullone. In some embodiments, the e GSK3 inhibitor is CHIR99021 or a structurally related compound. In some embodiments, the ES cell is germline-competent. In some embodiments, the vertebrate is of a strain or species that is non-permissive for deriving ES cells. In some embodiments, the vertebrate is of a strain that is non-permissive for deriving ES cells and the wherein the conditions in step (c) are conditions suitable for deriving ES cells from a blastocyst of a vertebrate of the same species but of a different strain that is permissive for deriving ES cells. In some embodiments, the vertebrate cell is a mouse cell. In some embodiments, the vertebrate cell is a primate cell. In some embodiments, the vertebrate cell is a NOD mouse cell. In some embodiments, the vertebrate cell is a human cell isolated from a human blastocyst. In another aspect, the invention provides a method of culturing a vertebrate ES cell, the method comprising: (a) providing a vertebrate ES cell; and (b) culturing the cell in medium that contains a compound that replaces Klf4 or c-Myc expression in generating iPS cells. In some embodiments, the compound is a Wnt or a GSK3 inhibitor. In some embodiments, the compound is a paullone. In some embodiments, the composition comprises a GSK3 inhibitor and a paullone. In some embodiments, the GSK3 inhibitor is CHIR99021 or a structurally related compound. In some embodiments, the ES cell is a mouse ES cell. In some embodiments, the ES cell is a NOD mouse ES cell. In some embodiments, the ES cell is a primate ES cell. In some embodiments, the ES cell is a human ES cell.

In another aspect, the invention provides a composition comprising an induced pluripotent stem (iPS) cell and a compound that replaces Klf4 or c-Myc expression in generating iPS cells, wherein said iPS cell is unstable in the absence of the compound. In some embodiments, the iPS cell is a mouse cell. In some embodiments, the iPS cell is a NOD mouse cell. In some embodiments, the e cell is not genetically modified. In some embodiments, the compound is a Wnt or is a GSK3 inhibitor. In some embodiments, the compound is a paullone. In some embodiments, the composition comprises a GSK3 inhibitor and a paullone. In some embodiments, the GSK3 inhibitor is CHIR99021 or a structurally related compound.

In another aspect, the invention provides an induced pluripotent stem (iPS) cell derived from a somatic cell of a NOD mouse. In some embodiments, the cell does not constitutively express Klf4 or c-Myc. In some embodiments, the cell is not genetically modified. In another aspect, the invention provides a composition comprising an iPS cell derived from a somatic cell of a NOD mouse and a compound that replaces Klf4 or c-Myc expression in generating iPS cells. In some embodiments, the compound is a Wnt or is a GSK3 inhibitor. In some embodiments, the compound is a paullone. In some embodiments, the e composition comprises a GSK3 inhibitor and a paullone. In some embodiments, the GSK3 inhibitor is CHIR99021 or a structurally related compound. In some embodiments, the iPS cell is germline-competent.

In another aspect, the invention provides a chimeric mouse derived at least in part from an iPS cell derived from a somatic cell of a NOD mouse, wherein at least some germ cells of the mouse are descendants of the iPS cell. In another embodiment the invention provides a NOD mouse obtained by interbreeding male and female of the foregoing chimeric mice. In another embodiment the invention provides a mouse obtained by interbreeding a chimeric mouse derived at least in part from a NOD ES cell or derived at least in part from a NOD iPS cell with a mouse of a strain of interest.

The invention further provides NOD mouse derived from a NOD iPS cell. In some embodiments, the mouse is derived without production of a chimera.

The invention also provides a NOD iPS cell, wherein the cell has a genetic modification of interest. The invention also provides a genetically modified NOD mouse derived from said NOD iPS cell.

In another aspect, the invention provides a method of deriving an iPS cell from a somatic cell of a NOD mouse comprising steps of: (a) providing a somatic cell obtained from a NOD mouse; (b) causing the cell to express a set of reprogramming factors or contacting the cell with a set of reprogramming compounds sufficient to induce the cell to become reprogrammed to a pluripotent state; and (c) causing the cell to persistently express Klf4 or c-Myc or culturing the cell in medium that contains a compound that replaces Klf4 or c-Myc expression in generating iPS cells. Suitable compounds are as described for other methods herein.

In another aspect, the invention provides a method of producing a chimeric mouse that comprises cells derived from a NOD mouse, the method comprising steps of: (a) providing an iPS cell derived from a somatic cell of a NOD mouse; (b) introducing the iPS cell into a mouse blastocyst; (c) transferring the blastocyst to a pseudopregnant female mouse; and (d) maintaining the female mouse under conditions suitable for production of live offspring. Suitable compounds are as described for other methods herein.

In another aspect, the invention provides a method of producing a NOD mouse comprising steps of: (a) providing male and female chimeric mice produced according to the method of claim 91, wherein at least some germ cells of the chimeric mice are descendants of the iPS cell; and (b) interbreeding the chimeric mice of step (a).

In some aspects, the invention provides improvements in methods and compositions for deriving or culturing pluripotent vertebrate cells.

In another aspect, the invention relates to a method of modifying the pluripotency state of a vertebrate cell to a more naïve state, the method comprising: (a) providing a pluripotent vertebrate cell; (b) causing the cell to express Klf4 and/or Klf2; (c) culturing the cell in the presence of a GSK3 inhibitor, a mitogen-activated protein kinase pathway inhibitor, and an activator of the Stat3 pathway; and (d) maintaining the cell in culture under conditions suitable and a time sufficient to produce a vertebrate cell having a resulting pluripotency state which is more naïve than the pluripotency state of the vertebrate cell of step (a). The invention also relates to a method of culturing a vertebrate cell, the method comprising: (a) providing a pluripotent vertebrate cell; (b) causing the cell to express Klf4 and/or Klf4; and (c) culturing the cell in the presence of a GSK3 inhibitor, a mitogen-activated protein kinase pathway inhibitor, and an activator of the Stat3 pathway.

In some embodiments step (b) of the method further comprises causing the cell to express Oct4; in particular embodiments the method comprises upregulating expression of or overexpressing one or more of Oct4, Klf4 and Klf4. In some embodiments expression of one or more of Oct4, Klf4 and Klf2 is achieved via expression of one or more ectopic transgenes. In other aspects methods of upregulating expression of one or more of Oct4, Klf4 and Klf2 include protein transfection methods and methods of inducing pathways which produce upregulation of one or more of Oct4, Klf4 and Klf2. In some aspects causing the cell to express Klf4 and/or Klf2 is achieved by culturing the cell in the presence of a compound which induces Klf4 and/or Klf2 expression. In particular embodiments the compound is a protein kinase A pathway agonist, e.g., forskolin. In particular embodiments the GSK3 inhibitor is CHIR99021 or a structurally related compound. In particular embodiments the mitogen-activated protein kinase pathway inhibitor is PD0325901. In certain embodiments the activator of the Stat3 pathway is LIF.

In some aspects the cell is a mammalian cell, e.g., a primate, mouse, rabbit, rodent, human or other animal cell. In some aspects the cell is a non-permissive cell. In some aspects the cell is an induced pluripotent cell (iPS cell) such as an induced pluripotent stem cell (iPSC), e.g., a human induced pluripotent stem cell (hiPSC), or a human embryonic stem cell (hESC). In some aspects the cell is derived from a human embryonic stem cell line. In some aspects the cell is derived from an embryo, e.g., from the ICM or epiblast of said embryo.

In some aspects the cell is cultured in the absence of an activator of bFGF and/or Activin signaling; in some aspects the cell is cultured in the presence of an inhibitor of bFGF and/or Activin signaling.

In one aspect the resulting pluripotency state is stable as compared with the pluripotency state of the vertebrate cell of step (a). In one aspect the resulting cell exhibits at least one property which is similar to the corresponding property of mouse embryonic stem cells, wherein said property is selected from the group consisting of growth properties, X chromosome activation state, gene expression profile, cell signaling properties, and signaling pathway dependence. In one aspect the resulting pluripotency state comprises pre-X inactivation of the X chromosome.

In other aspects the resulting cell has a global gene expression profile which clusters with naïve mouse ESCs as opposed to mouse EpiSCs and/or less naïve human ESCs. In some aspects the resulting cell can be propagated and/or do not lose pluripotency in the presence of one or more of BMP4; inhibitors of the TGFβ pathway (e.g., A83-01, SB432151); and inhibitors of the FGF pathway. In other aspects the resulting cell retains an X chromosome in a pre-X inactivation state, wherein said X chromosome is inactivated upon differentiation of the cell into a somatic cell or into a less naïve cell (e.g., a conventional bFGF- and Activin-dependent hESC). In other aspects the resulting cell exhibits enhanced utilization of the distal Oct4 enhancer. In still other aspects the resulting cell is dependent on LIF signaling (e.g., via Stat3-dependent and -independent pathways).

The invention also relates to stable naïve embryonic stem cell derived from a non-permissive cell as described herein; stable naïve embryonic stem cells can be produced by the methods described herein. In certain embodiments the naïve embryonic stem cell exhibits at least one property which is similar to the corresponding property of mouse embryonic stem cells (e.g, naïve mouse ESCs or NOD ICM-like ESCs), wherein said property is selected from the group consisting of growth properties, X chromosome activation state, gene expression profile, cell signaling properties, and signaling pathway dependence. In certain aspects the resulting cells can be anzymatically passaged (as compared with mechanically passaged. In other aspects the resulting cells may be passaged more than 15, more than 20, more than 25, more than 35 or more than 50 times without differentiating and/or losing their pluripotency.

The invention further relates to a composition comprising (a) a cell expressing (e.g., induced to express or overexpressing) Oct4, Klf4 and/or Klf2, and (b) a GSK3 inhibitor, a mitogen-activated protein kinase pathway inhibitor, and an activator of the Stat3 pathway. In some embodiments the GSK3 inhibitor is CHIR99021 or a structurally related compound. In other embodiments the mitogen-activated protein kinase pathway inhibitor is PD0325901. In some embodiments the activator of the Stat3 pathway is LIF. In some aspects of the composition the GSK3 inhibitor, a mitogen-activated protein kinase pathway inhibitor, and an activator of the Stat3 pathway are contained in cell culture medium.

In other aspects the invention relates to a composition comprising (a) a pluripotent vertebrate cell, and (b) a GSK3 inhibitor, a mitogen-activated protein kinase pathway inhibitor, an activator of the Stat3 pathway, and a compound which induces Klf4 and/or Klf2 expression. In some embodiments the GSK3 inhibitor is CHIR99021 or a structurally related compound. In other embodiments the mitogen-activated protein kinase pathway inhibitor is PD0325901. In some aspects the activator of the Stat3 pathway is LIF. In particular embodiments the compound which induces Klf4 and/or Klf2 expression is a protein kinase A pathway agonist (e.g., forskolin) or a CREB activator. In other aspects the compound which induces Klf4 and/or Klf2 expression is a compound which elevates intracellular cAMP.

For example, compounds that either enhance cAMP synthesis or inhibit its metabolism elevate intracellular cAMP. cAMP is synthesized by adenylate cyclase, which is stimulated by the G protein Gs. Therefore activators of Gs may be of use. For example, cholera toxin elevates cAMP by ADP-ribosylating the alpha subunit of Gs, which causes Gs to be constitutively active and in turn activate adenylate cyclase. In addition, β-adrenergic receptors couple to and activate Gs, so β-adrenergic receptor agonists, e.g., β1-adrenergic receptor agonists may be of use. Numerous β-adrenergic receptor agonists are known in the art. Examples include isoproterenol, formoterol, and salmeterol. Furthermore, cAMP is metabolized by phosphodiesterases (PDEs), e.g., PDE4. PDE inhibitors, e.g., xanthine derivatives such as the methlated xanthines isobutylmethylxanthine, theophylline, caffeine, may be of use. Many additional PDE inhibitors are known in the art. For example, there has been considerable interest in PDE4, inhibitors for treatment of inflammatory conditions, among others, and numerous small molecule PDE4 inhibitors are known. cAMP analogs (e.g., dibutyryl cAMP, 8-bromo-cAMP) may also be of use.

The invention also relates to a cell colony composed of stable naïve embryonic stem cells derived from a non-permissive cell, e.g., a human cell. Such cells can be produced by methods according to the invention.

The invention also relates to a pluripotent vertebrate cell which has been caused to express (e.g., upregulation of expression of) KLf4 and/or Klf2 and in which the GSK3 pathway has been inhibited, the mitogen-activated protein kinase pathway has been inhibited, and the Stat3 pathway has been activated. In certain embodiments the cell is a human cell. In certain embodiments the cell is germline-competent.

The invention also relates to a differentiated cell or a vertebrate animal derived from the cells described herein (e.g., naïve vertebrate ESCs).

The invention also relates to a culture medium comprising a GSK3 inhibitor, a mitogen-activated protein kinase pathway inhibitor, an activator of the Stat3 pathway, and a compound which causes expression of Klf4 and/or Klf2 in a pluripotent cell cultured in the culture medium. In some embodiments the compound which causes expression of Klf4 and/or Klf2 causes overexpression of Klf4 and/or Klf2.

The invention also relates to methods of assaying for or screening for one or more compounds which are able to substitute for the expression (e.g., induction of expression) of one or more of Oct4, Klf4 and Klf2 in methods of producing vertebrate pluripotent cells having a more naïve pluripotency state than that of a starting pluripotent cell.

Certain conventional techniques of cell biology, cell culture, molecular biology, microbiology, recombinant nucleic acid (e.g., DNA) technology, immunology, etc., which are within the skill of the art, may be of use in aspects of the invention. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., editions as of 2008; Sambrook, Russell, and Sambrook, Molecular Cloning: A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Burns, R., Immunochemical Protocols (Methods in Molecular Biology) Humana Press; 3rd ed., 2005. All patents, patent applications, and other publications mentioned herein are incorporated herein by reference in their entirety.

### BRIEF DESCRIPTION OF THE DRAWINGS

FIGS. 1(A)-1(H) show generation of transgene-dependent NOD iPS cells. (A) Definitions and terms of reagents and cells used in this study. (B) Strategy used for reprogramming NOD or 129 MEFs into iPS cells by infection with Dox-inducible lentiviruses (TetO) encoding O,S,K,M. Representative images of colonies observed at different stages in the as indicated in the panels (P—passage number). (C) Colonies originally isolated at day 16 after infection from 3 independent experiments from NOD and 129 MEF cultures; only colonies that stained positive for Nanog at day 45 while being maintained with Dox were analyzed. Right column lists the number of iPS lines that remained pluripotent (Nanog and Oct4 positive) without Dox. (D) Immunostaining for pluripotency markers of NOD iPS subclone #1 maintained on Dox. AP: Alkaline Phosphatase. (E) Methylation analysis of Oct4 and Nanog promoters. Open squares indicate unmethylated and filled squares methylated CpG dinucleotides. Shown are 6 representative sequenced clones from NOD MEFs, V6.5 ES cells and three different Dox-dependent NOD iPS cell lines. (F) Quantitative RT-PCR analysis specific for the reactivation of the endogenous indicated pluripotency-related genes in Dox dependent NOD iPS lines. V6.5 ESCs, iPS cells grown in the absence of Dox and MEFs are used as controls. Relative expression levels were normalized to the average expression of control ES line V6.5. (G) Teratoma derived from Dox-dependent NOD-iPS#6 cell line. (H) Chimeric mice derived from Dox-dependent NOD-iPS cells. Chimerism is indicated by the albino and brown coat colors.

FIGS. 2(A)-2(G) demonstrate that induced pluripotency on NOD background is stabilized by constitutive ectopic expression of Klf4 or c-Myc. (A) Experimental plan for deriving Dox-independent NOD iPS lines. Constitutive lentiviruses driven by the ubiquitin promoter (abbreviated as Ubi) encoding different transcription factors were used to transduce Dox dependent NOD iPS cells followed by Dox withdrawal. (B) Images of cultures depicting colony growth in the absence of Dox after infection of NOD iPS cells with constitutive lentiviruses encoding the indicated factors. (C) RT-PCR for detection of transgene specific expression of O, S, K, M in NOD-iPS#1 cells infected with c-Myc (M) or Klf4 (K) constitutive lentivirus that were maintained independent of Dox. V6.5 ES cells and parental uninfected pluripotent (on Dox) or differentiated (40 hours after Dox withdrawal) NOD iPS cells in the presence or absence of Dox were used as controls. (D) PCR analysis for the detection of Ubi-c-Myc and Ubi-Klf4 proviruses in genomic DNA of subcloned infected NOD iPS lines. (E) Immunostaining for pluripotency markers of Dox-independent NOD iPS lines that had been infected with Ubi-Klf4 or Ubi-c-Myc lentiviruses. (F) Teratomas derived from NOD iPS#1 infected subclones (G) Summary of infections performed on mouse NOD and 129Sv/Jae MEFs and Rat TTFs with reprogramming factors transduced either by Moloney retroviral backbone vectors (pLib), by constitutively expressed lentiviral vectors (Ubi), or by Dox inducible lentiviral vectors (TetO). Experiments using the three different infection protocols were performed side by side. The ability to derive iPS cells was defined by the detection of Nanog positive clones that were developmentally pluripotent as tested by teratoma formation (129 and NOD cell lines) or EB formation (Rat iPS cell lines) and is indicated by ‘+’. NA: Not applicable.

FIGS. 3(A)-3(I) show generation of Klf4 and c-Myc transgenic NOD ES lines. (A) Summary of ESC derivation efficiency from NOD and permissive strain control blastocysts. (B) Representative images for NOD blastocyst at embryonic day 3.5 and ICM outgrowths after plating the blastocysts on feeder cells. The cells were infected with the indicated viruses and passaged in mESM. (C) Representative images of NOD colonies after infection with c-Myc and Klf4 viruses at passage two (P2) and stable established NOD ES lines at P5. NOD ES 1M was derived after infections with Ubi-c-Myc and three lines were obtained following Ubi-Klf4 infection (NOD ES 2K is shown). (D) Transgene expression by RT-PCR in various NOD ES lines. MEFs infected with O, S, K, M viruses were used as positive controls. (E) Southern analysis of c-Myc and Klf4 viral integrations in representative NOD ES cell lines derived following viral infection. NOD MEFs are used as background controls. (F-G) Karyotype and cell cycle analysis of transgenic NOD ES lines. (H) Immuno-fluorescent staining of NOD ES lines for pluripotency markers. (I) NOD ES cell derived chimeras from NOD ES 1M and 2K cell lines were mated with NOD mice with albino offspring indicating germline transmission.

FIGS. 4(A)-4(H) illustrate generation of genetically unmodified germline competent NOD ES cells. (A) Experimental outline for derivation of iPS lines that grow in the presence of small molecules and in the absence of Dox. Representative images of NOD iPS lines growing in mESM supplemented with the indicated small molecules or growth factors. (B) Single-cell cloning efficiency of NOD iPS cells in different growth conditions. WT or NOD-iPS cells grown in mESM and KP were sorted in 96 well plates and cultured in mESM supplemented with small molecules. The number of wells containing Nanog+ colonies was counted after 6 days. Efficiencies were normalized to that of C57B6/129F1-iPS cells (“permissive” controls) plated in mESM and defined as 100%. SD for average efficiencies from 2 experiments are shown. Student test P value compares KP/CH to PD/CH mESM conditions for NOD iPS cells. (C) Expansion of day E3.5 ICM derived NOD ES cells in the presence of KP, KP/CH or PD/CH and derivation of stable ES lines. Images of initial colonies observed at passage 2 after embryo dissociation are shown together with representative images at later passages. (D) Karyotype of NOD ES lines. (E) Immunostaining of NOD ES lines for pluripotency markers. Inhibitors used during cell line derivation and propagation are indicated. (F) Chimerism in adult mice generated from the indicated cell lines is evident by the presence of agouti (brown) and albino coat color originating from the NOD background. Lower panel demonstrates germline transmission obtained from a male NOD ES #43 derived chimera that was mated to an NOD female. (G) Real-Time PCR analysis for endogenous expressed Oct4, c-Myc and Klf4 genes in NOD iPS and ES cells grown in the indicated conditions. Relative expression levels are normalized to levels detected in control 129 ES cells. (H) Southern analysis indicating correct targeting of the endogenous Nanog locus in NOD ES cells with Nanog-GFP knock-in targeting construct.

FIGS. 5(A)-5(H) illustrate generation of NOD EpiSC-like ES and iPS cells. (A) Dox dependent NOD-iPS lines were trypsinized and plated on MEF feeders and grown in mESM without Dox. Representative image of colonies typically observed 5-8 days after plating that can be manually passaged using collagenase and stably propagated in epiESM conditions. (B) Southern blot analysis indicating identical integration pattern for Sox2 transgene in NOD-iPS #1 and its derived cell line NOD EpiSC-like iPS #1. Black triangle indicate endogenous band, white triangle indicates transgenic band. (C) Derivation of Epiblast-like ES cells from NOD blastocysts by plating the embryos in EpiSC derivation medium and passaging ICM outgrowths after 5-8 days in epiESM conditions. Continued culture in epiESM and manual dissociation supported the growth of colonies with flat morphology-like colonies. (D) Immunofluorescent staining for pluripotency markers in EpiSC, EpiSC-like NOD iPS and ES lines. (E) Teratoma formation of NOD Epiblast-like NOD ES and iPS cell lines. (F) Evaluation of Oct4 distal enhancer (DE) and proximal enhancer (PE) reporter gene activity in the indicated pluripotent lines. Baseline activity was analyzed by infecting with an empty vector. (G) Cell samples were equally divided and plated on feeders in the indicated growth medium, and 24 hours later the wells were supplemented with either JAKi or ALKi or were kept without any inhibitor. At Day 6 the wells were stained for Oct4 to determine the relative percentage of pluripotent colonies. Values indicated are normalized to internal control sample (Growth medium without inhibitors—Gray Bars) in which each cell line were not exposed to inhibitors and defined as 100%. Relative percentage lower than 30% was defined as “sensitivity” to the presence of the inhibitor. (H) Whole genome cluster analysis of transcripts from pluripotent cell lines analyzed. Top 5000 differentially expressed probes were selected by cross-array standard deviations of the normalized expression values and included in unbiased cluster analysis. Growth conditions and passage number for the cell lines are indicated.

FIGS. 6(A)-6(F) show the resetting of the identity of pluripotent states. (A) 129 EpiSC cells derived from the epiblast of day E5.5 mouse embryos were reprogrammed into ICM-like cells after infection with TetO-Klf4 or c-Myc lentiviruses and culture in the mESM+Dox. Images on the bottom show representative colony formation observed 4-6 days after infection, which were passaged on MEF feeders. The cell lines were termed Epi-iPS. (B-C) Evaluation of Oct4 enhancer activity and sensitivity to JAKi and ALKi on the converted cell lines was performed as indicated in (FIG. 4 E-F). (D) Chimeric mice generated from the indicated Epi-iPS cells lines as evident by the agouti coat color. (E) Efficiencies of iPS derivation from EpiSC and somatic cells: 129 EpiSCs were infected with Teto-Klf-4-2A-mOrange or Teto-cMyc-2A-mOrange lentiviruses and after 3 days of Dox inductions mOrange+ infected cells were single cell sorted in 96 well plates. Different hematopoietic cells from transgenic reprogrammable mice carrying the Dox inducible OSKM transgenes were single cell sorted in 96 well plates. HSC—hematopoietic stem cell enriched fraction, CMP—common myeloid progenitors. Efficiency for Nanog+ cells stable in mESM was determined at day 25 for all samples. Average results from 2 independent experiments are shown. *P value<0.05 for the outlined efficiency comparisons. (F) Converting NOD EpiSC-like ES cells into transgene dependent ICM-like cells. EpiSC like cells were generated from NOD-ES line #43 after withdrawal of KP/CH and growing the cells in epiESM. As in (A) the line was infected with TetO-K and within 2 passages in mESM in the presence of Dox. Stable lines termed Epi-iPS were derived. The lower pictures indicate that the cell line required Dox or the presence of KP/CH to remain stable in culture. Factors and growth conditions utilized to facilitate the identity conversion at each step are indicated. The pluripotency state was evaluated and defined based on Oct4 enhancer activity and ALKi or JAKi sensitivity (indicated in blue or red).

FIG. 7 illustrates that pluripotent states are “metastable.” Depicted is a model summarizing the requirements for in vitro stabilization of different pluripotent states in 129 (left half) and NOD mouse strains (right half). “Metastability” pertains to describing a system with two or more equilibrium states (indicated by the stippled grey horizontal lines) that can interconvert by defined signals. The ICM-like state is characterized by a greater developmental potential than the EpiSCs-like state. The factors required for stabilizing the respective pluripotent states in the different genetic backgrounds are indicated in green on the plateau lines for each state. The ICM-like pluripotent state in permissive 129 cells, whether achieved by direct in vitro reprogramming or by ICM explantation is stabilized by Lif/Stat3 signaling, while the bFGF/Activin/Nodal signaling stabilizes the EpiSC-like state. Defined transcription factor or small molecules convert the EpiSC-like cells to the ICM-like ES cell state (highlighted in orange on the left). The 129 and NOD EpiSC-states are indistinguishable in their stability and growth condition requirements and can be reverted into ICM-like pluripotency by expression of Klf4 or c-Myc or by specific inhibitors added to the medium. However, the ICM-ES like state is unstable on the NOD genetic background and requires continuous expression of exogenous factors in addition to Lif (dashed black and red arrows).

FIGS. 8(A)-8(G) show the derivation of “naïve” mouse ESC-like induced pluripotent stem cells. (A) Strategy used for reprogramming C1 human fibroblasts transgenic for DOX-inducible lentiviruses (TetO) encoding OCT4, SOX2, KLF4. Representative images of C1 cultures and subcloned cell line #C1.2 observed at different stages during reprogramming (P indicates passage number). NOD mESCs and C1.2 hiPSC cells after DOX withdrawal are also shown. (B) C1 hiPSC line maintained in conventional bFGF/serum supplemented human ES growth conditions (hESM) was transferred into N2B27 PD/CH/LIF+DOX, and emerging colonies where subcloned. Representative C1.10 hiPSC clone is shown. (C) Representative immuno-staining results for pluripotency markers. (AP-alkaline phosphatase). (D) Karyotype analysis at the indicated passages. (E) Sections of differentiating teratomas after injection of naïve C1.10 hiPSC. (F) Signaling dependence of pluripotent cell lines. Pluripotent cells were equally divided and plated on feeders in the indicated growth medium in which these cell lines are normally maintained, and 36 hours later the wells were supplemented with the indicated inhibitors or growth factors. After 6 days, wells were fixed and stained for Nanog to determine the relative percentage of pluripotent colonies. Colony formation is normalized to an internal control “growth medium only” without inhibitors. Normalized percentages lower than 25% are defined as “sensitivity” to the presence of the supplemented inhibitor. (G) C1.2 hiPSC line was electroporated with mammalian expression vectors expressing the indicated reprogramming factors and cells were subjected to puromycin selection and passaged in PD/CH/LIF without of DOX. Values indicate relative percentage of SSEA4+ colonies obtained in comparison to control cells that were transfected with an Oct4/Klf4/Sox2 encoding polycistronic construct.

FIGS. 9(A)-9(D) show the in vitro stabilization of naïve human pluripotent stem cells. (A) Screening of factors that allow propagation of transgene-independent (i.e., DOX-independent) C1-hiPSCs in PD/CH/LIF supplemented media and effect of the removal of individual factors from the pool of 13 small molecules or cytokines on the stabilization of pluripotent C1 hiPSCs independent of DOX. C1 cells were plated on feeders in N2B27 media with the indicated factors. p-values using student's t-test indicates significant change in comparison to cells grown in DOX/PD/CH/LIF conditions, which were defined as a control (100% survival). (B) Scheme for reverting hESCs to generate “naive-hESCs”. (C) Representative images of WIBR3 hESCs at different stages of the reversion process in PD/CH/LIF/FK (P indicates passage number). (D) Representative images of naive-WIBR3.5 hESC line, derived by plating WIBR3 hESCs in PD/CH/FK/LIF medium without transfection of exogenous factors. Magnifications of captured images are indicated.

FIGS. 10(A)-10(G) illustrate the molecular and functional characteristics of naive-hESC lines. (A) Karyotype of human pluripotent stem lines at the indicated passages. (B) Single-cell cloning efficiency of different pluripotent stem cell lines as determined by the number of wells containing Nanog+colonies after 7 days. (C) Cell doubling time of various mouse and human pluripotent stem cell lines. Plated cells were plated in triplicates and counted at 1, 4 and 7 days after plating and increase in cell number was used to extrapolate average doubling time. Error bars represent s.d., and p-values using student's t-test indicates significant difference in the average of hESC/hiPSC lines in comparison to the average of naive-hESC/hiPSC lines. (D) Bisulphite sequencing methylation analysis of the endogenous human Oct4 and Nanog promoters. (E) Immunostaining for pluripotency markers of representative naive-hESC line maintained in PD/CH/FK/LIF conditions. (F) Representative images of differentiation of naïve-hESCs by EB formation. Multi-lineage differentiation was determined by quantitative RT-PCR and compared to undifferentiated hESCs (normalization control). Gene expression was normalized to the average expression in BGO1 hESCs. (G) Representative hematoxylin and eosin stainings of teratoma sections. (H) Directed in vitro differentiation of naïve-C1.2 hiPSCs into Pax6+/Nestin+ neural precursors and neuron-specific class III β-tubulin+ (TUJ1+)/Map2+ differentiated neurons.

FIGS. 11(A)-11(F) show that naive-hESCs share defining signaling and epigenetic features with mESCs, (A) Signaling dependence of pluripotent cell lines. Pluripotent cells were equally divided and plated on feeders in the indicated growth medium in which these cell lines are normally maintained, and 36 hours later the wells were supplemented with the indicated inhibitors or growth factors. After seven days, wells were fixed and stained to determine the relative percentage of colonies positive for pluripotency markers. SSEA1 staining was used for mouse stem cells. Colony formation was normalized to an internal control “Growth medium” without inhibitors (first left column). Normalized percentages lower than 5% are defined as “sensitivity” to the presence of the supplemented inhibitor. (B-C) Western blot analysis of total ERK1/2, phosphorylated ERK1/2 (pERK1/2) and phosphorylated Stat3 (pStat3). WIBR3 hESC and C1 hiPSC were grown in traditional bFGF/Activin containing media while naive-hESC/hiPSCs were grown in N2B27 PD/CH/FOR/LIF. ‘2×LIF’ indicates two fold increase in LIF concentration. (D) LIF/Stat3 is required for stabilization of naive-hESC/hiPSCs. Naïve NOD mESCs, WIBR3.5 and WIBR3.1 naive-hESC, and C1 naïve-hiPSCs were electroporated with a pBRY-CAGGS-flox-DsRedT4-IP control plasmid, a plasmid encoding a dominant negative Stat3 Y705F mutant (Stat3-DN), or a plasmid Stat3-C constitutively active mutant (pBRY-Stat3-CA). Cells were passaged three times in the presence of puromycin. After 20 days, colonies positive for pluripotency markers were counted and normalized to colonies from cells electroporated with empty vector (n=3 replicates for each condition). (E) RT-PCR expression of early germ cell markers in the presence or absence of BMP4/7/8 cytokines for 7 days. (F) Representative FISH analysis for XIST RNA (red) and Cot1 nuclear RNA (green). Pri-WIBR3.2 cell line was analyzed after passaging in conventional bFGF/serum-containing human ESC growth conditions. Numbers indicate average percentage of XIST positive nuclei counted.

FIGS. 12(A)-12(E) show that naive-hESC/hiPSCs share a global transcriptional profile with mESCs. (A) Hierarchical clustering of the top 500 genes differentially expressed genes on different hESCs lines and naive-hESCs/hiPSCs using Pearson correlation and average linkage. Heatmap indicates expression values, which are row-centered and row-normalized. Numbers on trees indicate Pearson correlation coefficients, and tree branch heights are proportional to distances between samples. (B) Bar chart showing median expression ratio of pluripotency and lineage-specific marker genes in hESCs and naïve-hESCs. Asterisks delineate genes in which the false discovery rate (FDR) was <0.1 between the naïve and primed group of samples. (C) Quantitative RT-PCR for gene expression levels of the indicated genes in comparison to GAPDH. Expression is relative to WIBR3-hESCs. p-value using student's t-test was used in comparing between hESC/hiPSC and naïve-hiPSC/hESC sample groups. (D) Histogram of surface expression of human and mouse MHC class I alleles using FACS analysis on the indicated cell lines. Black graph indicates isotype match control. (E) Cross-species gene expression clustering where mESCs and naïve-hESCs formed a distinct group apart from mEpiSCs and hESCs. The legend is shown on right with yellow and blue indicating positive and negative correlation, respectively. Gene expression relative abundance (RA) was clustered by Spearman correlation and average linkage. pvclust was used to assess confidence of the clustering tree as detailed in Methods section. The AU and BP p-values were shown on the left and right of the branches, respectively. Mouse samples are labeled in purple and human samples are labeled in brown.

FIG. 13 demonstrates metastable states of pluripotency. Depicted is a model describing relationships between genetic background and requirements for exogenous factors to achieve stabilization of the naïve (ICM-like or ESC-like) and primed (epiblast-like or EpiSC-like) pluripotent states. “Metastability” pertains to describing a system with two or more in vitro stable states that can interconvert by defined signals. The naive state is characterized by a greater developmental potential and more immature epigenetic and functional characteristics in comparison to the primed state. The transcription factors and culture supplements minimally required for interconversion and stabilization of the respective pluripotent states in the different genetic backgrounds are highlighted.

FIGS. 14(A)-14(B) illustrate staining properties of cell lines. (A) Immuno-straining for pluripotency markers in NOD iPS lines growing in the absence of Dox and in the presence of the indicated compounds or conditioned medium. (B) NOD iPS cell-derived teratomas stained by Hematoxyline and Eosin.

FIGS. 15(A)-15(B) illustrate replacement of Klf4 during iPS generation by PD/CH small molecules. (A) Previously described secondary transgenic MEF line (Markoulaki et al., Nature Biotechnology 2009) carrying the Oct4, Sox2 and c-Myc dox inducible lentiviral transgenes (abbreviated as OSM) were grown in the presence of Dox supplemented mESM with or without PD and CH inhibitors. Initial colonies were observed at day 16 which started to acquire ES-like morphology with passagin. Doxycycline and inhibitors were withdrawn from the culture media at day 30 and iPS line were stably maintained afterwards and continued to express pluripotency makers by immuno-staining (B). IPS lines could not be derived in the absence of Dox or each of the two inhibitors used. These defined conditions failed to replace any of the other reprogramming factors (Oct4, Sox2 and c-Myc) (data not shown). One of 2 independent experiments is shown.

FIGS. 16(A)-16(B) show derivation of RAT iPS cells. (A) Tail tip fibroblasts were derived from adult rats, and infected with Dox inducible lentiviruses encoding Oct 4, Sox 2, Klf4, c-Myc and a constitutive lentivirus encoding rtTA as indicated in FIG. 1a. Initial colonies were observed 10-16 days after infection and Dox induction, and subcloned cell lines were grown in mESM supplemented with KP/CH and generated Dox independent iPS lines that expressed pluripotency markers, and generated differentiated teratomas after injection in immuno-compromised mice (B).

FIG. 17 shows ERK phosphorylation in ES cells under different growth conditions. Immunoblot analyses of steady state levels of phosphor (Thr202, Tye204)-ERK (pERK) in V6.5 ES cells treated for 48 hours in KP/CH. PD/CH were used as a positive control as PD has been characterized as an inhibitor of erk phosphorylation. These results indicate that KP/CH at the concentrations used in the study does not induce inhibition in Erk phosphorylation. MEF cells transgenic for Dox inducible c-Myc and Klf4 reprogramming factor were used. Dox induction of the factors for 48 hours failed to demonstrate any change in Erk phosphorylation in fibroblasts. Western blotting for beta-actin (loading control) is shown. Results from one out of two independent experiments are shown.

FIG. 18 illustrates the cell cycle analyses on NOD derived ES cells. Cell cycle analysis as determined by FACS detection of 7-AAD staining. NOD ES lines derived and grown in the presence of specific combinations of small molecules displayed identical cell cycle profile to that of wild type V6.5 mES cells.

FIG. 19 shows the karyotype of NOD EpiSC-like ES line #2.

FIGS. 20(A)-20(B) demonstrate that 129 ES and iPS cells propagated in epiESM conditions are stable and fully pluripotent. (A) Tail tip fibroblasts were infected with Ubi-rtTA and Dox inducible lentiviruses encoding Oct4, Sox2, c-Myc and Klf4 Cell were grown in epiESM+Dox for 30 days, and IPS colonies were further passaged independent of Dox in epiESM. Cells were passaged by trypsinization and were stable in culture. iPS-TTF #3 line shown in the fugre generated adult chimeras with germline transmission as evident by derivation of agouti pups after mating the chimeras with BDF1 females, (B) ES lines were derived from day 3.5 blastocysts and grown in epiESM condition as indicated in the experimental procedures section. Lines were stably propagated by trypsinization and were capable of generating of adult chimeras with germline transmission (Table S1).

FIG. 21 illustrates the dependence of pluripotency states on distinct signaling pathways. 129 mES or EpiSC cell samples were equally divided and plated on irradiated MEFs in the indicated growth conditions (mESM or epiESM) in which these cell lines are normally maintained, and 24 hours later the wells were supplemented with either JAKi or ALKi or were kept without any inhibitor. Representative images at Day 6 of the experiment are shown. EpiSC cells as previously described, differentiated upon introduction of ALK inhibitors, while mES cells differentiate upon blocking of Stat3 signaling by the JAK inhibitor.

FIGS. 22(A)-22(B) show characterization of pluripotency markers. (A-B) Semi-quantitative RT-PCR analysis of transcripts from pluripotency genes and actin in the indicated pluripotent lines.

FIGS. 23(A)-23(B) show characterization of iPS^{EpiSC} cells. (A) Immuno-fluorescence strain of 129 Epi-iPS cells for Oct4, Nanog and SSEA1. (B) Southern blot analysis for detection of viral integration for lentiviruses encoding the reprogramming factors. Black arrows indicate endogenous bands, and empty arrows indicate integration bands. Klf4 was probed with an internal digest (EcoRI) which detects ^{˜}1.2 kb transgene band and with an external XbaI digest. *Indicate background bands preset in all samples.

FIGS. 24(A)-24(B) show small molecule conversion of distinct pluripotent states. (A) 120 EpiSC cells derived from the epiblast of day E5.5 mouse embryos or NOD EpiSC-like ES cells and were reprogrammed into ICM-like after culture in the mESM+KP/CH. While most colonies differentiated due to withdrawal of bFGF and change of medium (indicated by asterisks), ES-like colonies could be observed after 7-9 days and passaged via trypsinization. Within 2 passages ICM-like ES lines could be stably generated. (B) However, NOD EpiSC-like #43 line which was derived from NOD-ES #43 by withdrawing KP/CH and growing the cells in epiESM conditions for over 8 passages (FIG. 6F), remained dependent on KP/CH molecules. Factors and growth conditions utilized to facilitate the identity conversion at each step are indicated in parenthesis. The pluripotency state was evaluated and defined based on Oct4 enhancer activity, inhibitor sensitivity (indicated in red or blue accordingly for each line). Identical results were obtained by using PD/CH inhibitor combination or KP alone (data not shown).

FIGS. 25(A)-25(C) show generation of transgene-dependent hiPSCs with mESC growth and functional properties. C1 human fibroblasts transgenic for DOX-inducible lentiviruses (TetO) encoding OCT4, SOX2 and KLF4. (A) Representative images of C1 fibroblast cultures and subcloned cell lines “C1.1” in Kenpaullone (KP) and CH supplemented media. Different culture conditions are indicated in the panels (P indicates passage number). C57/B6 mESCs in the PD/CH culture conditions are shown for comparison. (B) Normal karyotype of C1 hiPSC lines is observed at different passages. (C) Immunostaining for pluripotency markers of a representative hiPSC clone maintained on DOX. AP: Alkaline Phosphatase. D. Teratomas derived from DOX-dependent C1 hiPSC lines. Note that after injection the cells where not exposed to DOX in vivo. E. DNA fingerprinting analysis on C1.1 hiPSC clone demonstrating genetically identity to the primary C1 hiPSC line previously reported in Hockemeyer et al. Cell Stem Cell, 2008 and ruling out contamination from other pluripotent cells.

FIGS. 26(A)-26(B) show work defining exogenous factors required to sustain naïve pluripotency in vitro for human cells. (A) C1.1 and C1.2 hiPSC lines required DOX and small molecules PD/CH/LIF or KP/CH/LIF for propagation. Withdrawal of any of these exogenous factors resulted in their differentiation as demonstrated after 2 passages in the images. (B) C1.4 hiPSC grown in N2B27 PD/CH/LIF+Dox media in the absence of feeders, on fibronectin/laminin coated plates. Normal karyotype, SSEA4 staining and a derived teratoma are shown.

FIGS. 27(A)-27(D) show pre-X inactivation epigenetic state in naïve human pluripotent stem cells. (A) C1.7.1 hiPSC subclone was obtained following electroporation of a parental cell line C1.7 hiPSC with puromycin resistance gene encoding construct and selecting the cells with puromycin. Subsequently the line was continuously passaged in PD/CH/LIF+DOX conditions or in traditional bFGF containing hES media (hESM), termed clone C1.7.1. FISH analysis for XIST coating is shown on both cell lines, and on embryonic bodies (EBs) generated from the C1.7.2. (B) Southern blot analysis for puromycin resistance allele show identical transgene digest pattern in naïve-C1.7.1 hiPSCline and subclone C1.7.2 grown in bFGF containing media. White triangle indicates bands from DR4 mouse feeder cells harboring transgene, while the black triangle represents band from the exogenous gene in hiPSC. (C) Teratoma formation from C1.7.1 hiPSC and C1.7.2 hiPSC clones demonstrating that both clones are pluripotent in vitro despite the difference in growth conditions and X chromosome inactivation status. This analysis provides conclusive genetic proof for a clonal relation between the cell lines, and that individual cells can assume distinct states of pluripotency in vitro depending on the exogenous factors provided. (D) Bisulfite sequencing analysis of six CpG sites in single clones of an XIST promoter amplicon represented previously described (Lengner et al. Cell. 2010).

FIGS. 28(A)-28(G) show naïve hESCs transgenic for Oct4 and Klf4 propagated in PD/CH/LIF. (A) WIBR3 and BGO1 hESCs were stably transfected with a vector encoding Oct-4-2A-Klf4 polycistronic insert and subjected to puromycin drug selection in PD/CH/LIF. (B) Representative images after selection from the indicated lines. P indicates passage number. (C) Bisulphite methylation analysis of endogenous Oct4 and Nanog promoters. (D) Differentiated teratomas generated in vivo from the indicated lines. P indicates passage number. (E) BGO1-OK.3 cells differentiated upon withdrawal of LIF, PD, or CH. dsRED+ cells were sorted after transfection with pTurbo-cre plasmid to isolate cells that looped out Oct4-2A-Klf4 transgene and plated in PD/CH/LIF conditions. Pluripotent cells could not be maintained in the absence of Oct4 and Klf4 transgenes. (F) DNA finger printing genetic analysis on BGO1-OK.5 line. (G) Southern blot analysis on SpeI digested genomic DNA for dsRED marker cassette to detect transgenes in transduced hESCs. Genomic integrations are highlighted with red arrows.

FIG. 29 illustrates the transcriptional changes induced by Forskolin treatment. Quantitative RT-PCR for Klf2, Klf4, Oct4, and Sox2 gene upon treatment of hESCs or mouse EpiSCs with 10 mM of forskolin (FK) for 48 hours is shown. Relative expression is shown in comparison to untreated controls (defined as a level of 1). p-value using student's t-test indicates significance change (*p<0.01) in gene expression levels.

FIG. 30 illustrates the efficiency of converting primed pluripotent cells into naïve pluripotent cells in different species. Efficiencies of converting 129 and NOD mEpiSCs and hESCs into naïve TGFβ/Activin independent pluripotent state in different conditions. EpiSCs were treated with ROCKi and plated as single cells on MEFs for 36 hours, and then surviving cells were grown in N2B27 media supplemented with the indicated small molecules and growth factors for 20 days, and TGF inhibitor was added for the last 6 days to verify independence of activin/nodal signaling. Clonal populations with Oct4+/SSEA4+ and TGFβ resistant pluripotent cells were quantified. Average results from 2 experimental replicates are shown. Note that generation of naïve-hESCs without transfection with Klf2/4 is extremely low and thus is nearly below detection threshold for this assay. All comparisons between strains and species yielded statistically different behavior [* indicates p<0.0001, two-way analysis of variation (ANOVA)]. This analysis highlights the influence of the genetic background on the requirements for exogenous factor supplementation and efficiencies for stabilizing naïve pluripotency in vitro.

FIGS. 31(A)-31(B) show characterization of naïve-WIBR3.5 hESC line. (A) Immunostaining for pluripotency markers of Phosphatase. (B) Teratomas derived from naïve-WIBR3.5 hESC. AP: Alkaline demonstrating differentiated cells from three germ layers. P indicates passage number at which testing was conducted.

FIG. 32 shows Stat3 signaling stabilizes human naïve pluripotent stem cells. Naive-WiBR3.5 stably transfected with constitutive active Stat3 mutant (pBRY-Stat3-CA) remains pluripotent in PD/CH/FK and the absence of LIF, as evident by staining for pluripotency markers and in vivo teratoma formation.

FIG. 33 demonstrates evaluation of in vitro differentiation of human pluripotent cells into PGC precursors. Stable transfection of a Vasa-EGFP reporter (reported in Kee et al. Nature, 2010) into C1 hiPSCs and G418 selection yielded a “C1-VasaRep” subclone. This subclone was then epigenetically converted to a naïve state to generate the naive C1-VasaRep hiPS cell line. Only the parental C1-VasaRep hiPS cells demonstrated significant GFP induction specifically in the presence of BMP4/7/8 inducing factors at days 5 and 7. The genetically identical naïve clone did not show induction of VASA GFP+ cell formation upon exposure to BMP4/7/8 after 7 days, consistent with lack of enhanced induction of Blimp1, VASA and DAZL expression.

FIG. 34 shows Oct4 enhancer activity in human pluripotent stem cells. Evaluation of human Oct4 distal enhancer (DE) and proximal enhancer (PE) reporter gene activity in the indicated pluripotent lines is shown. Baseline activity was analyzed by infecting with an empty vector. p-value using student's t-test was used in comparing Distal enhancer activity between hiPSC/hESCs and naive-hiPSC/hESCs.

### DETAILED DESCRIPTION OF CERTAIN EMBODIMENTS OF THE INVENTION

The present invention relates to methods and compositions for modifying the pluripotency state of a mammalian cell. In some embodiments the invention relates to methods for deriving or culturing vertebrate ES cells or iPS cells. In some embodiments, the invention provides methods and compositions useful for stabilizing a cell in a pluripotent state. The invention provides methods of deriving ES cells from species (e.g., humans) or strains that have not heretofore been amenable to derivation of ES cells (e.g., naïve ES cells). In some embodiments the invention provides methods of deriving ES cells from mice of a strain that is less permissive for deriving ES cells than the 129 strain. In some embodiments the invention provides methods of deriving ES cells from mice of a strain considered in the art to be “non-permissive” for deriving ES cells. In some embodiments the invention provides methods of deriving ES cells from mice of a strain for which there is no reproducible, art-accepted method of deriving germline-competent ES cells or in which one or more standard techniques of deriving mouse ES cells typically or essentially always fail to yield germline-competent ES cells. In some embodiments ES cells cultured according to the invention exhibit one or more improved properties relative to ES cells of the same species cultured using standard techniques. In some aspects, the invention provides isolated pluripotent cells, e.g., ES cells, ES cell lines, ES clones, and cell cultures containing them.

A “pluripotent” cell has the ability to self-renew and to differentiate into cells of all three embryonic germ layers (endoderm, mesoderm and ectoderm) and, typically, has the potential to divide in vitro for a long period of time, e.g., at least 20, at least 25, or at least 30 passages, or more (e.g., up to 80 passages, or up to 1 year, or more), without losing its self-renewal and differentiation properties. A pluripotent cell is said to exhibit or be in a “pluripotent state”. A pluripotent cell line or cell culture is often characterized in that the cells can differentiate into a wide variety of cell types in vitro and in vivo. Cells that are able to form teratomas containing cells having characteristics of endoderm, mesoderm, and ectoderm when injected into SCID mice are considered pluripotent. Cells that possess ability to participate in formation of chimeras (upon injection into a blastocyst of the same species that is transferred to a suitable foster mother of the same species) that survive to term are pluripotent. If the germ line of the chimeric animal contains cells derived from the introduced cell, the cell is considered germline-competent in addition to being pluripotent.

ES cells are pluripotent cells. ES cells have been derived from mice, primates (including humans), and some other species. ES cells are often derived from cells obtained from the inner cell mass (ICM) of a vertebrate blastocyst but can also be derived from single blastomeres (e.g., removed from a morula). Pluripotent cells can also be obtained using somatic cell nuclear transfer in at least some species, e.g., mice and various non-human primates. Pluripotent cells can also be obtained using parthenogenesis, e.g., from germ cells, e.g., oocytes. Other pluripotent cells include embryonic carcinoma (EC) and embryonic germ (EG) cells. See, e.g., Yu J, Thomson J A, Pluripotent stem cell lines. 22(15):1987-97, 2008.

Genetic background has been found to affect efficiency of ES cell derivation in mice. Mouse ES cells have been most commonly derived from the 129 or C57BL/6 strains. Prior to the present invention efforts to derive ES cells from non-obese diabetic (NOD) mice, a strain widely used to study insulin-dependent diabetes mellitus have failed.

Induced pluripotent stem (iPS) cells are pluripotent, ES-like cells derived from somatic cells (e.g., fibroblasts, keratinocytes, hematopoietic cells, neural precursor cells) by a process termed “reprogramming”. As used herein, the term “reprogramming protocol” refers to a set of manipulations (e.g., introduction of vector(s) carrying particular genes) and/or culture conditions (e.g., culture in medium containing particular compounds) that generates pluripotent cells from somatic cells. Reprogramming was first achieved by infecting cells with retroviruses that encode the transcription factors Oct4, Sox2, Klf4, and c-Myc (termed “OSKM factors”) under control of a viral LTR. Oct4, Sox2 and Klf4 (“OSK factors”) are also sufficient to reprogram mouse or human somatic cells to pluripotency, although with lower efficiency than if c-Myc is included. Other sets of reprogramming factors, e.g., Oct4, Sox2, Nanog, and Lin28 (OSNL factors) can be used to reprogram human cells, with Lin28 being dispensable. The ectopically expressed factors induce expression of endogenous pluripotency genes such as Oct4 and Nanog. Since the retroviral vectors in iPS cells derived by this approach are silenced, maintenance of pluripotency relies on expression of such endogenous genes and establishment of an appropriate transcriptional network in the reprogrammed cells. iPS cells can be generated using a variety of different reprogramming protocols. For example, iPS cells can be generated using multiple vectors or using a single polycistronic vector (Carey B W, et al., Reprogramming of murine and human somatic cells using a single polycistronic vector. 106(1):157-62, 2009; Sommer, C A, et al., iPS Cell Generation Using a Single Lentiviral Stem Cell Cassette. Stem Cells. 2008 Dec. 18. [Epub ahead of print]); They can be generated by infecting somatic cells with retroviruses (e.g., lentiviruses) carrying the reprogramming factors as defined doxycycline (Dox)-inducible transgenes (Wernig M, et al., A drug-inducible transgenic system for direct reprogramming of multiple somatic cell types. Nat. Biotechnol., 26(8):916-24, 2008; Hockemeyer D, A drug-inducible system for direct reprogramming of human somatic cells to pluripotency. Cell Stem Cell; 3(3):346-53, 2008). Cells infected with Dox-inducible transgenes encoding the factors reprogram upon Dox exposure. Dox can be withdrawn after about 20-30 days without apparently affecting the pluripotent state. So-called “secondary” iPS cells can be generated from cells obtained from mice that carry defined combinations of drug-inducible reprogramming factors (Markoulaki S, Transgenic mice with defined combinations of drug-inducible reprogramming factors. Nat. Biotechnol., 27(2):169-71, 2009). iPS cells have been generated in which the introduced genomic copies of the reprogramming factors have been removed by Cre-mediated recombination (Soldner F, et al., Parkinson's disease patient-derived induced pluripotent stem cells free of viral reprogramming factors. Cell., 136(5):964-77, 2009) or using non-integrating vectors (Stadtfeld M, et al., Induced pluripotent stem cells generated without viral integration, Science, 322(5903):945-9, 2008; Yu, J. et al., Human Induced Pluripotent Stem Cells Free of Vector and Transgene Sequences. Science. Mar. 26, 2009 [epub ahead of print] or using transient transfection (Okita, K., et al., Science, 322(5903):949-53, 2008).

Compounds have been identified that mimic the effect of certain of the reprogramming factors in protocols for generating iPS cells. Such compounds, referred to herein as “reprogramming compounds” can be used instead of the corresponding reprogramming factors in protocols for deriving iPS cells. For example, Wnt pathway stimulation (e.g., using Wnt3a conditioned medium) was shown to promote reprogramming in cells infected with OSK viruses, thereby replacing use of c-Myc virus in such protocols. See, e.g., PCT/US2008/010249 (WO/2009/032194). Mikkelson, T S, et al., Dissecting direct reprogramming through integrative genomic analysis, Nature, 454(7200):49-55, 2008; Shi, Y., et al., Cell Stem Cell 2, 525-528, 2008; Shi, Y., et al., Cell Stem Cell, 3:568-574, 2008. The histone deacetylase inhibitor valproic acid was reported to reprogram human fibroblasts without use of Klf4 and c-Myc (Huangfu, D., et al., Induction of pluripotent stem cells from primary human fibroblasts with only Oct4 and Sox2. Nat. Biotechnol. 26(11):1269-75, 2008)

As used herein, a compound is said to “replace” a reprogramming factor in generating iPS cells if (i) somatic cells cultured in medium containing the compound become reprogrammed with a detectable efficiency when infected with retroviruses encoding a set of reprogramming factors that would otherwise not be sufficient to cause detectable reprogramming; or (ii) somatic cells cultured in medium containing the compound and infected with retroviruses encoding a set of reprogramming factors lacking the factor become reprogrammed with an efficiency at least 5-fold, at least 10-fold, at least 25-fold, at least 50-fold, or at least 100-fold as great as would be the case had the medium not contained the compound. For example, a compound “replaces c-Myc in generating iPS cells” if somatic cells cultured in medium containing the compound and infected with retroviruses encoding only the OSK factors (or treated with Dox in the case of cells that contain the factors as Dox-inducible transgenes) become reprogrammed with an efficiency at least 5-fold, at least 10-fold, at least 25-fold, at least 50-fold, or at least 100-fold as great as would be the case had the medium not contained the compound. A compound “replaces Klf4 in generating iPS cells” if (i) somatic cells cultured in medium containing the compound become reprogrammed with a detectable efficiency when infected with retroviruses encoding only the OSM factors; or (ii) somatic cells cultured in medium containing the compound and infected with retroviruses encoding only the OSM factors become reprogrammed at a level at least 5-fold, at least 10-fold, at least 25-fold, at least 50-fold, or at least 100-fold as great as would be the case had the medium not contained the compound. A compound “replaces both Klf4 and c-Myc” if (i) somatic cells cultured in medium containing the compound become reprogrammed with a detectable efficiency when infected with retroviruses encoding only the OS factors; or (ii) somatic cells cultured in medium containing the compound and infected with retroviruses encoding only the OS factors become reprogrammed at a level at least 5-fold, at least 10-fold, at least 25-fold, at least 50-fold, or at least 100-fold as great as would be the case had the medium not contained the compound. It is understood that “replace a reprogramming factor” refers to replacing ectopic expression of the factor, e.g., expression from a gene that was introduced into a cell (or ancestor thereof) such as by a retroviral vector, non-integrating vector, or transient transfection, and does not imply replacement of expression of the factor from an endogenous gene. “Efficiency” refers to the percentage of somatic cells subjected to a reprogramming protocol that become reprogrammed, e.g., that form colonies composed of reprogrammed cells. Reprogramming efficiency can be measured as known in the art, e.g., by assessing expression of a suitable reporter, e.g., a fluorescent protein such as GFP operably linked to an Oct4 or Nanog promoter. It is understood that the determination whether a compound replaces a factor is typically made using culture conditions that are suitable for deriving iPS cells using the standard techniques and cell types described above. It is also understood that the timing of reprogramming achieved using a compound that replaces a factor may differ from that achieved using the factor. For example, it may take longer to derive an iPS cell using a compound than would be the case using the factor that it replaces.

The invention encompasses the recognition that pluripotent cells have the potential to adopt at least two alternate pluripotency states: an “ICM-like state” having properties characteristic of ES cell derived from the ICM of 129 mice, and an “EpiSC-like state” having properties characteristic of ES cell derived from the epiblast of 129 mice. The invention is based in part on the discovery that certain cells, e.g., cells derived from the ICM of certain mouse strains or human pluripotent cells, exhibit an unstable pluripotent state, e.g., when subjected to conditions that result in derivation of stable ES or iPS cells when applied to other strains such as 129. In the absence of appropriate stimuli, these cells rapidly differentiate or convert to an EpiSC-like state. Like EpiSC, they exhibit a restricted ability to participate in formation of chimeras or contribute to the germline. The invention is also based in part on the unexpected discovery of particular conditions which result in the stabilization of the pluripotent state of resistant (i.e., less permissive or non-permissive) cells (e.g., mouse cells, human cells).

For example, the invention relates in part to the discovery that persistent, e.g., constitutive, expression of Klf4 and/or c-Myc, or culture in the presence of compounds that replace Klf4 or c-Myc expression in generating induced pluripotent stem (iPS) cells, stabilizes a pluripotent state in cells in which a stable pluripotent state would not result using “standard techniques” for deriving ES or iPS cells. Attempts to derive iPS cells from NOD mouse embryonic fibroblasts (MEFs) using Dox-inducible lentiviruses resulted in cells that, while possessing the key characteristics of ES cells, differentiated upon Dox withdrawal. Surprisingly, it was found that constitutive expression of Klf4 or c-Myc, but not Oct4 or Sox2, was sufficient to enable the derivation of stable NOD iPS cells. It was also found that constitutive expression of Klf4 or c-Myc was also sufficient to allow derivation of stable ES cells from NOD ICM cells, whereas such cells failed to yield any ES cells under conditions routinely used for ES cell derivation. In addition, culturing NOD ICM or epiblast-derived cells in medium containing compounds that replace c-Myc or Klf4 in generating iPS cells had similar effects in enabling the derivation of stable NOD ES and iPS cell lines as did constitutive expression of c-Myc or Klf4. Upon withdrawing the compounds, the cells convert to an EpiSC-like state. In addition, EpiSC cells derived from the 129 strain can be converted to an ES-like state in which they participate in chimera formation and contribute to the germline. These results demonstrate that expression of Klf4 or c-Myc, or culture in medium containing compounds that replace Klf4 or c-Myc in generating iPS cells, promotes conversion to, and stabilization of, an ICM-like state. Without wishing to be bound by theory, the inventors suggest that ICM cells of certain strains and species adopt an EpiSC-like rather than ICM-like state when subjected to standard techniques intended to derive ES cells, thereby hampering derivation of ES cells that generate chimeras and contribute to the germline. In addition, adoption of an EpiSC-like rather than ICM-like state may be responsible for various properties of certain ES cells, e.g., human ES cells, such as failure to propagate after dissociation (thus hampering single cell cloning) and lack of amenability to genetic modification. In some aspects, the invention provides methods for improving the properties of such ES cells.

The invention also relates to the discovery that conventional human ESCs can be reprogrammed into a more immature/less restricted state that extensively shares defining features with pluripotent mouse ESCs. This was achieved by ectopic induction of Oct4, Klf4 and Klf2 factors combined with LIF and inhibitors of glycogen synthase kinase 3β (GSK313) and mitogen-activated protein kinase (ERK1/2) pathway. Forskolin, a protein kinase A pathway agonist that can induce Klf4 and Klf2 expression, transiently substitutes for the requirement for ectopic transgene expression. In contrast to conventional human ESCs, these epigenetically converted cells have growth properties, X chromosome activation state (XaXa), a gene expression profile, and signaling pathway dependence that are highly similar to those of mouse ESCs. Finally, the same growth conditions allow the derivation of human induced pluripotent stem (iPS) cells with similar properties as mouse iPS cells.

“Standard techniques” for deriving mouse ES cells refer to techniques that routinely yield germline-competent ES cells when applied to cells obtained from the ICM of blastocysts of the 129 mouse strain. An exemplary standard technique is one in which embryonic cells are isolated from the blastocyst ICM, and ICM outgrowths are cultured in mESM conditions prior to their transfer to a pseudopregnant female mouse, e.g., as described in the Markoulaki, et al., “Somatic cell nuclear transfer and derivation of embryonic stem cells in the mouse”, Methods, 45(2):101-14, 2008 and/or Meissner A, et al., Derivation and manipulation of murine embryonic stem cells, Methods Mol. Biol., 482:3-19, 2009. See also, Nagy, A., et al., Manipulating the Mouse Embryo, 3^{rd} ed., Cold Spring Harbor Laboratory Press, Cold Spring Harbor, N.Y. “mESM conditions” refer to culturing cells on irradiated mouse embryonic fibroblasts (MEFs) in DME containing 15% fetal calf serum (FCS), leukemia inhibiting factor (LIF), 1 mM glutamine, 1% nonessential amino acids, 0.1 mM mercaptoethanol. Other techniques as described herein may include those in which compounds (e.g., leukemia inhibitory factor (LIF), bone morphogenetic protein (BMP), or the MEK inhibitor PD098059) are used to replace the MEF feeder layer and/or serum as known in the art. “Standard techniques” for deriving human ES cells typically involve use of a MEF or human cell feeder layer and serum or, if cultured in serum-free medium, compounds such as bFGF. For example, the ICM of a human blastocyst is removed by immunosurgery, dissociated in Ca2+-Mg2+-free medium, and plated over mouse embryonic fibroblasts or human feeder cells (Thomson et al., Science 282, 1145 (1998). The mouse cells are irradiated to suppress their proliferation. See, e.g., B. E. Reubinoff et al., Nature Biotechnol. 18, 399, 2000; Mitalipova M & Palmarini G. Isolation and characterization of human embryonic stem cells. Methods Mol. Biol. 331:55-76, 2006; Ilic D, et al., Derivation of hESC from intact blastocysts. Curr Protoc Stem Cell Biol., Chapter 1:Unit 1A.2, 2007; Ludwig T, A Thomson J., Defined, feeder-independent medium for human embryonic stem cell culture. Curr Protoc Stem Cell Biol. Chapter 1:Unit 1C.2, 2007. It will be understood that culture conditions can be feeder layer free. It will also be understood that the culture conditions can include the use of matrices such as laminin, Matrigel™, and the like. In some embodiments, methods recently described in Chen A E, et al., Optimal timing of inner cell mass isolation increases the efficiency of human embryonic stem cell derivation and allows generation of sibling cell lines. Cell Stem Cell. 4(2): 103-6, 2009, are used.

A “cell culture medium” (also referred to herein as a “culture medium” or “medium”) is a medium for culturing cells containing nutrients that maintain cell viability and support proliferation. The cell culture medium may contain any of the following in appropriate amounts and combination: salt(s), buffer(s), amino acids, glucose or other sugar(s), antibiotics, serum or serum replacement, and other components such as peptide growth factors, etc. Cell culture media ordinarily used for particular cell types are known to those skilled in the art. For example, cell culture media of use for deriving and maintaining pluripotent cells are known in the art. As used herein, “standard medium” refers to cell culture medium used in standard techniques for deriving or culturing ES cells. In some embodiments standard medium is chemically defined medium. In some embodiments standard medium is serum-free medium, e.g., mTeSR1™ medium (StemCell Technologies, Vancouver, BC). In some embodiments standard medium comprises a serum replacement composition.

The invention provides a method of deriving a pluripotent vertebrate cell, the method comprising (a) providing a vertebrate cell suitable for deriving a pluripotent cell; (b) causing the cell to persistently express Klf4 or c-Myc or culturing the cell in medium that contains a compound that replaces Klf4 or c-Myc expression in generating iPS cells; and (c) maintaining the cell in culture under conditions suitable to produce a pluripotent vertebrate cell. The cell may be cultured for a time sufficient to produce an ES cell or ES cell colony. An ES cell colony may contain, e.g., at least 50, at least 100, or at least 200 ES cells. The time period employed may be about the same as used in standard techniques for deriving ES cells or iPS cells. In some embodiments the time required to result in a colony of a desired size or cell number is reduced when the inventive methods are used as compared with the time required when standard techniques are used. In some embodiments ES cell derivation is considered to have been achieved when one or more cell colonies having morphology indicative of ES cells are evident. In some embodiments ES cell derivation is considered to have been achieved when one or more cell colonies expressing markers or reporters indicative of ES cell identity are evident.

The invention provides a method of deriving a vertebrate ES cell, the method comprising: (a) providing a vertebrate cell isolated from the ICM of a vertebrate blastocyst or isolated from the epiblast of a vertebrate embryo; (b) causing the cell to persistently express Klf4 or c-Myc or culturing the cell in medium that contains a compound that replaces Klf4 or c-Myc expression in generating iPS cells; and (c) maintaining the cell in culture under conditions suitable to produce a vertebrate ES cell. In some embodiments, expression of Klf4 or c-Myc is constitutive. “Constitutive” expression refers to expression that persists over time under many or most culture conditions, e.g., it is not silenced and is typically under control of a promoter that is active in many or most culture conditions and does not require presence of particular inducing conditions. Constitutive promoters active in mammalian cells are known in the art. In one embodiment a constitutive promoter is the ubiquitin promoter. Persistent expression refers to constitutive expression or to regulatable expression under conditions that result in expression. For example, persistent expression includes inducible expression under inducing conditions (e.g, the inducer is present) or repressible expression in the absence of repressing conditions (e.g., the repressor is absent). In some embodiments, “persistent expression” refers to expression for at least 4 weeks, e.g., at least 5, 6, 7, 8, 10, 15, or 20 weeks, or longer. In some embodiments, “persistent expression” refers to expression that continues beyond the time at which retroviral vectors used to induce reprogramming would have become substantially silenced (e.g., expression has diminished by at least a factor of 10, 50, or 100-fold). In some embodiments, persistent expression is at a level at least 25%, or at least 50%, of that typically achieved in reprogramming protocols in which c-Myc or Klf4 retrovirus or Dox-inducible expression is used, or at about the same level as in such protocols. Often, causing a cell to express Klf4 or c-Myc entails introducing a gene encoding the protein into a cell (or an ancestor of the cell) or treating a cell into which such gene has been introduced so as to induce expression.

It will be understood that the reprogramming factors used in generating iPS cells and/or used in the methods of derivation and culture of the present invention are often from the same species as the cell in which such factors are to be ectopically expressed. For example, in deriving an iPS cell from a mouse somatic cell, the murine factors are often used, whereas in deriving an iPS cell from a human somatic cell, the human factors are often used. It will also be understood that minor differences in sequence of the factors may occur among different strains or individuals of a particular species and also that minor changes in sequence for example, an isolated replacement of a leucine with an isoleucine or valine, an aspartate with a glutamate, a threonine with a serine, or a similar replacement of an amino acid with a structurally related amino acid and/or one whose side chain has similar biochemical properties (e.g., conservative substitutions) will typically not have a major effect on the biological activity of the resulting molecule. Conservative amino acid substitutions may be made on the basis of similarity in polarity, charge, solubility hydrophobicity, hydrophilicity, and/or the amphipathic nature of the residues involved. For example, non-polar (hydrophobic) amino acids include alanine, leucine, isoleucine, valine, proline, tryptophan, and methionine; polar/neutral amino acids include glycine, serine, threonine, cysteine, tyrosine, asparagine, and glutarmine; positively charged (basic) amino acids include arginine, lysine, and histidine; and negatively charged (acidic) amino acids include aspartic acid and glutamic acid. The invention contemplates that reprogramming factors having sequences identical to, or, e.g., at least 90%, 95%, 96%, 97%, 98%, 99% or more identical to the sequences of the factors used herein or otherwise used in the art for reprogramming over, e.g., at least 90%, 95%, 96%, 97%, 98%, 99% or 100% of the sequence, allowing the introduction of gaps, may be used. One may use commonly available methods and software programs to calculate percent identity.

In some embodiments culturing a cell in medium that contains a compound that replaces Klf4 or c-Myc in generating iPS cells comprises culturing the cell for at least 5 days, e.g., between 5-10 days, 1-20 days, 20-30 days, or longer. In some embodiments medium is replaced with fresh medium, e.g., about every 48 hours, to sustain activity of the compounds, or the medium is supplemented with fresh compound. In some embodiments the cell is used in a method of deriving a non-human mammal or in a method that involves differentiating the cell, e.g., into a desired cell type, within 24 hours of having been withdrawn from culture in medium containing the compound or within 24 hours of having been withdrawn from conditions that maintain constitutive Klf4 or c-Myc expression. In some embodiments, concentrations of the compounds used are as described in the Exemplification, or within a factor of 0.5 to 10-fold the concentrations described in the Exemplification. In some embodiments the concentration is between 0.75 and 2-fold, or between 0.75 and 5-fold, the concentration(s) exemplified herein. It will be understood that the concentrations can be optimized for a given cell type and that lower or higher concentrations may be used.

In some embodiments, the invention provides methods for deriving ES cells from species or strains that have been heretofore considered “non-permissive” for generating ES cells. In some embodiments, “non-permissive” refers to a strain or species that does not detectably yield ICM-like pluripotent cells using standard techniques. In some embodiments “non-permissive” refers to a strain or species that yields ES cells with an efficiency less than 1%, in some embodiments less than 0.1% of that with which the 129 strain yields ES cells using standard techniques. In some embodiments, the invention provides methods for deriving ES cells from species or strains that have been heretofore considered non-permissive for generating ES cells that participate in chimera formation. In some embodiments, the invention provides methods for deriving ES cells from species or strains that have been heretofore considered non-permissive for generating ES cells that can contribute to the germline (“germline-competent” ES cells).

The inventive methods may be applied to derive or culture pluripotent cells of any strain, e.g., mouse strain, or substrain of interest. Numerous strains and substrains are available from The Jackson Laboratory (Bar Harbor, Me.) (http://www.jax.org), e.g., those strains and substrains listed in the JAX® Mice database, which is incorporated herein by reference, or from Taconic (Hudson, N.Y.) or other commercial suppliers. Pluripotent cells, e.g., ES cells or iPS cells, derived or cultured according to the present invention can be derived from cells obtained from inbred or non-inbred strains or species. In some embodiments a pluripotent ES or iPS cell is derived from an inbred strain, e.g., of mice, while in other embodiments the cell is derived from a non-inbred strain. In some embodiments an inbred strain has surpassed 20 generations of inbreeding. In some embodiments non-human animals, e.g., mice, of an inbred strain are homozygous at virtually all of their loci. In some embodiments an inbred strain has a unique set of characteristics that sets it apart from all other inbred strains. In some embodiments, a species or strain, e.g., a mouse strain, is useful for research on apoptosis, autoimmune disease, cancer, cardiovascular disease, cell biology, dermatology, development, diabetes and/or obesity, endocrine deficiency, hearing (or hearing loss), hematological research, immunology, inflammation, musculoskeletal disorders, neurobiology, neurodenerative disease, metabolism, vision (or vision loss), reproductive biology, or infectious disease. In some embodiments a mouse is a laboratory mouse. In some embodiments a mouse is of the species Mus musculus or a subspecies thereof. In some embodiments a mouse is a hybrid of different subspecies, e.g., Mus musculus domesticus and Mus musculus musculus. Exemplary mouse strains of interest are, e.g., the 16 JAX® Mice strains whose genome has been sequenced: strain C57BL/6J, 129S1/SvImJ, A/J, AKR/J, BALB/cByJ, BTBR T+tf/J, C3H/HeJ, CAST/EiJ, DBA/2J, FVB/NJ, MOLF/EiJ, KK/HIJ, NOD/ShiLtJ, NZW/LacJ, PWD/PhJ, and WSB/EiJ. Other mouse strains of interest are CD-1, CBA, ICR, and Balb/C. In some embodiments the pluriopotent cell is derived from a strain that has a mutation (which may be an identified or defined mutation or may be an unknown mutation) or phenotype of interest, e.g., susceptibility to a condition, e.g., a condition that affects other species, e.g., humans, or may exhibit features of a condition that affects other species, e.g., humans. In some embodiments a mouse strain has a phenotype that mimics a human disease.

In some embodiments the mouse is of a strain that has been considered in the art to be “non-permissive” for derivation of ES cells. The non-obese diabetic (NOD) mouse is an exemplary strain that has been heretofore considered non-permissive for ES cell derivation. NOD mice develop spontaneous autoimmune diabetes, which shares many similarities to autoimmune or type 1a diabetes in humans (see, e.g., Makino S, et al., “Breeding of a non-obese, diabetic strain of mice”. Jikken Dobutsu 29 (1): 1-13, 1980; Kikutani H, Makino S., “The murine autoimmune diabetes model: NOD and related strains”. Adv. Immunol. 51: 285-322, 2002; Anderson M S, Bluestone J A. The NOD mouse: a model of immune dysregulation. Annu Rev Immunol., 23:447-85, 2005). The invention provides methods of deriving a NOD ES cell, e.g., a germline-competent NOD ES cell. In some embodiments the NOD ES cell is derived from a cell obtained from the ICM of a NOD blastocyst or from the epiblast of a NOD embryo. The invention also provides NOD ES cells, e.g., germline-competent NOD ES cells. In one embodiment, a method of deriving a NOD ES cell comprises (a) providing a cell isolated from the ICM of a blastocyst of a NOD mouse or isolated from the epiblast of a NOD embryo; (b) causing the cell to persistently express Klf4 or c-Myc or culturing the cell in medium that contains a compound that replaces Klf4 or c-Myc expression in generating iPS cells; and (c) maintaining the cell in culture under conditions suitable to produce a NOD ES cell. The cell may be cultured for a time sufficient to produce an ES cell or ES cell colony. An ES cell colony may contain, e.g., at least 20, at least 30, at least 50, at least 100, or at least 200 ES cells. A NOD strain can be, e.g., NOD/ShiLtJ (formerly NOD/LtJ). It is contemplated to derive ES or iPS cells from any NOD mice or related strains, e.g., NOD mice that have genetic modifications. It is also contemplated to derive ES or iPS cells from progeny or descendants resulting from crossing NOD mice with mice of other strains. In some embodiments a NOD mouse is a an immunocompromised mouse, e.g., a NOD-SCID mouse. In some embodiments, the inventive methods or compositions are used to derive or culture a mouse ES cell having an H-2^{g7} MHC haplotype.

In some embodiments the methods have an efficiency of at least 1%, e.g., at least 5%, in generating ES cells, e.g., NOD ES cells. In some embodiments the methods have an efficiency of between 1% and 20%, or between 5% and 20%, in generating ES cells, e.g., NOD ES cells. In some embodiments the methods have an efficiency of greater than 20% in generating ES cells, e.g., up to 30%, 40%, 50%, 60%, or more. In some embodiments the compositions and methods of the invention are applied to improve the efficiency with which ES cells can be derived, e.g., from strains in which currently available methods yield ES cells with poor efficiency (e.g., Balb/C). For example, the efficiency may increase by a factor of at least 1.5, e.g., between 1.5 and 10-fold, or even more. In some embodiments the efficiency increases from below 1% to 1% or greater, or to 5% or greater. In some embodiments the efficiency increases from below 5% to 5% or greater, or to 10% or greater. In some embodiments the efficiency increases from below 10% to 10% or greater, e.g., to 20% or greater.

The compositions and methods of the invention may be applied to derive or culture pluripotent cells from rodent species, e.g., rats, rabbits, hamsters, guinea pigs, etc. The invention may be applied to derive or culture pluripotent cells from primates, e.g., non-human primates, or humans. In many embodiments, the vertebrate is a mammal. In some embodiments the mammal is a bovine, ovine, caprine, equine, canine, or feline. It is also envisioned that compositions and methods of the invention may be used to derive pluripotent cells, e.g., ES cells or iPS cells from non-mammalian vertebrates, e.g., zebrafish or other non-mammalian organisms of interest such as birds. In some embodiments, if the species is one from which ES or iPS cells have not heretofore been derived, techniques and culture conditions can be adapted from standard techniques used in other species, e.g., related species.

Compositions and methods of the invention can be applied in the derivation or culture of pluripotent cells derived from cells obtained from any of a variety of sources. For example, cells obtained from the inner cell mass (ICM) or epiblast can be used to derive ES cells. In some embodiments, the compositions and methods are applied to derive pluripotent stem cells from blastomeres, e.g., blastomeres isolated from a morula or from a 4-8 cell stage embryo. In some embodiments, the compositions and methods are applied to derive pluripotent stem cells from germ cells. In some embodiments the compositions and methods are used to derive pluripotent cells using parthenogenesis or SCNT. In some embodiments the methods are applied to derive or culture induced pluripotent stem (iPS) cells.

The invention provides cell culture medium comprising (i) medium suitable for deriving an ES cell from an ICM cell obtained from a blastocyst of a 129 mouse or for culturing such ES cell; and (ii) a compound that replaces Klf4 or c-Myc in generating iPS cells. The invention provides cell culture medium comprising (i) medium suitable for deriving a human ES cell from an ICM cell obtained from a human blastocyst or suitable for culturing such human ES cell; and (ii) a compound that replaces Klf4 or c-Myc in generating iPS cells. In some embodiments, the medium suitable for deriving an ES cell from an ICM cell obtained from a blastocyst of a 129 mouse does not already contain significant amounts of compound that replaces Klf4 or c-Myc. The medium suitable for deriving or culturing a mouse ES cell can be any medium used in standard techniques for deriving or culturing mouse ES cells. The medium suitable for deriving or culturing a human ES cell can be any medium used in standard techniques for deriving or culturing human ES cells. In some embodiments, the medium suitable for deriving or culturing a mouse ES cell does not already contain significant amounts of compound that replaces Klf4 or c-Myc. In some embodiments, the medium suitable for deriving or culturing a human ES cell does not already contain significant amounts of compound that replaces Klf4 or c-Myc. By “significant amounts” are meant amounts at least 25% of the amounts used in the art to replace Klf4 or c-Myc in generating iPS cells. In some embodiments, the inventive medium contains at least 2, at least 5, at least 10 times as much compound that replaces Klf4 or c-Myc as present in standard media. In some embodiments, the inventive medium contains between 0.5 and 10 times as much compound that replaces Klf4 or c-Myc as used in reprogramming protocols to generate iPS cells. In some embodiments the inventive medium contains about the same amount, or between 1 and 5 times as much compound as used in reprogramming protocols to generate iPS cells.

The invention provides compositions comprising a pluripotent vertebrate ES cell, e.g., a pluripotent mouse or human ES cell and a compound that replaces Klf4 or c-Myc in generating iPS cells. The composition often further comprises cell culture medium, e.g., standard medium for deriving or culturing ES cells.

In some embodiments the invention provides improvements in methods or compositions for deriving or culturing ES cells, wherein the improvement comprises providing a compound that replaces Klf4 or c-Myc in generating iPS cells in a cell culture medium used for deriving or culturing ES cells. In some embodiments the improvement comprises deriving or culturing ES cells using medium that contains a small molecule that replaces Klf4 or c-Myc in generating iPS cells. In some embodiments the improvement is an improvement to any technique known in the art for deriving or culturing ES cells. In some embodiments the invention provides improvements in methods or compositions for deriving or culturing iPS cells wherein the improvement comprises providing an increased amount of a compound that replaces Klf4 or c-Myc in a cell culture medium used for deriving or culturing iPS cells or wherein the improvement comprises culturing iPS cells in medium that comprises a compound that replaces Klf4 or c-Myc, e.g., for at least 5, 6, 8, 10, 12, or 15 weeks, or indefinitely. In some embodiments the improvement comprises culturing an iPS cell under conditions in which Klf4 or c-Myc is persistently, e.g., constitutively, expressed. In some embodiments an ES cell or iPS cell is derived using standard techniques and, after derivation, is cultured in medium containing a compound that replaces Klf4 or c-Myc. Such culture may continue for, e.g., at least 2, 3, 5, 7, or 10 days, or at least 2, 3, 4, 5, 6 weeks, or more. In some embodiments an ES cell or iPS cell is derived without using a compound that replaces Klf4 or c-Myc and, after derivation, is cultured in medium containing a compound that replaces Klf-4 or c-Myc. Such culture may continue for, e.g., at least 2, 3, 5, 7, or 10 days, or at least 2, 3, 4, 5, 6 weeks, or more.

A variety of compounds that replace Klf4 or c-Myc in generating iPS cells can be used in the invention. In some embodiments a compound that replaces c-Myc is a Wnt pathway activator. In some embodiments a compound that replaces Klf4 is a GSK3 inhibitor. In some embodiments a compound that replaces Klf4 or c-Myc is a small molecule, by which is meant an organic compound having multiple carbon-carbon bonds and a molecular weight of less than 2,000 daltons. In some embodiments a compound replaces Klf4 but does not replace c-Myc. In some embodiments a compound replaces c-Myc but does not replace Klf4. In some embodiments at least two compounds are used, one of which replaces Klf4 and one of which replaces c-Myc.

In some embodiments of the invention a compound that replaces Klf4 in generating iPS cells is a paullone. Paullones were initially identified as inhibitors of cyclin dependent kinases (CDKs) (Schultz, C., et al., J. Med. Chem., 42, 2909-2929, 1999; Zaharevitz, D., et al., Cancer Res, 59, 2566-2569, 1999; Leost, M., et al., Eur. J. Biochem., 267, 5983-5994, 2000). See also U.S. Patent Pub. No, 20030181439. An exemplary paullone scaffold is shown below:

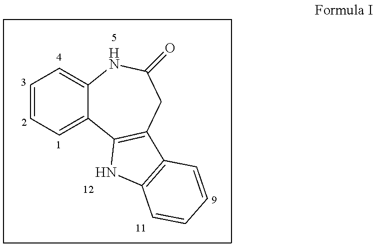

In some embodiments a paullone of use in the invention is substituted at the 2-, 3-, 4-, 9-, and/or 11-position(s). In some embodiments the paullone is substituted at the 9 position. In some embodiments the paullone is substituted at the 9 position and at one additional position. In some embodiments a substituent is or comprises a halogen (e.g., bromine, chlorine). In some embodiments the paullone is mono-substituted. In other embodiments the paullone has two or more substituents. In some embodiments a substituent is or comprises an alkyl (e.g., lower alkyl, such as C₁₋₆ alkyl (e.g., methyl, ethyl), or amino-alkyl chain. In some embodiments the paullone is kenpaullone (9-bromo-7,12-dihydroindolo[3,2-d][1]benzazepin-6(5H)-one).

In some embodiments a compound that replaces c-Myc is a Wnt pathway activator. Wnts are a family of secreted proteins important for various developmental and physiological processes (Mikels, A J and Nusse, R., Oncogene, 25: 7461-7468, 2006). Wnts interact with members of the Frizzled (Fz) and low-density-lipoprotein receptor-related protein (LRP) families to stimulate intracellular signal transduction cascades known as the canonical pathway (reviewed by Logan C Y and Nusse, R Annu. Rev Cell Dev. Biol., 20:781-810, 2004) and non-canonical pathways (reviewed by Kohn, A D and Moon, R T, Cell Calcium, 38: 439-446, 2005). Briefly, Wnt signaling via the canonical pathway leads to diminished phosphorylation of β-catenin, thereby promoting its stabilization and nuclear localization. As used herein, a “Wnt pathway activator” is a compound that, when contacted with cells with an intact Wnt signaling pathway (e.g., expressing receptors for Wnt and expressing other molecules that function in the Wnt signaling pathway), causes increased intracellular levels of β-catenin, increased nuclear translocation of β-catenin, and/or changes in gene expression characteristic of cells contacted with a biologically active Wnt protein. A Wnt pathway activator may physically interact with a Wnt receptor or may act indirectly by physically interacting with one or more intracellular components of the Wnt signaling pathway such as a kinase or phosphatase that acts on β-catenin.

In some embodiments of the invention a Wnt pathway activator is a biologically active Wnt protein (a “Wnt”). A cell of interest may be contacted with biologically active Wnt protein by culturing the cell in Wnt-conditioned medium. “Wnt-conditioned medium” refers to medium in which cells that secrete biologically active Wnt protein have been cultured, such that biologically active Wnt is present in the medium. Biologically active Wnt protein may, e.g., be isolated from naturally occurring sources (e.g., mammalian cells that naturally produce the protein) or produced in cells using recombinant expression technology. See, e.g., U.S. Pat. Pub. No. 20040248803 and Willert, K., et al., Nature, 423: 448-52, 2003. In certain embodiments the Wnt pathway activator comprises soluble, biologically active Wnt3a, which in some embodiments is provided by Wnt3a conditioned medium. In some embodiments the Wnt pathway activator is a compound that is that replaces soluble, biologically active Wnt3a protein in generating iPS cells.

In some embodiments a compound that replaces c-Myc or Klf4 is a GSK3 inhibitor. GSK3 is a serine/threonine kinase (reviewed in Frame and Cohen, Biochem J 359:1-16, 2001). In some embodiments, a “GSK3 inhibitor” is a compound that inhibits the kinase activity of GSK3β. The compound may or may not also inhibit GSK3a and may or may not inhibit one or more additional kinases. Many potent and selective small molecule inhibitors of GSK3 are known (see, e.g., Wagman A S, Johnson K W, Bussiere D E, Curr Pharm Des., 10(10):1105-37, 2004). In some embodiments the GSK3 inhibitor has an IC50 for GSK3β of 0.5 μm or less. Exemplary GSK3 inhibitors are: (1) BIO: (2′Z,3′E)-6-Bromoindirubin-3′-oxime (Polychronopoulos, P. et al. J. Med. Chem. 47, 935-946, 2004). (2) AR-A014418: N-(4-Methoxybenzyl)-N′-(5-nitro-1,3-thiazol-2-yl)urea. (Bhat, R., et al., J. Biol. Chem. 278, 45937-45945, 2003). (3) SB 216763: 3-(2,4-Dichlorophenyl)-4-(1-methyl-1H-indol-3-yl)-1H-pyrrole-2,5-dione. See, e.g., Smith, D. G., et al, Bioorg. Med. Chem. Lett. 11, 635-639, (2001) and Cross, D. A., et al., J. Neurochem. 77, 94-102, (2001), (4) SB 415286: 3-[(3-Chloro-4-hydroxyphenyl)amino]-4-(2-nitrophenyl)-1H-pyrrol-2,5-dione. (Smith, D. G., et al, Bioorg. Med. Chem. Lett. 11, 635-639, 2001; Coughlan, M. P., et al, Chem. Biol. 10, 793-803, 2000, (5) TDZD-8: 4-Benzyl-2-methyl-1,2,4-thiadiazolidine-3,5-dione; (6) CHIR-911 and CHIR-837 (also referred to as CT-99021 and CT-98023, or CHIR-99021 and CHIR-98023, respectively). Chiron Corporation (Emeryville, Calif.) and related compounds; (7) lithium chloride; (8) sodium valproate; and (9) GSK3 inhibitor II (Calbiochem). Additional GSK3 inhibitors are described in U.S. Pat. Nos. 6,057,117 and 6,608,063; U.S. patent application publications 20040092535, 20040209878, 20050054663; WO/2003/049739; WO/2002/085909, WO/2003/011287, WO/2005/039485, and WO/2006/091737.

In some embodiments of interest the GSK3 inhibitor is CHIR-99021 or a structurally related compound (Cline, G W, et al., Diabetes 51:2903-2910, 2002). A structurally related compound may have the same core structure but have one or more substituents, e.g., a lower alkyl group (e.g., methyl, ethyl), may have one or more different functional groups, etc.

In some embodiments a paullone, e.g., kenpaullone, is used in combination with a GSK3 inhibitor, e.g., CHIR-99021 or a structurally related compound.

In some embodiments a compound that inhibits the ERK cascade is used in addition to a compound that inhibits Klf4 or c-Myc. An exemplary ERK cascade inhibitor is PD184352. Structurally related or unrelated ERK cascade inhibitors could be used.

It is contemplated to use compounds, e.g., small molecules, that enhance reprogramming, such as histone modifiers (e.g., HDAC inhibitors) or DNA methyltransferase inhibitors, in combination with a compound such as those described above that replaces Klf4 or c-Myc in generating iPS cells. In some embodiments of the invention a compound that replaces Klf4 or c-Myc is not a histone deacetylase (HDAC) inhibitor. In some embodiments a compound that replaces Klf4 or c-Myc in generating iPS cells has at least one biological activity in somatic cells (e.g., fibroblasts) other than (e.g., in addition to or instead of) activity as an HDAC inhibitor.

The invention provides pluripotent cells, cell lines, and cell clones derived or cultured using the inventive methods and/or compositions. The invention further provides cell cultures, wherein at least some of the cells in the cell culture are derived or cultured using the inventive methods and/or compositions. “Cell line” refers to a population of largely or substantially identical cells, wherein the cells have often been derived from a single ancestor cell or from a defined and/or substantially identical population of ancestor cells. For example, a cell line may consist of descendants of a single cell. A cell line may have been or may be capable of being maintained in culture for an extended period (e.g., months, years, for an unlimited period of time). It will be appreciated that cells may acquire mutations and possibly epigenetic changes over time such that some individual cells of a cell line may differ with respect to each other. In some embodiments at least 80%, 85%, 86%, 87%, 88%, 89%, 90%, 91%, 92%, 93%, 94%, 95%, 96%, 97%, 98%, or 99% of the cells of a cell line or cell culture are at least 95%, 96%, 97%, 98%, or 99% genetically identical. In some embodiments at least 80%, 85%, 86%, 87%, 88%, 89%, 90%, 91%, 92%, 93%, 94%, 95%, 96%, 97%, 98%, or 99% of the cells of a cell line or cell culture express the same set of cell surface markers. The set of markers could be markers indicative of pluripotency or cell-type specific markers. A cell “clone” refers to a population of cells derived from a single cell. It will be understood that if cells of a clone are subjected to different culture conditions or if some of the cells are subjected to genetic modification, the resulting cells may be considered distinct clones. As used herein, the term “cell culture” refers to a composition comprising a plurality of viable cells wherein at least some of the cells are proliferating, e.g., not cell cycle arrested. A cell culture could be composed of cells from one or more different cell lines or sources.

In certain embodiments of the invention, a pluripotent cell line or cell clone of the invention is stable in culture. As used herein, a state, condition, or property is “stable” if it remains substantially unchanged over a time period of interest, e.g., exhibits little or no variability over such time period. “Stabilize” refers to promoting the establishment and/or maintenance of a stable state, condition, or property, e.g., by inhibiting or preventing a change in such state, condition, or property. A cell or cell line or cell clone is stable in culture if it continues to proliferate over multiple passages in culture (e.g., indefinitely), most or all cells in the culture (e.g., at least 90%, 95%, 97%, 98%, or more) are of the same type or differentiation state (e.g., are pluripotent), and cells resulting from cell division are of the same cell type or differentiation state. Thus a stabilized cell or cell line retains its “identity” in culture as long as the culture conditions are not altered, and the cells continue to be passaged appropriately. In some embodiments, methods and compositions of the invention enhance or promote existence of a stable pluripotent state. In some embodiments the pluripotent state is an ICM-like state, rather than an EpiSC-like state. Thus in some embodiments the invention is a method for converting a pluripotent cell from an EpiSc-like state to an ICM-like state or for stabilizing a pluripotent cell in an ICM-like state. In some embodiments, the pluripotent state is characterized by cell colonies that morphologically resemble those of ES cells of the 129 strain. In some embodiments, the pluripotent state, e.g., in mice, is characterized by ability to participate in chimera formation with frequencies at least 20% of that of ES cells of the 129 strain. In some embodiments, the pluripotent state, e.g., in mice, is characterized by ability to contribute to the germ line in chimeras with frequencies at least 20% of that of ES cells of the 129 strain. In some embodiments the pluripotent state is characterized by colonies that morphologically resemble those of ES cells of the 129 strain. In some embodiments the pluipotent state is characterized by maintenance of both X chromosomes (in XX lines) in an activated state. In some embodiments a pluripotent state has at least 2, 3, 4, or more of the foregoing properties. In some embodiments an inventive cell line or clone has a stable pluripotency state. In some embodiments an inventive cell line or clone is karyotypically stable.

One of skill in the art will be aware of ways to assess the stability of a cell population. One suitable method is to examine the expression of “markers” known in the art to be characteristic of cells of a particular type or differentiation state. For example, stage-specific embryonic antigens-1, -3, and -4 (SSEA-1, SSEA-3, SSEA-4) are glycoproteins specifically expressed in early embryonic development and are markers for ES cells (Solter and Knowles, 1978, Proc. Natl. Acad. Sci. USA 75:5565-5569; Kannagi et al., 1983, EMBO J. 2:2355-2361), with SSEA-1 being a marker of mouse ES cells and SSEA-3 and -4 being markers of human ES cells. Elevated expression of the enzyme alkaline phosphatase (AP) is another marker associated with undifferentiated embryonic stem cells (Wobus et al., 1984, Exp. Cell 152:212-219; Pease et al., 1990, Dev. Biol. 141:322-352). Additional ES cell markers are described in Ginis, I., et al., Dev. Biol., 269: 369-380, 2004 and in Adewumi 0, et al., Nat. Biotechnol., 25(7):803-16, 2007 and references therein. For example, TRA-1-60, TRA-1-81, GCTM2 and GCT343, and the protein antigens CD9, Thy1 (also known as CD90), NANOG, TDGF1, DNMT3B, GABRB3 and GDF3, REX-1, TERT, UTF-1, TRF-1, TRF-2, connexin43, connexin45, Foxd3, FGFR-4, ABCG-2, and Glut-1 are of use. In an exemplary embodiment a mouse pluripotent stem cell line, e.g., a mouse ES cell line, e.g., a NOD-ES cell line, expresses Oct4, Nanog, and SSEA-1. In an exemplary embodiment a human pluripotent stem cell line, e.g., a human ES cell line, expresses Tra 1-60, Nanog, Oct4, Sox2, and SSEA3 and/or SSEA4.

In some embodiments, at least 80%, at least 90% of the pluripotent stem cells of a colony, cell line, or cell culture express one or more marker(s), e.g., a set of markers, indicative of pluripotency. In some embodiments at least 91%, 92%, 93%, 94%, 95%, 96%, 97%, 98%, 99%, or more of the cells of a colony, cell line, or cell culture express the marker(s).

Gene expression profiling may be used to assess pluripotency state. Pluripotent cells, such as embryonic stem cells, and multipotent cells, such as adult stem cells, are known to have a distinct pattern of global gene expression. See, for example, Ramalho-Santos et al., Science 298: 597-600, 2002; Ivanova et al., Science 298: 601-604, 2002; Boyer, L A, et al. Nature 441, 349, 2006, and Bernstein, B E, et al., Cell 125 (2), 315, 2006. One may assess DNA methylation, gene expression, and/or epigenetic state of cellular DNA, and/or developmental potential of the cells, e.g., as described in Wernig, M., et al., Nature, 448:318-24, 2007. Other methods of assessing pluripotency state include epigenetic analysis, e.g., analysis of DNA methylation state.

In certain embodiments of the invention, a pluripotent stem cell line, e.g., an ES or iPS cell line, derived or cultured according to the invention, e.g., a human ES or iPS cell line, a non-human vertebrate ES or iPS cell line, a mouse ES or iPS cell line, e.g., NOD ES line or NOD iPS line, has a normal karyotype. In certain embodiments at least 50%, 55%, 60%, 65%, 70%, 75%, 80%, 85%, 90%, or greater than 95% of cells in metaphase examined exhibit a normal karyotype. In certain embodiments, at least 50%, 55%, 60%, 65%, 70%, 75%, 80%, 85%, 90%, or greater than 95% of cells exhibit a normal karyotype after at least 5, 6, 7, 8, 9, 10, 11, 12, 13, 14, 15, 20, or more passages, e.g., 25 passages, or 30 passages, or more. In some embodiments normal karyotype comprises having the correct number of chromosomes without evidence of translocation or deletion or duplication. In some embodiments normal karyotype comprises having a normal banding pattern. In some embodiments a karyotype is normal karyotype based on analysis by fluorescence in situ hybridization (FISH). In some embodiments a pluripotent stem cell or cell line is an XO cell or cell line which, in some embodiments is otherwise karyotypically normal.

The invention contemplates that properties of existing pluripotent cell lines, e.g., existing human ES cell lines, or cell lines developed in the future, may be modified by culturing them according to the present invention, e.g., in culture medium that contains a compound that replaces Klf4 or c-Myc in generating iPS cells. In some embodiments, pluripotent cells, e.g., ES cells, derived or cultured according to the invention exhibit reduced sensitivity to dissociation, e.g., they exhibit improved survival after dissociation relative to ES cells derived or cultured using standard techniques. In some embodiments, ES cells derived or cultured according to the invention exhibit increased amenability to single cell cloning, e.g., they exhibit increased number of clones derived from single cells when cultured following dissociation, relative to ES cells derived or cultured using standard techniques. A cell population is considered “dissociated” if at least 50%, 60%, 70%, 80%, 90%, 95%, 96%, 97%, 98%, 99% or more of the cells are singlets or doublets. Cells may be dissociated using various techniques, e.g., trypsinization or treatment with other enzymes that disrupt cell-cell contacts and/or adherence to substrates, mechanically, e.g., using pipetting or rubber policeman, or combinations of techniques. In some embodiments survival or number of clones resulting when ES cells are plated after dissociation is increased by at least a factor of 2, e.g., between 2- and 5-fold, or between 5- and 10-fold, or between 10- and 50-fold, or more, e.g., up to 100-fold, 500-fold, 1,000-fold, or even higher using methods of the invention relative to standard techniques. In some embodiments at least 10%, at least 25%, at least 50%, at least 75%, or at least 90% of such clones are composed of pluripotent cells. In some embodiments human ES cells derived or cultured using the inventive methods exhibit a cloning efficiency after dissociation of greater than 0.3%, e.g., at least 0.5%, at least 1%, at least 2.5%, at least 5%, or more. In some embodiments the cloning efficiency is between 0.5% and 25%, or between 0.5% and 50%, or between 0.5% and 90%. Cloning efficiency may be determined, e.g., by plating cells in individual wells of microtiter plates at low density, such that many or most of the wells will contain no cells or single cells. In other embodiments cells are plated at slightly higher density, such that wells will contain on average 2 or 5 cells. In some embodiments cells are plated in larger dishes but at low density such that most or all cells are not in physical contact with other cells, e.g., so that they can form distinct colonies. Such colonies can later be isolated using methods known in the art. An ES cell colony may contain, e.g., at least 20, at least 30, at least 50, at least 100, or at least 200 ES cells.

In some embodiments pluripotent cells, e.g., ES cells, derived or cultured according to the invention exhibit increased amenability to genetic modification relative to pluripotent cells, e.g., ES cells, derived or cultured using standard techniques. For example, in some embodiments the frequency of generating transfected cells upon transfection is increased by at least a factor of 2, e.g., between 2- and 5-fold, or between 5- and 10-fold, or between 10- and 50-fold, or more, e.g., up to 100-fold, 500-fold, 1,000-fold, relative to the frequency when standard techniques of derivation or culture are used. In some embodiments the frequency of generating colonies of stably transfected cells in which transfected DNA has intergrated into the genome upon transfection is increased by at least a factor of 2, e.g., between 2- and 5-fold, or between 5- and 10-fold, or between 10- and 50-fold, or more, e.g., up to 100-fold, 500-fold, 1,000-fold, relative to the frequency when standard techniques of derivation or culture are used. some embodiments the cells exhibiting the improved properties are human ES cells. In some embodiments a human XX ES cell derived or cultured according to the present invention does not exhibit X-chromosome inactivation. In some embodiments a pluripotent cell line or cell culture is characterized in that most of the cells (e.g., at least 50%, 60%, 70%, 80%, or more) recover after freezing and thawing.

In some embodiments, a pluripotent cell or cell line cultured according to the invention, e.g., a human ES or iPS cell line exhibits capacity to develop into an even wider variety of cell types, e.g., in vitro, than when standard techniques of derivation or culture are used.

In some embodiments, a pluripotent non-human cell or cell line cultured according to the invention exhibits greater capacity to participate in formation of chimeras than when standard techniques of derivation or culture are used. In some embodiments, a pluripotent non-human cell or cell line cultured according to the invention exhibits greater capacity to contribute to the germline in chimeras than when standard techniques of derivation or culture are used.

In some embodiments, the methods and compositions of the invention find use for derivation or culture of totipotent cells (pluripotent cells that can produce extraembryonic tissues). In some embodiments, the methods and compositions of the invention find use for derivation or culture of multipotent cells, e.g., cells that can give rise to multiple different cell lineages but not all. Examples of multipotent cells are hematopoietic stem cells, neural stem cells, mesenchymal stem cells, etc.

Pluripotent cells, such as ES cells or iPS cells, can contain at least one/one or more genetic modification. A genetic modification refers to an alteration to the genome of a cell, which is engineered by the hand of man in the cell or an ancestor of the cell. Alternatively, such cells can be non-modified, i.e., they have not been altered to contain a genetic modification. Such cells can be used, e.g., to produce non-genetically modified animals or in cell therapy applications in which it is desired to use cells that are not genetically modified. Genetic modification often refers to modifications using genetic engineering technology rather than, for example, exposing a cell to a chemical such as a mutagenic compound that can alter DNA. The genetic modifications that can be present in pluripotent cells, e.g., ES cells or iPS cells can be, but are not limited to, transgenes (cDNA, genes or portions thereof), mutations (targeted or random), conditional mutations, targeted insertions of foreign genes, YAC and BAC sized transgenes, all or part of a chromosome, which may be from the same species as the animal from which the cell was derived or another species. Modifications can include physical knockout of all or a part of a gene, functional knockout of a gene, introduction of a functional gene and introduction of DNA or a gene portion that changes the function/level of expression of a gene present in the cell (e.g., a promoter, enhancer or repressor). In some embodiments a modification comprises introducing a construct that encodes a short hairpin RNA or antisense RNA. Modifications can also be present in the cells used to derive pluripotent cells when these cells are obtained from the embryo, tissue, or animal from which they are isolated. As used herein, genetically modified pluripotent cells encompasses (i) pluripotent cells derived from cells that comprise a genetic modification when obtained from an embryo, tissue, or animal; (ii) pluripotent cells derived from cells that are genetically modified after being isolated but before being used to derive a pluripotent cell; and (iii) pluripotent cells that are genetically modified after they are derived. Pluripotent cells can have one or more genetic modifications, which can be consecutive genetic modifications or simultaneous. Modifications can all be of the same type (e.g., all introduction of exogenous DNA) or of more than one type (e.g., introduction of exogenous DNA, gene knockout and conditional gene knockout). They can also be a combination of modifications present in cells as isolated and alterations made after they are isolated, e.g., after being used to derive a pluripotent cell. The modifications made in genomic DNA can be chosen to produce a phenotype that is similar to (mimics) a condition that occurs in other species (e.g., humans) and the resulting non-human mammal, e.g., mouse, can thus serve as a model for that condition. In other embodiments a modification can be chosen to modify a phenotypic characteristic of an animal. In some embodiments a genetic modification “repairs” a mutation or defect, e.g., by homologous recombination. In some embodiments a genetic modification introduces a gene that encodes a therapeutically beneficial protein or RNA (e.g., a short hairpin RNA or antisense RNA) or functionally inactivates a gene that encodes a deleterious protein (e.g., a mutated or activated oncogene). In some embodiments an iPS cell has a genetic modification arising from the reprogramming process, e.g., introduction of gene(s) encoding reprogramming factor(s). In some embodiments an iPS cell does not have a genetic modification arising from the reprogramming process (e.g., the iPS cell was derived without genetic modification or such genetic modification has been excised from the genome) but has at least one genetic modification of interest not arising from the reprogramming process. In some embodiments an iPS cell has no genetic modifications. As used herein, a “genetic modification of interest” is often a genetic modification that introduces or modifies at least part of a gene other than a gene encoding a reprogramming factor.

A variety of methods, known to those of skill in the art, can be used to genetically modify cells derived, cultured, or used in accordance with the present invention. For example, an appropriate vector (e.g., a plasmid) can be used to introduce DNA into cells in order, for example, to integrate DNA into genomic DNA, express introduced DNA in recipient cells, cause recombination (homologous or nonhomologous) between introduced DNA and endogenous DNA or knock out endogenous gene(s), such as by means of the Cre/lox or Frt/flp method. Alternatively, alterations or mutations can be produced by chemical methods or radiation. Gene targeting can also be used to produce genetically modified pluripotent cells. See, Rideout, W. M. et al. Nature Genetics 24, 109, 2000, and references therein.

The invention contemplates a variety of uses for the pluripotent cells, cell lines, and animals derived, cultured, or generated as described herein. In general, pluripotent cells may be used for any purpose contemplated in the art for use of pluripotent cells, e.g., ES or iPS cells. See, e.g., PCT/US2008/004516 (WO/2008/124133) and PCT/US2008/010249 (WO/2009/032194).

In some embodiments a pluripotent cell derived or cultured according to the invention is used to produce one or more differentiated cells. Such cells are aspects of the invention. The cells could be, e.g., multipotent stem cells or fully differentiated cells. The cells may be, e.g., hematopoietic cells (e.g., of the myeloid or erythroid lineage), neural cells (e.g., neural precursors, neurons or glial cells), myoblasts, myocytes, cardiomyocytes, melanoblasts, keratinocytes, chondroblasts, chondrocytes, osteoblasts, osteoclasts, pancreatic beta cells, retinal cells, etc. Protocols known in the art for differentiating cells into cells of a desired type may be used. In some embodiments cells are used to generate a tissue or organ in vitro or to supplement a tissue or organ in vivo.

A pluripotent cell can be derived from a cell obtained from an individual of interest. The individual can be, e.g., a human suffering from a disease or condition. In some embodiments the disease is a neurodegenerative disease, e.g., Parkinson's disease, Alzheimer's disease, or amyotrophic lateral sclerosis. In some embodiments the individual suffers from diabetes. In some embodiments the individual suffers from heart failure or a muscle disorder. In some embodiments the disease is a heritable disease. In some embodiments the disease is sporadic, i.e., there is no evident pattern of inheritance. In some embodiments the individual has suffered an injury, e.g., traumatic brain injury, spinal cord injury. In some embodiments the individual is in need of cell therapy.

In some embodiments a pluripotent cell derived or cultured according to an inventive method is used to prepare a composition for cell therapy to be administered to a vertebrate subject, e.g., a non-human animal, or a human. In some embodiments the composition comprises autologous cells. In other embodiments the composition comprises non-autologous cells. In some embodiments the cells are genetically matched to an individual.

The invention provides methods of producing non-human vertebrates, e.g., non-human mammals, which can be genetically modified or non-genetically modified, using pluripotent cells, e.g., pluripotent ES cells or iPS cells of the invention. Such non-human vertebrates are aspects of the invention. In some embodiments the non-human vertebrates are mice. In some embodiments, non-human mammals are produced using methods known in the art for producing non-human mammals from ES or iPS cells. For example, ES or iPS cells are introduced into a blastocyst of the same species which is transferred to a suitable foster mother (e.g., a pseudopregnant female of the same species), under conditions suitable for production of live offspring. If a diploid blastocyst is used, chimeric offspring may be produced, which are typically derived in part from the ES cell or iPS cell and in part from the blastocyst into which the cell was introduced. Chimeric offspring may be interbred to generate homozygous animals if the chimeric offspring contain ES-derived contribution to the germ line as known in the art, In some embodiments, the mice are produced using methods that do not require production of chimera or chimeric offspring. In some embodiments pluripotent ES cells are introduced into tetraploid blastocysts of the same mammalian species under conditions that result in production of an embryo (at least one/one or more embryo) and the resulting embryo(s) transferred into an appropriate foster mother, such as a pseudopregnant female of the same mammalian species. The resulting female is maintained under conditions that result in development of live offspring, thereby producing a non-human mammal derived from the introduced ES cells. See, e.g., U.S. Pat. No. 6,784,336. In some embodiments, the mouse is produced by a method that involves laser-assisted injection or piezo-injection of ES cells of the invention into four- or eight-cell embryos. In some embodiments the mouse is produced without need to generate a chimera, e.g., using methods described in PCT/EP2003/002180 (WO/2003/073843), Another embodiment of the present invention is a method of producing a non-human mammalian strain, such as a mouse strain, e.g., a genetically engineered mouse strain, that is derived from a given (single) iPS or ES cell clone of the present invention without outcrossing with a wildtype partner. See, e.g., U.S. Pat. No. 6,784,336. In some embodiments the mice are genetically modified, e.g., they are derived from an ES or iPS cell that is genetically modified. The invention contemplates interbreeding non-human vertebrates, e.g., mice, derived from the ES cells or iPS cells with mice of any strain of interest, the resulting strains being aspects of the invention.

The non-human vertebrates of the invention, such as mice, can be used for a wide variety of purposes. In some embodiments, a non-human vertebrate is used as a model for a condition in order to facilitate study of the condition. In some embodiments, a non-human vertebrate is used as a model for a condition for which a preventive or therapeutic drug is sought. In one embodiment, a method of identifying a drug to be administered to treat a condition in a mammal comprises producing, using a pluripotent cell of the present invention and/or a method of the present invention, a non-human vertebrate, e.g., a mouse, that is a model of the condition; administering to the non-human vertebrate a drug, referred to as a candidate drug, to be assessed for its effectiveness in treating or preventing the condition; and assessing the ability of the drug to treat or prevent the condition. If the candidate drug reduces the extent to which the condition is present or progresses or causes the condition to reverse (partially or totally), the candidate drug is a drug to be administered to treat the condition. In one embodiment, the condition is insulin-dependent diabetes mellitus and the non-human vertebrate is a genetically engineered NOD mouse.

In some embodiments methods and compositions are used to derive or culture pluripotent cells from endangered or threatened species or commercially significant species, e.g., domesticated species, e.g., pets or farm animals, and/or to generate animals from such cells. As used herein, a “commercially significant species” is one whose cultivation or sale contributes at least $100,000 to the gross domestic product (GDP) of at least one country and/or that is an object of interstate or inter-country commerce or tourism. As used herein, an “endangered or threatened species” is one listed as being endangered or threatened by a government agency responsible for making such listings.

Those skilled in the art will recognize, or be able to ascertain using no more than routine experimentation, many equivalents to the specific embodiments of the invention described herein. The scope of the present invention is not intended to be limited to the Description or the details set forth therein. Articles such as “a”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the claims (whether original or subsequently added claims) is introduced into another claim (whether original or subsequently added). In particular, any claim that is dependent on another claim can be modified to include one or more elements or limitations found in any other claim that is dependent on the same base claim. Furthermore, where the claims recite a composition, the invention provides methods of making the composition, e.g., according to methods disclosed herein, and methods of using the composition, e.g., for purposes disclosed herein. Also, where the claims recite a method of making a composition, the invention provides compositions made according to the inventive methods and methods of using the composition, unless otherwise indicated or unless one of ordinary skill in the art would recognize that a contradiction or inconsistency would arise.

Where elements are presented as lists, e.g., in Markush group format, each subgroup of the elements is also disclosed, and any element(s) can be removed from the group. For purposes of conciseness only some of these embodiments have been specifically recited herein, but the invention includes all such embodiments. It should also be understood that, in general, where the invention, or aspects of the invention, is/are referred to as comprising particular elements, features, etc., certain embodiments of the invention or aspects of the invention consist, or consist essentially of, such elements, features, etc.

Where numerical ranges are mentioned herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. Where phrases such as “less than X”, “greater than X”, or “at least X” is used (where X is a number or percentage), it should be understood that any reasonable value can be selected as the lower or upper limit of the range. It is also understood that where a list of numerical values is stated herein (whether or not prefaced by “at least”), the invention includes embodiments that relate to any intervening value or range defined by any two values in the list, and that the lowest value may be taken as a minimum and the greatest value may be taken as a maximum. Furthermore, where a list of numbers, e.g., percentages, is prefaced by “at least”, the term applies to each number in the list. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments 5% or in some embodiments 10% of a number in either direction (greater than or less than the number) unless otherwise stated or otherwise evident from the context (e.g., where such number would impermissibly exceed 100% of a possible value).

In addition, any particular embodiment(s), aspect(s), element(s), feature(s), etc., of the present invention, e.g., any compound, animal species, or strain, may be explicitly excluded.

### EXEMPLIFICATION

### Example 1

### Metastable Pluripotent States in NOD Mouse Derived ES Cells

Embryonic stem (ES) cells are isolated from the inner cell mass (ICM) of blastocysts, whereas epiblast stem cells (EpiSCs) are derived from the post-implantation epiblast and display a restricted developmental potential. In this example, pluripotent states are characterized in the non-obese diabetic (NOD) mouse strain, which prior to this study was considered “non-permissive” for ES cell derivation. The results reveal that NOD stem cells can be stabilized by providing constitutive expression of Klf4 or c-Myc or small molecules that can replace these factors during in vitro reprogramming. The NOD ES and iPS cells appear “metastable”, as they acquire an alternative EpiSC-like identity after removal of the exogenous factors, while their reintroduction converts the cells back to ICM-like pluripotency. These findings suggest that stem cells from different genetic backgrounds can assume distinct states of pluripotency in vitro, the stability of which is regulated by endogenous genetic determinants and can be modified by exogenous factors. Complete reference citations for this example appear immediately following the text of the example.

Mouse embryonic stem cells (ESC), isolated from the inner cell mass (ICM) of blastocysts, can be propagated in vitro in an undifferentiated state and recapitulate all defining features of ICM cells (Jaenisch and Young, 2008). ICM-derived ES cells, when used to generate chimeras, can contribute to all somatic cell lineages and to germ cells, maintain both X chromosome alleles in a reactivated state, but are unable to contribute to the trophectoderm lineages (Rossant, 2008). Mouse ES-like cells derived either by somatic cell nuclear transfer or by direct in vitro reprogramming (termed induced pluripotent stem (iPS) cells) share all of these defining features (Hanna et al., 2008; Takahashi and Yamanaka, 2006; Wakayama et al., 1998; Wernig et al., 2007).

Despite their apparent common origin from the ICM and their ability to propagate in vitro while maintaining pluripotency, human ES cells are phenotypically and functionally distinct from mouse ES cells (Thomson et al., 1998). Human ES cells require different growth conditions and rely on bFGF and ActivinA/TGFbeta signaling to maintain their pluripotent state, whereas mouse ES cells require Lif/Stat3 and Bmp4 signaling (Xu et al., 2005; Ying et al., 2003). Human ES cells differ epigenetically from mouse ES cells by several criteria such as X chromosome inactivation and pluripoteney factor promoter occupancy across the genome (Boyer et al., 2005; Silva et al., 2008b; Tesar et al., 2007). Recently pluripotent cells from the epiblast of post-implantation murine embryos termed Epiblast stem cells (EpiSCs) have been isolated and found to recapitulate defining features of human stem cells (Brons et al., 2007; Tesar et al., 2007). EpiSC and human ES cells share the flattened morphology, intolerance to passaging as single cells, dependence on Activin/Nodal signaling, inactivation of the X chromosome in female cell lines, and the ability of some of the isolated EpiSC lines to differentiate into trophectoderm. In contrast to mouse ES cells, EpiSCs are extremely inefficient to generate chimeras and are unable to contribute to the germline (Brons et al., 2007; Guo et al., 2009; Tesar et al., 2007). The similarities between human ES cells and mouse EpiSCs have provoked questions concerning the nature, origin and the in vivo counterpart of human ES cells during normal development (Lovell-Badge, 2007).

Recent advances provided by new genetic and chemical approaches to isolate stem cells have enabled the derivation of iPS cells and ICM- or Epiblast-derived stem cells from different species. For instance, while EpiSCs can be generated from “non-permissive” species such as rat, ES cells cannot be established from the rat ICM under the same conditions used to isolate mouse ES cells (Brons et al., 2007). Moreover, whereas rat ICM-derived ESCs or iPS cells generated via transduction of Oct4, Sox2, Nanog and Lin28 can only bc propagated in the presence of glycogen synthase kinase 3 (GSK3) and mitogen-activated protein kinase pathway (ERK) inhibitors (21 conditions) (Li et al., 2008; Li et al., 2009; Liao et al., 2009), rat iPS cells generated with lentiviruses encoding Oct4, Sox2, Klf4 and c-Myc could be propagated like mES cells without these inhibitors (Liao et al., 2009). Moreover, EpiSCs can be converted into ES cells by over expressing KLF4 and growing the cells in 21 conditions (Guo et al., 2009). These findings raise fundamental questions relating to pluripotency.

To address some of these questions, work described in this example utilized the non-obese diabetic (NOD) mouse strain as a model to characterize different isolated pluripotent states, as this strain is non-permissive for the derivation of ES cells, but allows the isolation of EpiSCs from day E6.5 embryos (Brons et al., 2007). The results showed that continuous ectopic expression of Klf4 or c-Myc transcription factors is sufficient for derivation of ICM-like pluripotent NOD iPS and ES cells. Supplementing mouse ES cell growth conditions with small molecules known to replace the function of Klf4 and c-Myc during iPS cell generation can facilitate the derivation of germline competent NOD ES cells. Importantly, upon removal of the exogenous factors that help maintain the ICM-like cell state, the NOD pluripotent cells adapted an alternative Epiblast-like pluripotent state and were functionally and molecularly similar to EpiSCs. These results provide an example in which failure to stabilize ICM-like pluripotency from a non-permissive strain can lead to the attainment of an in vitro acquired alternative pluripotent state. Moreover, these findings support the notion that appropriate growth conditions may have not yet been devised to allow in vitro stabilization of ICM-like pluripotent cells from other species than mouse.

Results

Derivation of Transgene-Dependent iPS Cells from NOD Mice

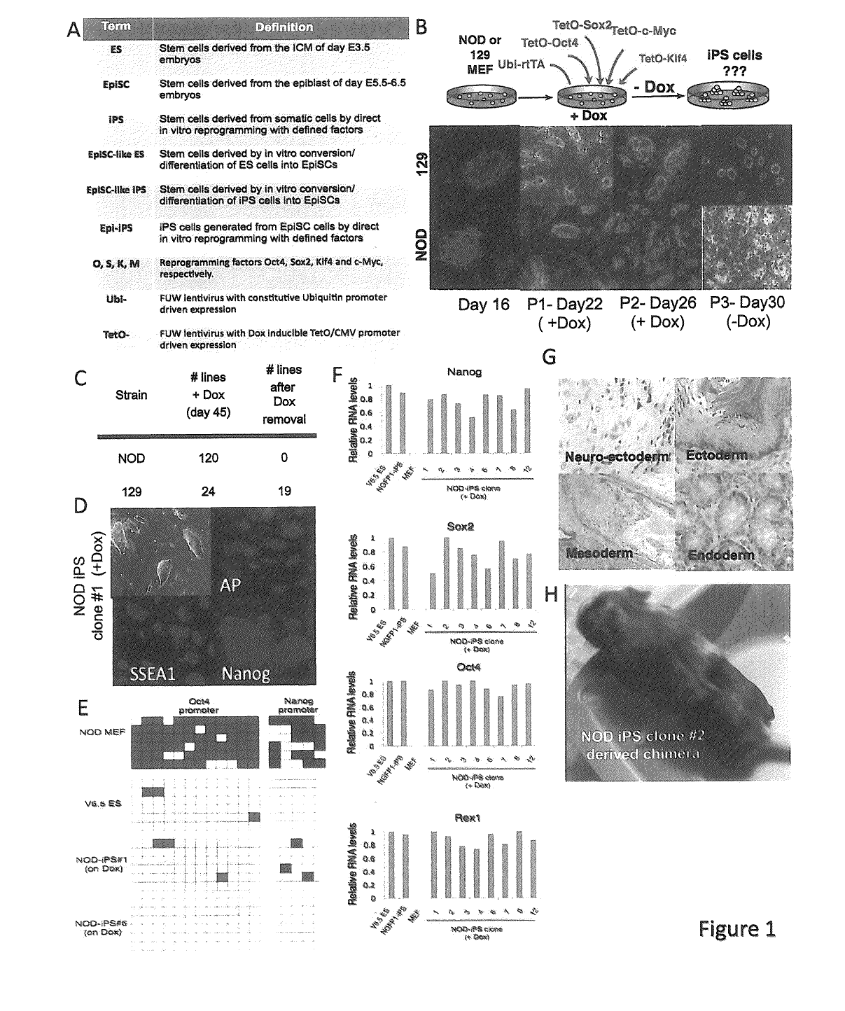

An attempt was made to derive NOD iPS cells by infecting mouse embryonic fibroblasts (MEFs) with doxycycline (Dox)-inducible lentiviral vectors encoding the four reprogramming factors Oct4, Sox2, Klf4 and c-Myc (see FIG. 1A for definitions) and a constitutively active lentivirus encoding the reverse tetracycline transactivator (Ubi-M2rtTA) (FIG. 1B). The infected NOD and control 129 MEFs were cultured in the presence of Dox and colonies appeared 12 days after Dox induction, many of which acquired ES-like morphology. However, unlike the 129-derived iPS cells that could be maintained in the absence of Dox after 20-30 days, the NOD ES-like cells differentiated upon Dox withdrawal (FIG. 1B). To analyze individual iPS-like clones, colonies were picked from transduced NOD-MEF cultures at day 16 and further passaged in the presence of Dox for up to 45 days. After Dox withdrawal, all 120 Nanog+NOD-iPS colonies underwent differentiation, while the majority of 129-derived iPS lines remained undifferentiated (FIG. 1C). The Dox-dependent NOD-iPS clones had all the characteristics of pluripotent ES cells such as expression of ES cell markers, demethylation of the endogenous Oct4 and Nanog promoters and reactivation of endogenous pluripotency genes (FIG. 1 D-F). Finally, the Dox-dependent NOD-iPS cells generated differentiated teratomas and adult chimeras (FIG. 1 G-H). These results suggest that the four factors are capable of inducing a pluripotent ES-like state in NOD somatic cells. However, in contrast to iPS cells from the 129 strain, the NOD pluripotent state was unstable requiring the continuous expression of the reprogramming factors.

Constitutive Ectopic Expression of Klf4 or c-Myc Facilitates Derivation of NOD ES and iPS Cells

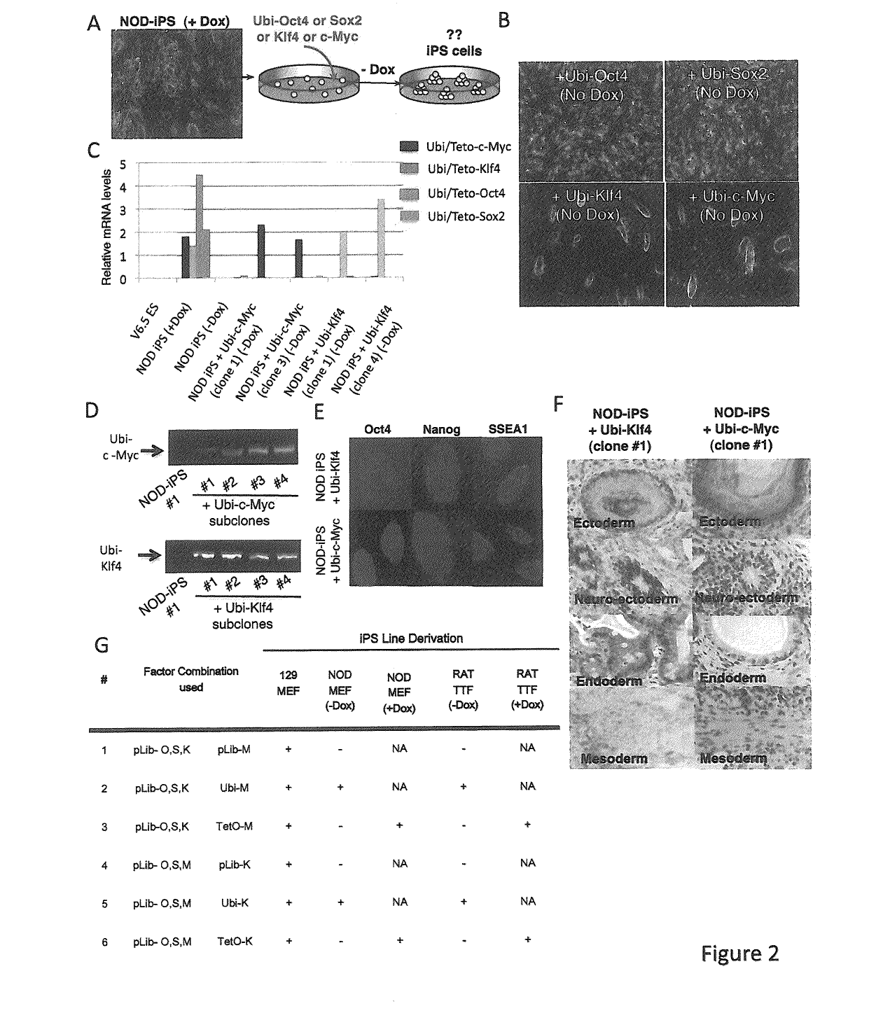

To define the exogenous factors required to stabilize pluripotency in the NOD background, Dox-dependent iPS cells were transduced with constitutively expressed lenti-viruses encoding reprogramming factors and the ability of the cells to propagate was tested after Dox withdrawal (FIG. 2A). Constitutive expression of Klf4 or c-Myc, but not Oct4 or Sox2, enabled the derivation of Dox-independent NOD iPS clones (FIG. 2B). All Dox-independent NOD iPS cells carried the Ubi-c-Myc and Ubi-Klf4 proviruses and expressed transgene encoded transcripts (FIG. 2C-D). The NOD iPS cells stained positive for pluripotency markers and generated teratomas (FIG. 2E-F). Factor transduction by retroviral vectors, which are silenced in ES and iPS cells (Jahner et al., 1982), failed to yield any iPS cells from NOD mice, consistent with the requirement for continuous expression of c-Myc and Klf4 (FIG. 2G). Conversely, iPS cells were readily isolated from NOD fibroblasts by using a combination of retroviruses encoding OSM factors and a constitutive virus expressing Klf4, or a combination of retroviruses encoding OSK factors and a constitutively expressed lentivirus expressing c-Myc (FIG. 2G). When Dox inducible lentiviruses encoding c-Myc or Klf4 were used in the latter combinations instead of the constitutively expressed lentiviruses, NOD iPS lines could only be grown in the presence of Dox (FIG. 2G). The derivation of iPS cells from rat fibroblasts required similar conditions (FIG. 2G).

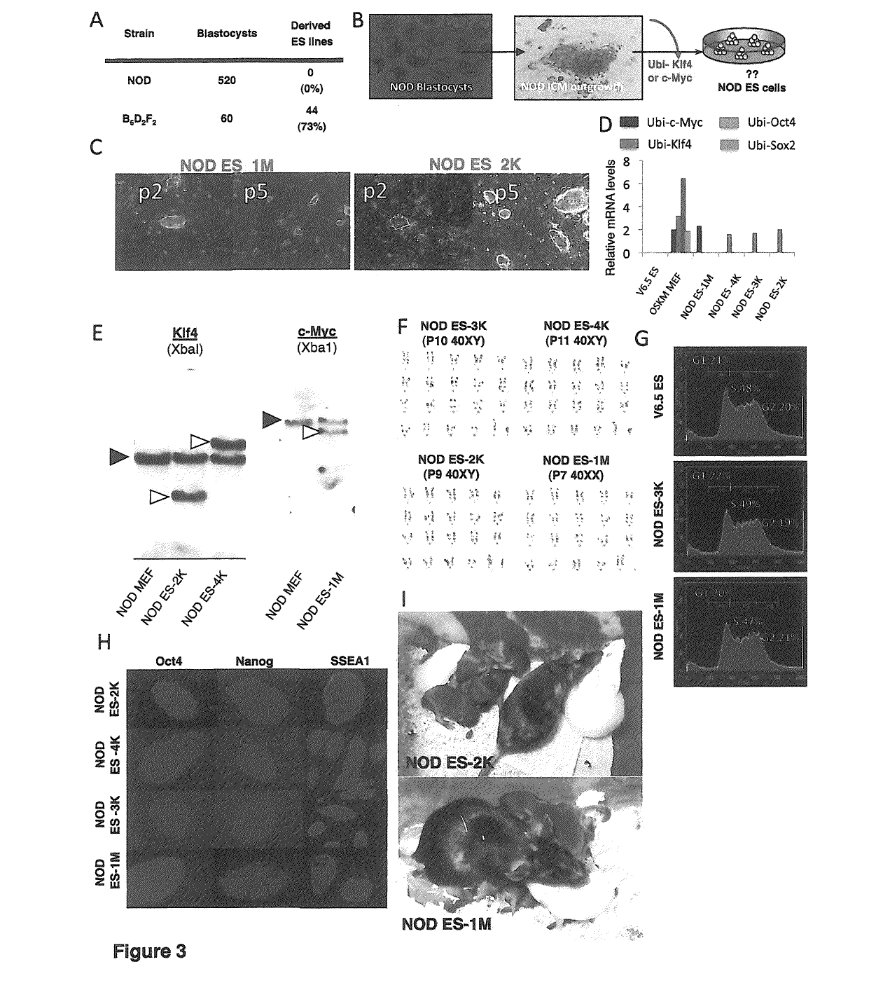

Additional work tested whether constitutive expression of c-Myc and/or Klf4 would allow derivation of ES cells from NOD ICMs. NOD blastocysts failed to yield any ES cells under conditions routinely used for ES cell derivation (FIG. 3A). While NOD-ICM outgrowths were obtained after plating on feeders, the cells did not survive passaging (FIG. 3B). However, when NOD ICM outgrowths were infected with constitutive lenti-viruses encoding Klf4 or c-Myc the cultures could be dissociated and maintained in mouse ES cell conditions (defined as mESM: DMEM supplemented with fetal bovine serum (FBS) and Lif grown on irradiated feeders) resulting in the generation of independent NOD ES cell lines carrying either c-Myc or Klf4 proviruses (FIGS. 1A, 3E). Southern blot and RT-PCR analysis verified proviral integration and transgene-specific transcripts in the isolated lines (FIG. 3 D-E). The NOD-ES lines had a normal karyotype and a cell cycle pattern identical to control V6.5 ES cell, expressed pluripotency markers and generated adult chimeras with germ-line contribution (FIG. 3F-I and Table S1). Tumor formation was observed in some of the c-Myc transgenic NOD ES line derive chimeras and offspring (data not shown) probably as a result of the ectopic expression of the c-Myc oncogenic transgene (Okita et al., 2007).

### Generation of Genetically Unmodified Germline Competent NOD ES Cells

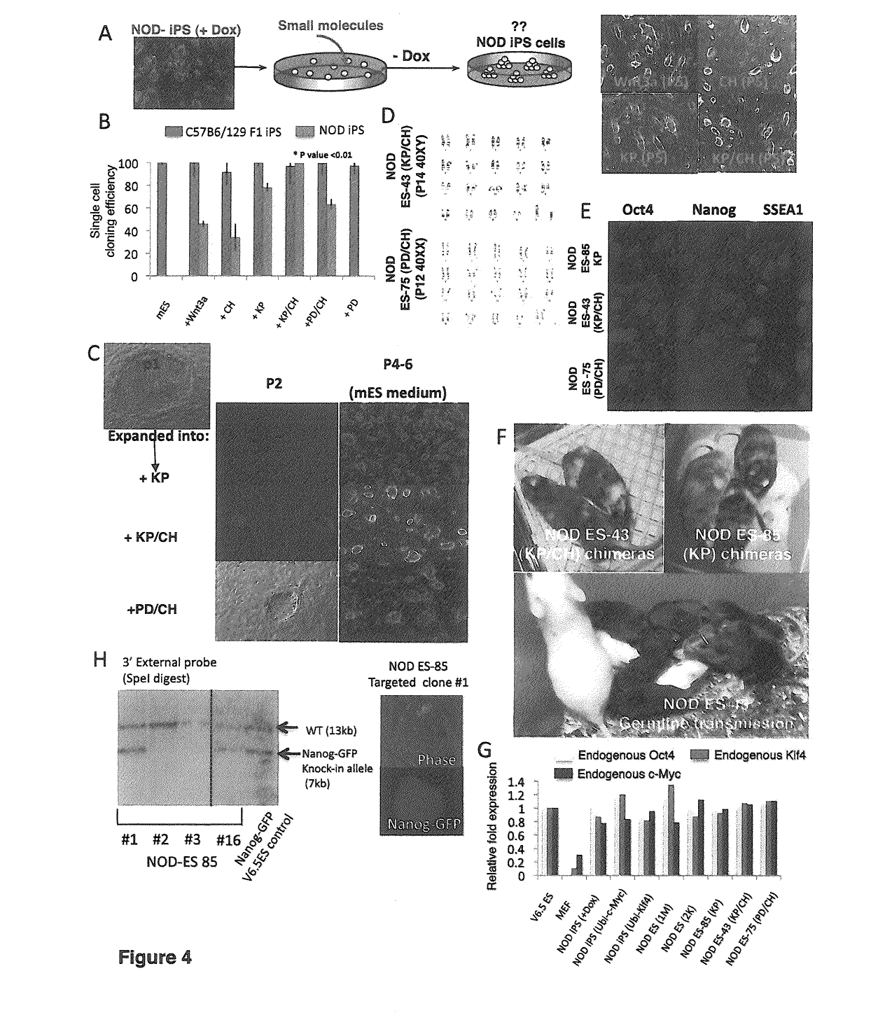

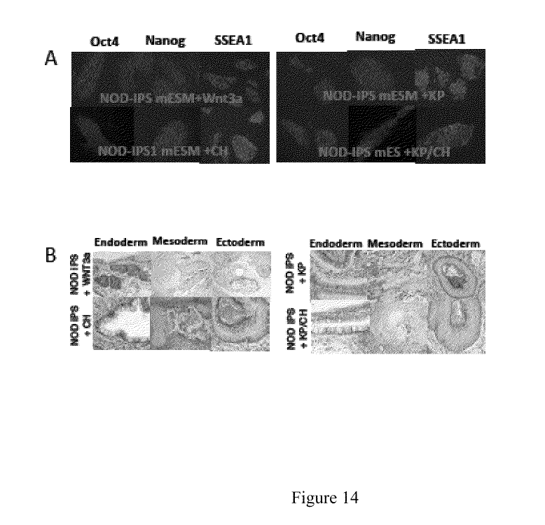

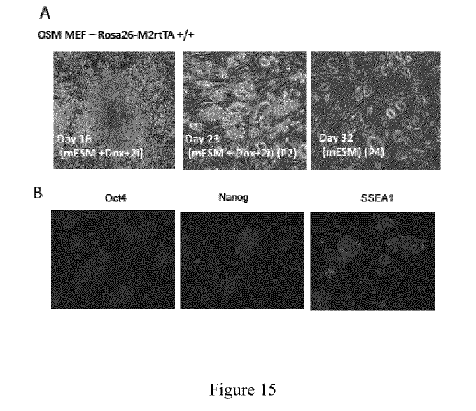

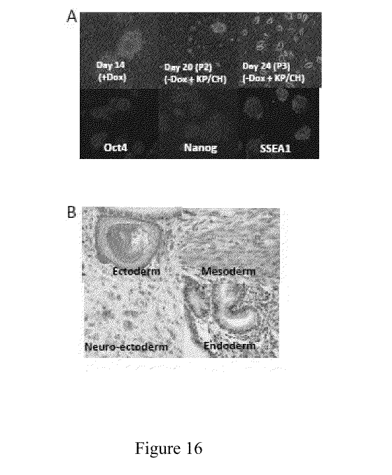

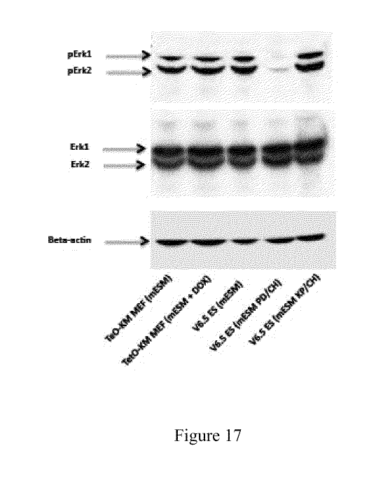

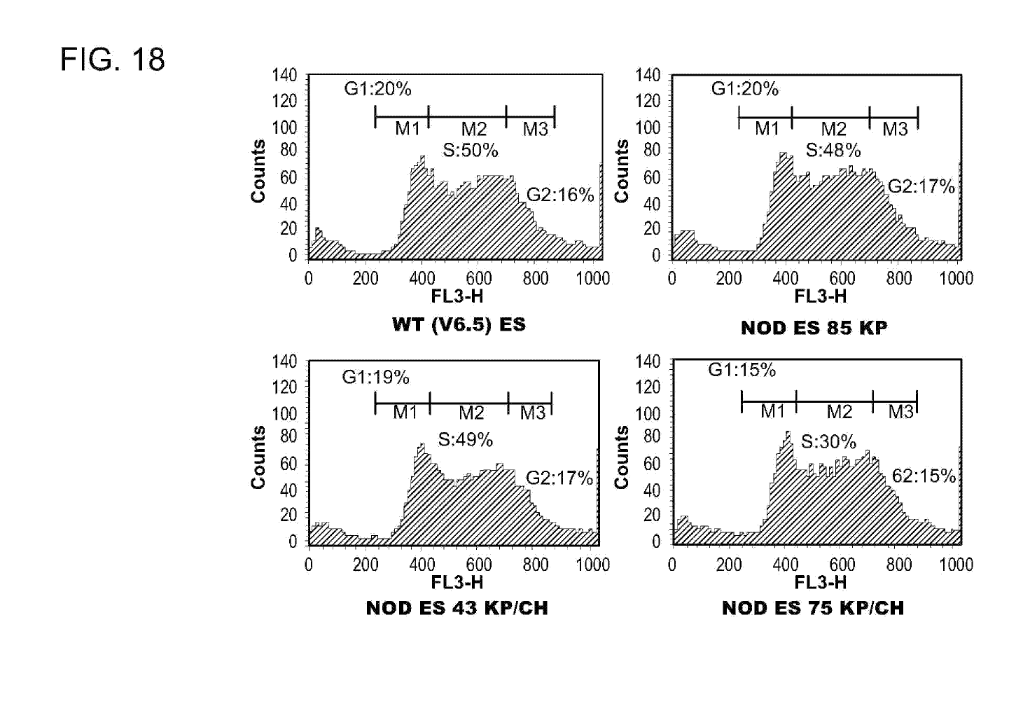

Small molecules have been identified to replace some of the reprogramming factors in iPS derivation (Huangfu et al., 2008; Shi et al., 2008). We have found that Wnt signaling promotes the derivation of iPS cells in the absence of c-Myc (Marson et al., 2008) and have identified Kenpaullone, a GSK3b and CDK1/cyclin B inhibitor, as a small molecule that replaces Klf4 during iPS reprogramming (Lyssiotis et al., 2009). We tested whether these compounds could replace constitutive Klf4 or c-Myc expression in propagating NOD iPS and ES cells. Dox-dependent NOD-iPS cells were grown in mESM lacking Dox but supplemented with: (1) Wnt3a, (2) the glycogen synthase kinase 3 inhibitor CHIR99021 (CH), or (3) Kenpaullone (KP). All three conditions supported Dox-independent growth of pluripotent NOD-iPS cells (FIGS. 4A and 14). Notably, it was recently reported that the “21” culture conditions, using the ERK-cascade inhibitor PD184352 (PD) and the GSK3 inhibitor CH or 6-bromo-indirubin-3′-oxime (BIO) facilitate the derivation of rat ES cells (Buehr et al., 2008; Li et al., 2008). Consistent with this observation, NOD iPS cells were readily propagated independently of Dox in the presence of 21, although at reduced single-cell cloning efficiency compared to mESM conditions supplemented with both KP and CH (KP/CH) (FIG. 4B). In a similar manner to KP, the combination of PD/CH replaced the requirement for ectopic expression of the Klf4 transgene during iPS generation (FIG. 15). Rat iPS cells could also be propagated in mESM with KP/CH (FIG. 16). Importantly, KP/CH did not inhibit ERK phosphorylation, suggesting that stabilization of pluripotency can occur in the absence of ERK inhibition (FIG. 17).

To derive NOD ES cells, E3.5 NOD blastocysts were plated on mouse feeders and grown under the optimized culture conditions supplemented with the different inhibitors (FIG. 4C). 16 lines were isolated in mESM containing KP (NOD-ES#85), KP/CH(NOD-ES#43), or PD/CH(NOD-ES#75). The ESCs remained stable in culture but required the continuous presence of the defined inhibitors, expressed pluripotency markers and retained a normal karyotype (FIG. 4D-E and 14-15). The cells generated adult chimeras (FIG. 4F) and contributed to the germ line (FIG. 4F and Table S1). Importantly, the NOD ES or iPS cells maintained with exogenous factors expressed endogenous levels of Klf4 and c-Myc similar to those observed in control 129 ES cells (FIG. 4G). This argues that the inability of NOD cells to stabilize pluripotency in the absence of the exogenous factors is not due to failure to reactivate the endogenous Klf4 or c-Myc genes. Finally, additional work tested whether NOD ES cells could be targeted by homologous recombination. NOD-ES#85 cells were electroporated with a linearized Nanog-GFP knock-in targeting construct (Hatano et al., 2005), followed by puromycin selection. Resistant colonies were picked after 10 days of drug selection and 2 out of 16 colonies analyzed demonstrated correct targeting of the Nanog locus (FIG. 4H).

### Destabilized NOD ICM-Like Stem Cells Adapt an Alternative Epiblast-Like Pluripotent State

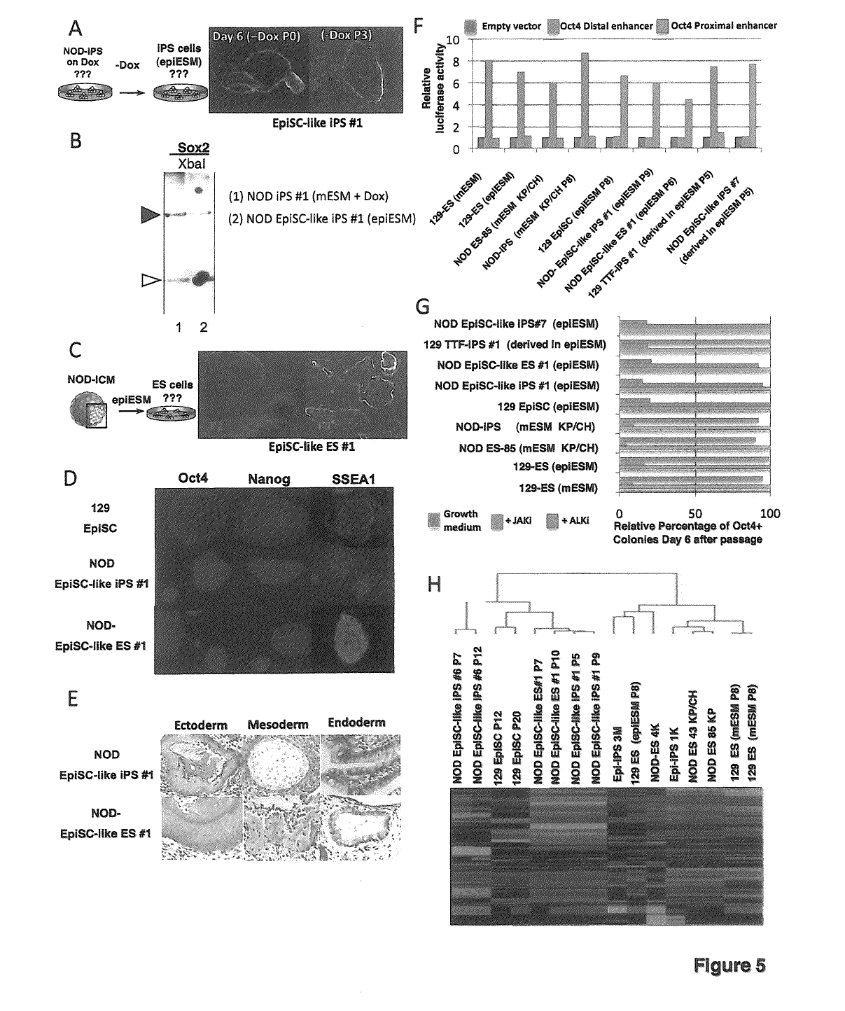

The NOD stem cells stabilized by ectopic expression of Klf4 or c-Myc or the presence of small molecules displayed ICM-like pluripotency as evident by their ability to generate chimeras with germline contribution. Upon plating these cells after withdrawal of the exogenous stimuli (Dox or inhibitors), we noticed distinct colonies with flat morphology (˜0.3% of plated cells) (FIG. 5A). These colonies could not be propagated following dissociation into single cells (by trypsinization), typically used to passage mouse ES cells, but could be stably propagated by passage of smaller clumps using collagenase or mechanical dissociation in the presence of bFGF (DMEM/F12 supplemented with FBS and bFGF, referred to as epiESM). The flat colonies were morphologically distinct from mouse ES cells and had a similar morphology to EpiSC cells and were termed EpiSC-like iPS cells (FIG. 1A). Moreover, the NOD EpiSC-like iPS line #1 and the Dox dependent NOD-iPS #1 cells from which the former line was derived, carried an identical Sox2 genetic integration pattern, thus excluding contamination as a source for the EpiSC-like cells (FIG. 5B).

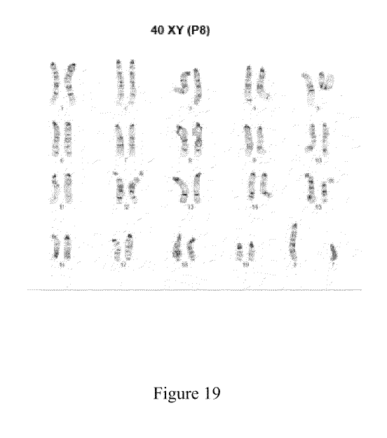

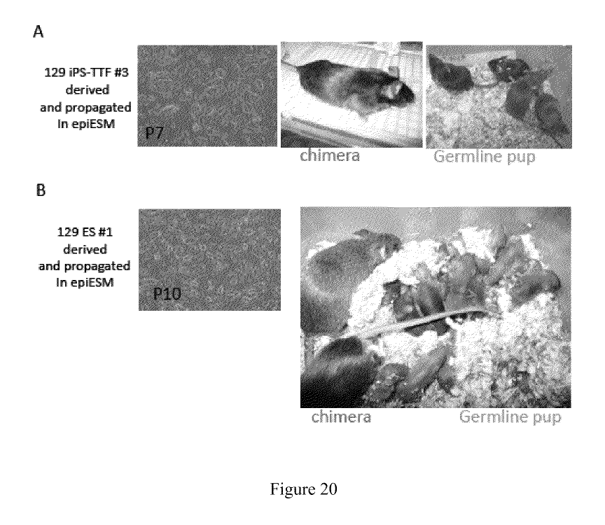

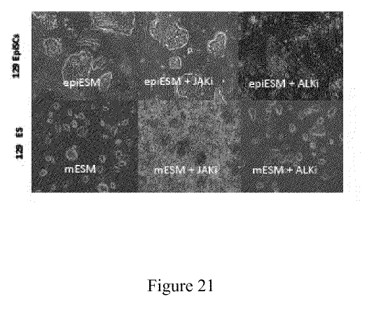

NOD EpiSCs have been previously derived from the epiblast of day E6.5 developing embryos (Brons et al., 2007). This prompted further testing with respect to whether EpiSC-like cells can also be isolated from NOD ICM in the absence of exogenous factors. NOD day E3.5 blastocysts were explanted and ICM outgrowths were manually dissociated after plating in epiESM derivation medium (see experimental procedures) and propagated in epiESM, resulting in stable lines termed EpiSC-like ES cells (FIGS. 1A, 5C and 19). NOD EpiSC-like iPS and NOD EpiSC-like ES cell lines (see FIG. 1A) derived by the different approaches expressed pluripotency markers, were not capable of generating chimeric mice, but were pluripotent as evident by their ability to generate teratomas (FIG. 5D-E and Table S1) (Brons et al., 2007; Guo et al., 2009; Tesar et al., 2007). In contrast, when the same derivation protocol was used on 129 embryos, typical murine ES cells were generated that could be passaged by trypsinization, were dependent on Lif/Stat3 signaling for their self renewal and were capable of generating adult chimeras with germline transmission (FIG. 20 and Table S2).

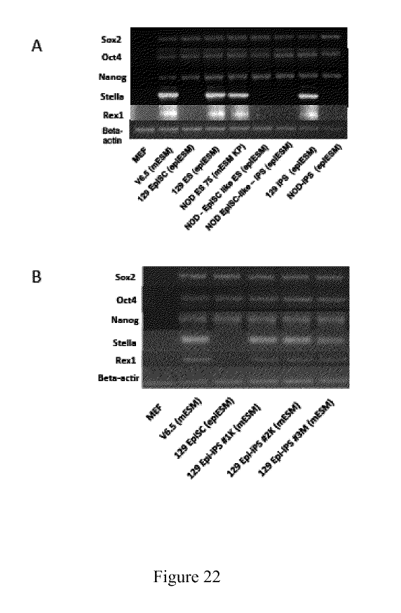

NOD-derived ES and EpiSC-like cells were characterized in a series of assays: (i) Cells were transfected with a luciferase reporter construct under the control of either the distal or the proximal enhancers that control expression of the Oct4 gene in the mouse ICM and Epiblast, respectively (Tesar et al., 2007). The NOD EpiSC-like ES and iPS lines preferentially utilized the Oct4 proximal enhancer similar to EpiSCs (FIG. 5F). In contrast, NOD ES and iPS cells grown in mESM with KP/CH, as well as controls including 129 mES or iPS cells derived and grown in mESM or epiESM conditions, utilized the distal enhancer consistent with the notion that these cells resemble mouse ES cells (Tesar et al., 2007). (ii) Lif-dependent self-renewal: Mouse ES cells are dependent on Lif signaling and readily differentiate when exposed to JAK inhibitor (JAKi) that blocks Stat3 phosphorylation. In contrast, mouse EpiSCs or human ESCs rely on activin A (Inhb)/Nodal signaling to maintain pluripotency and rapidly differentiate in the presence of ALKi, an inhibitor of type I activin receptor-like kinases (Tesar et al., 2007). We exposed ESCs and EpiSCs to either ALKi or JAKi to test which of these pathways controls their self-renewal. NOD ICM derived ESCs maintained in mESM KP/CH conditions were dependent on Lif/Stat3 signaling in contrast to NOD EpiSC-like cells, which required Activin A/Nodal signaling (FIG. 5G). (iii) Gene expression: EpiSCs and ESCs display different developmental potentials and several key pluripotency genes are differentially expressed in the two cell types (Tesar et al., 2007). Unbiased clustering of global gene expression profiles demonstrated that the NOD EpiSC-like ES and iPS lines clustered closely with EpiSCs, and were distinct from 129 ICM derived ES grown in mESM or epiESM conditions (FIGS. 5H and 22). Our results support the notion that, in spite of the presence of Lif and other growth factors present in the serum or provided by the feeders, NOD stem cells in vitro adapt a pluripotent state that highly resembles EpiSCs when the exogenous components that maintain their ICM-like pluripotent state are removed. In contrast, 129 ES cells maintained their ICM-like pluripotency in epiESM conditions and were dependent on Lif/Stat3 pathway stimulated by signals originating from the FBS and feeders used in these growth conditions. Consistent with this, differentiating 129 ES cells into EpiSC-like state was achieved only upon removal of FBS and Lif, and required prolonged culturing of the cells in serum free N2B27 defined medium supplemented with high levels of bFGF and Activin A (data not shown, FIG. 20 and (Guo et al., 2009)).

### Pluripotent States of NOD Stem Cells are Unstable and are Affected by Exogenous Factors

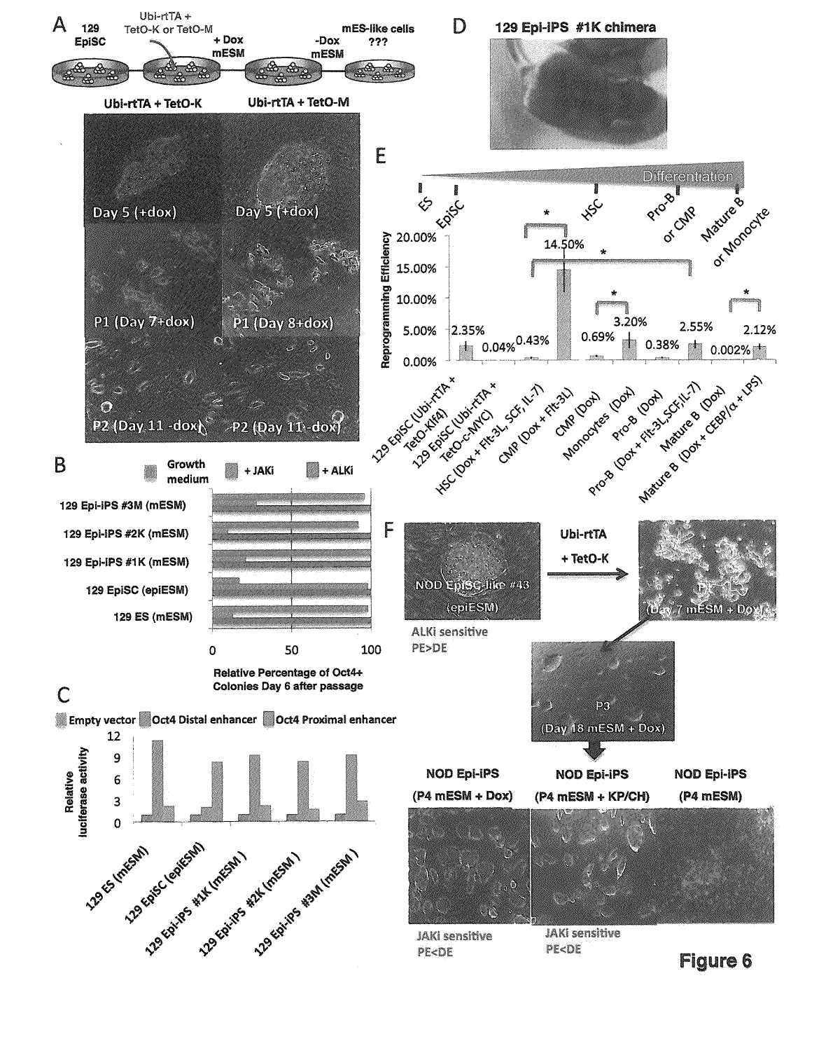

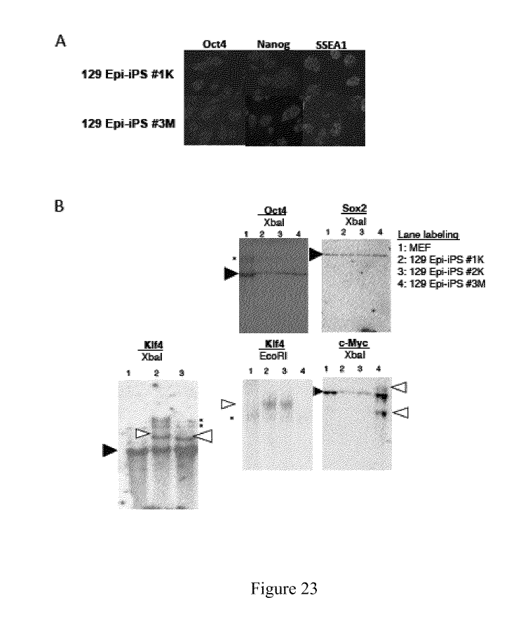

Work described here further analyzed whether the exogenous stimuli defined in this study could interconvert the two distinct pluripotent states. This work first tested whether infection of previously characterized E5.5 derived 129 EpiSCs (Tesar et al., 2007) with a Klf4 or c-Myc Dox inducible vectors could convert the EpiSCs to an ICM-like pluripotent state. Consistent with other reports, EpiSCs did not spontaneously convert into ES-like cells upon culturing in mESM, but rather differentiated (Guo et al., 2009; Tesar et al., 2007). Infected EpiSC cultures maintained in mESM with Dox gave rise to distinguishable small round colonies (FIG. 6A). When trypsinized into single cells and propagated on MEFs, they eventually acquired typical mouse ES-like morphology. Dox was withdrawn after 7-12 days and clonal lines termed Epi-iPS cells (FIG. 1A and (Guo et al., 2009)) were stable and morphologically indistinguishable from mouse ES cells (FIG. 6A). Southern analysis verified the presence of c-Myc or Klf4 proviral integrations (FIG. 23). Moreover, unlike their donor EpiSCs, Epi-iPS cells grew stably in mESM conditions and their pluripotency was disrupted by inhibition of Stat3, but not of activin/nodal pathway (FIG. 6B). Epi-iPS cells showed preferential utilization of the Oct4 distal enhancer similarly to mESCs (FIG. 6C). Epi-iPS cells were transcriptionally indistinguishable from 129 ICM-like ES cells and did not cluster with the EpiSCs (FIGS. 5H and 22). High contribution chimeras were derived from Epi-iPS cells, verifying that ICM-like pluripotency had been re-established (FIG. 6D). A recent report (Guo et al. 2009) showed that Klf4 over-expression as well as growth in PD/CH was required to convert EpiSCs into iPS cells. In contrast, we found that such conversion can be accomplished either by over expression of the Klf4 or the c-Myc transcription factor, or by supplementing the medium with KP or KP/CH or PD/CH, consistent with the ability of these small molecules to replace Klf4 or c-Myc during iPS cell generation (FIG. 6A-D, 15, 23-24). It should be emphasized that only a small fraction of Klf4 or c-Myc transduced EpiSC cells (up to 2%) can be reprogrammed back to ICM-like pluripotency (FIG. 6E and (Guo et al., 2009)). However, the efficiency of converting EpiSCs into iPS cells was within the same range as reprogramming efficiencies observed of hematopoietic cells at various differentiation stages (FIG. 6E). These findings indicate that expression of OSKM transcription factors can induce ICM-like pluripotency in different somatic cells and that the efficiency of reprogramming is not dictated by the differentiation state of the donor cell but rather depends on additional parameters such as cytokine stimulation, cell cycle state, enhancement by additional reprogramming factors (e.g. C/EBPα for mature B cells reprogramming efficiency) (FIG. 6E and (Hanna et al., 2008)).

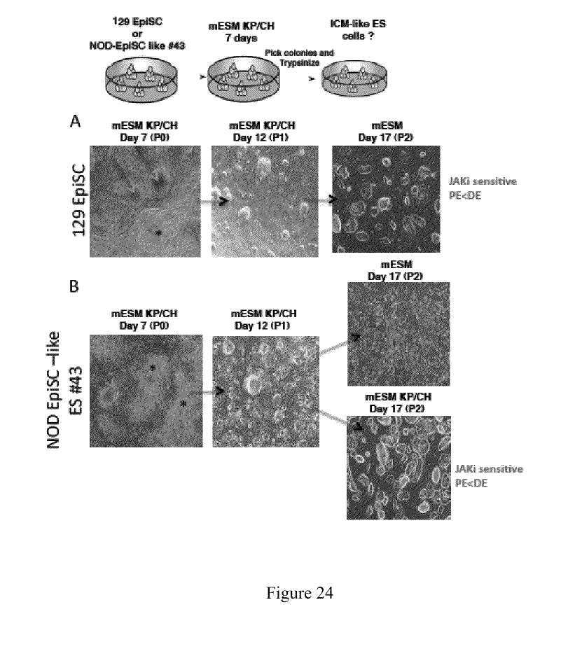

Finally, this work tested whether similar manipulations could convert the identity of NOD EpiSC-like cells to that of the ICM-like pluripotent state. NOD EpiSC-like ES cells were derived from germline competent NOD-ES#43 by withdrawing KP/CH and growing the cells in epiESM for over 8 passages (FIG. 6F). Subsequently, the cells were infected with TetO-Klf4 and grown in mESM and Dox. This treatment readily converted the EpiSC-like into ICM-like cells. However, the ICM-like pluripotent state on the NOD background remained stable only in the presence of Dox inducing the Klf4 transgene or, alternatively, by supplementing mESM with KP/CH (FIG. 6F). Identical results were obtained when KP/CH or PD/CH instead of Klf4 and c-Myc transgenes were used to reprogram NOD and 129 EpiSC cells to ICM like pluripotency (FIG. 24). In summary, expression of Klf4 or c-Myc converts the EpiSC-like state to ICM-like pluripotency, However, unlike 129 Epi-iPS cell lines, continuous presence of the same exogenous factors is required to stabilize the NOD Epi-iPS cells and to prevent reversion into the EpiSC-like state.

SUPPLEMENTARY TABLE 1
Summary for blastocysts injections and chimeras obtained
from different cell lines generated in this study.

| | 2N injections | | | |
|---|---|---|---|---|
| | Injected | | % | Germline |
| | blasto- | Born | Chimerism | Trans- |
| Cell line | cysts | chimeras | (coat color) | mission |
| NOD-iPS clone 1 | 20 | 1 | 10-30 | no |
| NOD-iPS clone 2 | 10 | 2 | 30-70 | no |
| NOD ES 33 (FUW-K) | 56 | 11 | 40-90 | no |
| NOD ES 26 (FUW-K) | 100 | 13 | 50-70 | yes |
| NOD ES 1M (FUW-M) | 45 | 5 | 20-60 | yes |
| NOD ES 85 KP | 70 | 17 | 40-90 | yes |
| NOD ES 43 KP/CH | 100 | 21 | 20-90 | yes |
| 129 Epi-iPS 1K | 60 | 10 | 30-90 | yes |
| 129 Epi-iPS 3M | 35 | 3 | 30-60 | no |
| NOD EpiSC-like | 70 | 0 | 0 | no |
| iPS #1 | | | | |
| NOD EpiSC-like | 75 | 0 | 0 | no |
| ES #1 | | | | |
| 129 TTF-iPS #3 | 30 | 4 | 20-70 | yes |
| (Derived in epiESM) | | | | |
| 129 ES #1 | 30 | 10 | 40-90 | yes |
| (Derived in epiESM) | | | | |
| NOD ES 75 (PD/CH) | 70 | 6 | 30-60 | yes |

SUPPLEMENTARY TABLE 2
Summary of NOD ES derivation in the presence of different small
molecules. Number of cell lines derived per method and efficiency
are indicated. Derivation of 129SvJae ES cells in the same method
without including inhibitors was used as a reference control.

| | | | # of ES lines |
|---|---|---|---|
| Mouse | Inhibitors and | | derived |
| strain | conditions used | Blastocysts | (% efficiency) |
| NOD/LtJ | mESM PD/CH | 60 | 3 |
| | | | (5%) |
| NOD/LtJ | mESM KP/CH | 54 | 11 |
| | | | (20%) |
| NOD/LtJ | mESM KP | 15 | 2 |
| | | | (13%) |
| 129SvJae | mESM | 12 | 10 |
| | | | (83%) |
| NOD/LtJ | epiESM | 30 | 4 |
| | | | (13%) |
| 129SvJae | epiESM | 20 | 3 |
| | | | (15%) |

Discussion

Stem cells characterized by different states of pluripotency and developmental potential have been derived under defined growth conditions. For example, growth of explanted mouse epiblasts in conditions containing bFGF and Activin, that are routinely used to isolate human ES cells, generates EpiSC cells that have a restricted ability to contribute to chimeric mice (Brons et al., 2007; Tesar et al., 2007; Thomson et al., 1998). In contrast, “mouse ES conditions” stabilize a pluripotent ES-like state with the potential to generate high-contribution chimeras in “permissive” mouse strains such as 129 (Ying et al., 2003). We found that identical culture conditions failed to induce ICM-like ES cells in the “non-permissive” NOD derived ICM explants. Stable transduction of the NOD ICM explants with c-Myc or Klf4 or addition of small molecules to the medium that replace the action of these factors during IFS derivation was sufficient to generate NOD ES cells that were indistinguishable from 129 ICM derived ES cells. The same stimuli were required to stabilize NOD fibroblast derived iPS cells, supporting the notion that in vitro stability of pluripotent state is dependent on genetic background rather than on the method used to derive the stem cells. However, the pluripotent state of NOD-ES cells was unstable despite the presence of Lif and could be maintained only through continuous expression of Klf4 or c-Myc or in the presence of inhibitors. Silencing of the transcription factors or removal of the inhibitors readily converted the cells to the EpiSC-like state. These cells resembled previously defined EpiSCs by multiple criteria including cellular morphology, signaling requirements and gene expression profiles (Tesar et al., 2007). We hypothesize that the NOD genome lacks or carries genetic determinants that cause instability of the ICM-like ES cell state in vitro unless supported by exogenous factors. It is also important to emphasize that our findings relate to differences in stem cells of different genetic background grown in culture rather than reflecting in vivo differences, as NOD embryos develop normally. Our defined conditions, either with inhibitors or over-expressing transcription factors, enable maintaining fully pluripotent NOD stem cells in vitro, and upon injection of the cells into host blastocysts, the in vivo environment probably substitutes for the requirement of exogenous factors and allows the cells to contribute to chimera formation.

### Metastable States of Pluripotency

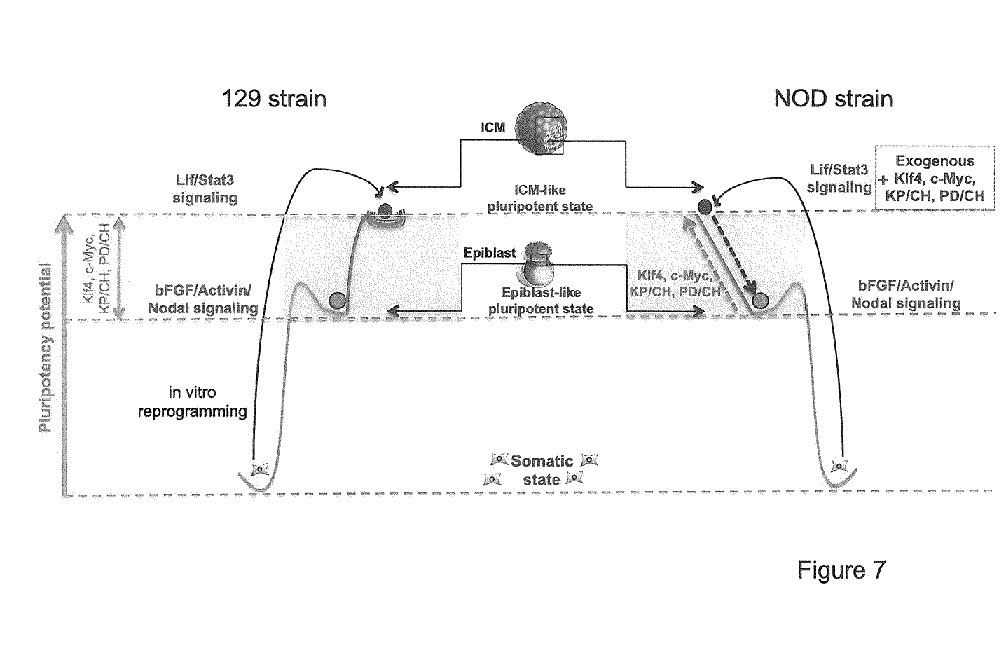

The term “metastability” has been previously used to describe transient changes within ICM-like ES cell populations resulting from oscillations in Nanog or Stella gene expression (Chambers et al., 2007; Hayashi et al., 2008). Here, we apply this term to describe the interconversion between two distinct pluripotent states in NOD and 129 mouse strains. Our results suggest that the ICM and EpiSC pluripotent states may be in a “metastable” equilibrium dictated by the genetic background where exogenous factors can convert one state into another. Thus, one may consider the two states of pluripotency, the ICM/ES cell-like state and the epiblast/EpiSC cell-like state, as two different levels of pluripotency (FIG. 7). Exogenous factors such as c-Myc and Klf4 in combination with Oct4 and Sox2 can induce the ICM-ES like state from somatic cells. However, the stability of the ES cell state is determined by the genetic background: while ICM-ES cells or iPS cells derived from a “permissive” genetic background such as 129 or C57BL/6 are stable once established in the presence of Lif, the ES cell like state of iPS cells or of ICM derived pluripotent cells of the “non-permissive” NOD background remains unstable with the maintenance of the pluripotent state depending on the continuous expression of the exogenous factors in addition to Lif/Stat3 signaling. Inactivation of the transcription factors or removal of the inhibitors causes the ES like NOD cells to assume an EpiSC-like state, characterized by reduced pluripotency. Inter-conversion between these states can be controlled by the absence or presence of the same factors (FIG. 7).

Several lines of evidence support the notion that the conversions between the different pluripotency states are due to cells being inefficiently induced to successfully convert from one state to another, rather than due to selection for rare pre-existing cells constantly present in heterogeneous stem cell populations. First, evidence for direct reprogramming of EpiSCs into iPS cells is supported by the observations that EpiSC cells do not convert spontaneously into ES like cells and that all derived Epi-iPS cell lines carried integrated viral transgenes (FIG. 24 and (Guo et al., 2009)). Second, the EpiSC to ES cell conversion requires multiple passages in defined media and continuous transgene induction, which is similar to generating iPS cells from somatic cells (FIG. 6, (Jaenisch and Young, 2008)). Third, it is unlikely that NOD ES cultures carry already rare EpiSC-like cells since the NOD iPS or ES lines were passaged routinely by trypsinization, which does not allow propagation of the EpiSC cells. Finally, the NOD EpiSC-like iPS cell line carried an identical Sox2 integration as its parental Dox dependent NOD iPS line indicating a clonal relation (FIG. 5B). An important question remains why only a small fraction of the NOD ES cells convert into an EpiSC state. One possibility is that after removal of the exogenous stimuli, the EpiSC state becomes one of several epigenetic states that can be acquired by the NOD ES cells upon differentiation.

### Pluripotency in NOD Strain as a Paradigm for Other “Non-Permissive” Species

It has been established that human and rhesus macaque ES cells resemble the EpiSC rather than the ICM pluripotent state of mouse cells suggesting that ICM-like pluripotent cells might have not been isolated from several species (Brons et al., 2007; Byrne et al., 2007; Lovell-Badge, 2007; Tesar et al., 2007; Thomson et al., 1998). When cultured under standard ES cell growth conditions, the pluripotent state of NOD stem cells isolated from explanted blastocysts or from somatic cells by in vitro reprogramming was the EpiSC-like state. The ICM-ES like state in NOD cells could only be stabilized when exogenous factors such as Klf4 or c-Myc were added. Thus, it is possible that “non-permissive” species such as human, that have yielded only EpiSC-like pluripotent cells, require specific exogenous factors to maintain the ICM-like pluripotent state. Consistent with this notion is that ICM-like iPS cells could be generated from rat fibroblasts under identical culture conditions to those used for the isolation of NOD iPS cells. We propose herein that the conditions that were successful for the isolation of NOD ICM-like ES cells could be used to maintain ICM-like ES from other “non-permissive” species. It will also be of great interest to define the genetic determinants that affect the in vitro stability of pluripotent states from different genetic backgrounds. Moreover, uncovering how Klf4, c-Myc, Wnt and MAPK pathways might converge and cross-talk in the reprogramming process, and whether they play a similar role in fibroblasts to iPS and EpiSC to iPS conversion, is a fundamental question relevant to understanding the mechanisms of reprogramming (Markoulaki et al., 2009; Marson et al., 2008; Silva et al., 2008a; Ying et al., 2008).

### Generation of Germline Competent NOD ES Cells

The inability to derive germline competent embryonic stem cells on the NOD background has posed limitations in generating genetically engineered NOD mice (Bach and Mathis, 1997). The NOD strain has been instrumental for the studying of disease progression and pathology of Insulin Dependent Diabetes Mellitus (IDDM). NOD mice spontaneously develop a form of diabetes that closely resembles human IDDM as a result of an autoimmune process directed against the pancreatic beta cells. IDDM is a polygenic disease with multiple parameters influencing susceptibility disease progression. Until now, the only available approach to generate an NOD knock-out involves crossing of NOD mice with a non-NOD strain carrying the desired allele and subsequently back-crossing to NOD mice for at least 15 generations to ensure re-establishment of the original NOD inbred genetic background harboring all IDDM susceptibility loci. Thus, the generation of germline competent NOD ES cells circumvents an obstacle posed on modeling IDDM in mice, and would establish an efficient platform for achieving direct gene targeting on the NOD background.

Supplementary Discussion

The role of reprogramming factors, particularly Klf4 and c-Myc is a subject of ongoing interest in the reprogramming field (Jaenisch and Young, 2008). Our results indeed might suggest a partially redundant role for c-Myc and Klf4 in reprogramming. This is in fact consistent with our previous work describing experiments on a transgenic mouse library with defined combination of reprogramming factors, where higher levels of Klf4 but not Sox2 or Oct4, were required to derive iPS lines from no c-Myc MEF lines (Markoulaki et al., 2009). Klf4 has been shown to occupy c-Myc promoter in ES cells, thus though the molecular pathways in which these two factors are involved are different, there is a potential crosstalk between the two (Kim et al., 2008). Uncovering how Klf4, c-Myc, Wnt and MAPK pathways might converge in the reprogramming process, and whether they play a similar role in fibroblasts to iPS and EpiSC to iPS conversion, is a fundamental question relevant to understanding the mechanisms of reprogramming (Jaenisch and Young, 2008; Silva et al., 2008; Silva and Smith, 2008; Takahashi et al., 2007; Ying et al., 2008).

Our defined conditions, either with inhibitors or over-expressing transcription factors, enable maintaining fully pluripotent NOD stem cells in vitro, and upon injection of the cells into host blastocysts, the in vivo environment probably substitutes for the requirement of exogenous factors and allows the cells to remain pluripotent and subsequently develop normally and contribute to chimera formation.

Expectedly, tumor formation was observed in some of the c-Myc transgenic NOD ES line derive chimeras and offspring (data not shown), probably as a result of the ectopic expression of the c-Myc oncogene (Markoulaki et al., 2009; Okita et al., 2007).

Supplemental Experimental Procedures

### Cell Culture and Viral Infections

mESM conditions refer to culturing the cells on irradiated MEFs in DME containing 15% FCS, leukemia inhibiting factor (Lif), 1 mM glutamine (Invitrogen), 1% nonessential amino acids (Invitrogen), 0.1 mM-mercaptoethanol (Sigma)). These cells (ICM-like) were passaged every third day as a singe cell suspension using 0.25% trypsin/EDTA. EpiSC and EpiSC-like cells were maintained in epiESM conditions which include maintaining the cells on mitomycin C inactivated mouse embryonic fibroblast (MEF) feeder layers in epiESM [DMEM/F12 (Invitrogen) supplemented with 15% FBS (Hyclone), 5% Knockout replacement serum (KSR—Invitrogen), 1 mM glutamine, 1% nonessential amino acids and 4 ng/ml human FGF2 (bFGF) (R&D systems)]. Cultures were passaged every 5 to 7 days either manually or enzymatically with collagenase type IV (Invitrogen; 1.5 mg/ml). The density of feeder cells was crucial to maintaining the EpiSC- and EpiSC-like cells in an undifferentiated state (density of 4*10̂4 cells per cm²). Lentiviral preparation and infection with Doxycycline inducible lentiviruses encoding Oct4, Klf4, c-Myc and Sox2 cDNA driven by the TetO/CMV promoter or constitutive lentiviruses driven by ubiquitin promoter, were done as previously described (Hanna et al., 2008; Hanna et al., 2007).

### Small Molecule Compounds

ALK inhibitor (ALKi-SB431542 Stemgent technologies) (20 μM final concentration); JAK inhibitor I (Calbiochem 420099—0.6 μM). Kenpaullone (KP) (Sigma—5μM), PD184352 (PD) (Pfizer—0.8 μM); CHIR99021 (Stemgent—3 μM).

Animals and ES derivation

NOD/ShiLtJ were obtained from the Jackson laboratory and bred in specific-pathogen-free animal facility. Control MEFs were made from 129SvJae mice. Non-NOD ES lines derived used in this study as controls were derived from matings between 129SvJae or B₆D₂F₁ mice. To derive ES lines, ICM explants were derived from day 3.5 blastocysts following procedures previously described (Markoulaki et al., 2008). Where applicable, ES derivation medium was also supplemented with the indicated compounds (Table S2). Dissociation of the outgrowths by treatment with Trypsin was performed on day 5 after plating and the cells were further cultured in mESC derivation medium until colonies appeared (typically after 5-7 days). From then on, established ES lines were cultured in mESM condition supplemented with the indicated compounds. NOD EpiSC-like ES lines were derived by similarly plating blastocysts in EpiSC-derivation medium (For 100 ml, we added 15 ml FBS (Hyclone), 5 ml KSR, 5 μl Lif (1×10̂7 U ESGRO/ml; Chemicon), 100 μl Mek1 inhibitor (PD98059; Cell Signaling Technology), hFGF2 (12 g/ml), 1 ml non-essential amino acids, 1 ml glutamine solution, and 1 ml pen/strep solution into DMEM/F12). ICM outgrowths were manually passaged after 5-7 days and were stably maintained in epiESM conditions. 129 EpiSC cell line used was previously described (Tesar et al., 2007), and were obtained from day E5.5 129SvEv (Taconic) embryos and propagated in epiESM conditions.

### Luciferase Reporter Assay

Constructs encoding the two previously characterized Oct4 enhancer (the Oct3/4 DE- and Oct3/4 PE-SV40-Luc constructs) cloned into the pGL3-Promoter Vector (Promega) were used to determine regulation pattern of Oct4 expression (Tesar et al., 2007). Constructs were transfected into 0.25-0.5*10̂6 cells using the Amaxa Nucleofection kit or Biorad along with the pRL-TK vector for normalization. Assays were performed 24-48 hours later using the Dual-Glo Luciferase Assay System (Promega). The basal activity of the empty luciferase vector was set as 1.0.

### Gene Array Expression Analysis and RT-PCR Analysis

RNA was isolated from MEF depleted mouse ES cells or from mechanically separated EpiSC and EpiSC-like cells lines using the RNeasy Mini Kit (Qiagen). Sample processing is described in supplementary Methods section. Heatmap and trees were visualized by Java Treeview. Microarray data are available at the NCBI Gene Expression Omnibus database (GSE15603). For RT-PCR analysis, 1 microgram of DNase I-treated RNA was reverse transcribed using a First Strand Synthesis kit (Invitrogen) and ultimately resuspended in 100 ul of water. Quantitative PCR analysis was performed in triplicate using 1/50 of the reverse transcription reaction in an ABI Prism 7000 (Applied Biosystems).

### REFERENCES

Bach, J. F. and Mathis, D. (1997). The NOD mouse. Res Immunol 148, 285-286.
Boyer, L. A., Lee, T. I., Cole, M. F., Johnstone, S. E., Levine, S. S., Zucker, J. P., Guenther, M. G., Kumar, R. M., Murray, H. L., Jenner, R. G., et al. (2005). Core transcriptional regulatory circuitry in human embryonic stem cells. Cell 122, 947-956.
Brons, L G., Smithers, L. E., Trotter, M. W., Rugg-Gunn, P., Sun, B., Chuva de Sousa Lopes, S. M., Howlett, S. K., Clarkson, A., Ahrlund-Richter, L., Pedersen, R. A., et al. (2007). Derivation of pluripotent epiblast stem cells from mammalian embryos. Nature 448, 191-195.
Buehr, M., Meek, S., Blair, K., Yang, J., Ure, J., Silva, J., McLay, R., Hall, J., Ying, Q. L., and Smith, A. (2008). Capture of authentic embryonic stem cells from rat blastocysts. Cell 135, 1287-1298.
Byrne, J. A., Pedersen, D. A., Clepper, L. L., Nelson, M., Sanger, W. G., Gokhale, S., Wolf, D. P., and Mitalipov, S. M. (2007). Producing primate embryonic stem cells by somatic cell nuclear transfer. Nature 450, 497-502.
Chambers, I., Silva, J., Colby, D., Nichols, J., Nijmeijer, B., Robertson, M., Vrana, J., Jones, K., Grotewold, L., and Smith, A. (2007). Nanog safeguards pluripotency and mediates germline development. Nature 450, 1230-1234.
Guo, G., Yang, J., Nichols, J., Hall, J. S., Eyres, I., Mansfield, and Smith, A. (2009). Klf4 reverts developmentally programmed restriction of ground state pluripotency. Development.
Hanna, J., Markoulaki, S., Schorderet, P., Carey, B. W., Beard, C., Wernig, M., Creyghton, M. P., Steine, E. J., Cassady, J. P., Foreman, R., et al. (2008). Direct reprogramming of terminally differentiated mature B lymphocytes to pluripotency. Cell 133, 250-264.
Hanna, J., Wernig, M., Markoulaki, S., Sun, C. W., Meissner, A., Cassady, J. P., Beard, C., Brambrink, T., Wu, L. C., Townes, T. M., et al. (2007). Treatment of sickle cell anemia mouse model with iPS cells generated from autologous skin. Science 318, 1920-1923.
Hatano, S. Y., Tada, M., Kimura, H., Yamaguchi, S., Kono, T., Nakano, T., Suemori, H., Nakatsuji, N., and Tada, T. (2005). Pluripotential competence of cells associated with Nanog activity. Mech Dev 122, 67-79.
Hayashi, K., Lopes, S. M., Tang, F., and Surani, M. A. (2008). Dynamic equilibrium and heterogeneity of mouse pluripotent stem cells with distinct functional and epigenetic states. Cell Stem Cell 3, 391-401.
Huangfu, D., Osafune, K., Maehr, R., Guo, W., Eijkelenboom, A., Chen, S., Muhlestein, W., and Melton, D. A. (2008). Induction of pluripotent stem cells from primary human fibroblasts with only Oct4 and Sox2. Nat Biotechnol 26, 1269-1275.
Jaenisch, R., and Young, R. (2008). Stem cells, the molecular circuitry of pluripotency and nuclear reprogramming. Cell 132, 567-582.
Jahner, D., Stuhlmann, H., Stewart, C. L., Harbers, K., Lohler, J., Simon, I., and Jaenisch, R. (1982). De novo methylation and expression of retroviral genomes during mouse embryogenesis. Nature 298, 623-628.
Li, P., Tong, C., Mehrian-Shai, R., Jia, L., Wu, N., Yan, Y., Maxson, R. E., Schulze, E. N., Song, H., Hsieh, C. L., et al. (2008). Germline competent embryonic stem cells derived from rat blastocysts. Cell 135, 1299-1310.
Li, W., Wei, W., Zhu, S., Zhu, J., Shi, Y., Lin, T., Hao, E., Hayek, A., Deng, H., and Ding, S. (2009). Generation of rat and human induced pluripotent stem cells by combining genetic reprogramming and chemical inhibitors. Cell Stem Cell 4, 16-19.
Liao, J., Cui, C., Chen, S., Ren, J., Chen, J., Gao, Y., Li, H., Jia, N., Cheng, L., Xiao, H., et al. (2009). Generation of induced pluripotent stem cell lines from adult rat cells. Cell Stem Cell 4, 11-15.
Lovell-Badge, R. (2007). Many ways to pluripotency. Nat Biotechnol 25, 1114-1116.
Lyssiotis, C A., Foreman, R., Staerk, J., Garcia, M., Mathur, D., Maroulaki, S., Hanna, J., et al. (2009). Reprogramming of murine fibroblasts to iPS cells: chemical complementation of Klf4. (Submitted).
Markoulaki, S., Hanna, J., Beard, C., Carey, B. W., Cheng, A. W., Lengner, C. J., Dausman, Fu, D., Gao, Q., Wu, S., et al. (2009). Transgenic mice with defined combinations of drug-inducible reprogramming factors. Nat Biotechnol 27, 169-171.
Markoulaki, S., Meissner, A., and Jaenisch, R. (2008). Somatic cell nuclear transfer and derivation of embryonic stem cells in the mouse. Methods 45, 101-114.
Marson, A., Foreman, R., Chevalier, B., Bilodeau, S., Kahn, M., Young, R. A., and Jaenisch, R. (2008). Wnt signaling promotes reprogramming of somatic cells to pluripotency. Cell Stem Cell 3, 132-135.
Okita, K., Ichisaka, T., and Yamanaka, S. (2007). Generation of germline-competent induced pluripotent stem cells. Nature 448, 313-317.
Rossant, J. (2008). Stem cells and early lineage development. Cell 132, 527-531.
Shi, Y., Desponts, C., Do, J. T., Hahm, H. S., Scholer, H. R., and Ding, S. (2008). Induction of pluripotent stem cells from mouse embryonic fibroblasts by Oct4 and Klf4 with small-molecule compounds. Cell Stem Cell 3, 568-574.
Silva, J., Barrandon, O., Nichols, J., Kawaguchi, J., Theunissen, T. W., and Smith, A. (2008a). Promotion of reprogramming to ground state pluripotency by signal inhibition. PLoS Biol 6, e253.
Silva, S. S., Rowntree, R. K., Mekhoubad, S., and Lee, J. T. (2008b). X-chromosome inactivation and epigenetic fluidity in human embryonic stem cells. Proc Natl Acad Sci USA 105, 4820-4825.
Takahashi, K., and Yamanaka, S. (2006). Induction of pluripotent stem cells from mouse embryonic and adult fibroblast cultures by defined factors. Cell 126, 663-676.
Tesar, P. J., Chenoweth, J. G., Brook, F. A., Davies, U., Evans, E. P., Mack, D. L., Gardner, R. L., and McKay, R. D. (2007). New cell lines from mouse epiblast share defining features with human embryonic stem cells. Nature 448, 196-199.
Thomson, J. A., Itskovitz-Eldor, J., Shapiro, S. S., Waknitz, M. A., Swiergiel, J. J., Marshall, V. S., and Jones, J. M. (1998). Embryonic stem cell lines derived from human blastocysts. Science 282, 1145-1147.
Wakayama, T., Perry, A. C., Zuccotti, M., Johnson, K. R., and Yanagimachi, R. (1998). Full-term development of mice from enucleated oocytes injected with cumulus cell nuclei. Nature 394, 369-374.
Wernig, M., Meissner, A., Foreman, R., Brambrink, T., Ku, M., Hochedlinger, K., Bernstein, B. E., and Jaenisch, R. (2007). In vitro reprogramming of fibroblasts into a pluripotent ES-cell-like state. Nature 448, 318-324.
Xu, R. H., Peck, R. M., Li, D. S., Feng, X., Ludwig, T., and Thomson, J. A. (2005). Basic FGF and suppression of BMP signaling sustain undifferentiated proliferation of human ES cells. Nat Methods 2, 185-190.
Ying, Q. L., Nichols, J., Chambers, I., and Smith, A. (2003). BMP induction of 1d proteins suppresses differentiation and sustains embryonic stem cell self-renewal in collaboration with STAT3. Cell 115, 281-292.
Ying, Q. L., Wray, J., Nichols, J., Batlie-Morera, L., Doble, B., Woodgett, J., Cohen, P., and Smith, A. (2008). The ground state of embryonic stem cell self-renewal. Nature 453, 519-523.

Supplementary Experimental Procedures

### Cell Culture

mESM conditions refer to culturing the cells on irradiated MEFs in DME containing 15% FCS, leukemia inhibiting factor (Lif), 1 mM glutamine (Invitrogen), 1% nonessential amino acids (Invitrogen), 0.1 mM-mercaptoethanol (Sigma)). These cells (ICM-like) were passaged every third day as a singe cell suspension using 0.25% trypsin/EDTA. EpiSC and EpiSC-like cells were maintained in epiESM conditions which include maintaining the cells on mitomycin C (MMC)-inactivated mouse embryonic fibroblast (MEF) feeder layers in epiESM [DMEM/F12 (Invitrogen) supplemented with 15% FBS (Hyclone), 5% Knockout replacement serum (KSR—Invitrogen), 1 mM glutamine (Invitrogen), 1% nonessential amino acids (Invitrogen), 0.1 mM-mercaptoethanol (Sigma) and 4 ng/ml human FGF2 (bFGF) (R&D systems)]. Cultures were passaged every 5 to 7 days either manually or enzymatically with collagenase type IV (Invitrogen; 1.5 mg/ml). The density of feeder cells was crucial to maintaining the EpiSC- and EpiSC-like cells in an undifferentiated state (density of 4*10̂4 cells per cm²). Secondary drug inducible MEF cells carrying OSM transgenes were previously described (Markoulaki et al., 2009).

### Small Molecule Compounds

ALK inhibitor (ALKi—SB431542 Stemgent technologies) was maintained as a 20 mM stock solution in DMSO (vehicle) (final concentration used 20 μM); JAK inhibitor I (Calbiochem 420099) was maintained as a 10 mM stock in DMSO (final concentration used 0.6 μM). Kenpaullone (KP) (Sigma—final concentration 5 μM), PD184352 (PD) (Kind gift from Pfizer—0.8 μM); CHIR99021 (Stemgent—3 μM). Wnt3a conditioned media (Willert et al., 2003) was generated according to standard protocols (ATCC), and used in a 1:1 dilution with DMEM used in generation of mESM. Media with inhibitors was routinely replaced every 48 hours to sustain activity of compounds.

### Viral Infection

Lentiviral preparation and infection with Doxycycline inducible lentiviruses encoding Oct4, Klf4, c-Myc and Sox2 cDNA driven by the TetO/CMV promote or constitutive lentiviruses driven by ubiquitin promoter, were done as previously described (Hanna et al., 2008; Hanna et al., 2007). Retrovirus stocks were prepared by transient transfection of Phoenix-Eco cells using Fugene (Roche), and supernatants were harvested 48 hr later. pGEM lentiviral constructs encoding hNanog and Lin-28 were obtained from Addgene. Klf4 or c-Myc were cloned into the XbaI-NheI site and mOrange was cloned into the SphI site of a previously described FUW-TetO lentivirus lacking any other factors (Carey et al., 2009). EcoRI factor-2A-mOrange then were also cloned in Ubiquitin promoter driven FUW lentivirus.

### DNA Methylation Analysis

For the methylation status of Oct4 and Nanog promoters, bisulphate sequencing analysis was performed as described previously (Wernig et al., 2007). A total of 6-10 clones from each sample were sequenced in both directions.

### Animals and ES Derivation

NOD/ShiLtJ (NOD) were obtained from the Jackson laboratory and bred in the specific-pathogen-free facilities at the whitehead institute. Control MEFs were made from 129SvJae mice. Non-NOD ES lines derived in this study as controls were derived from mating between 129SvJae or B₆D₂F₁ mice. V6.5 mouse ES line (C57B/6×1219SvJae F1) was used as a control in the indicated experiments. To derive ES lines, ICM explants were derived from day 3.5 blastocysts following procedures previously described (Markoulaki et al., 2008). Briefly, zona-free blastocysts after Acid Tyrode's treatment were plated on irradiated MEFs in ES derivation medium (mESC-derivation medium: For 100 ml, we added 15 ml KSR (knock-out serum replacement, Gibco, Cat#1028-028), 5 μl Lif ESGRO (1×10̂7 U ESGRO/ml; Chemicon, Cat# ESG1106), 100 μl Mek1 inhibitor (PD98059; Cell Signaling Technology, Cat#9900; out of 50 mM stock solution), 1 ml non-essential amino acids, 1 ml glutamine solution, and 1 ml pen/strep solution into DMEM). Where applicable, ES derivation medium was also supplemented with the indicated compounds (Table S2). Dissociation of the outgrowths by treatment with Trypsin was performed on day 5 after plating and the cells were further cultured in mESC derivation medium until colonies appeared (typically after 5-7 days). From then on, established ES lines were cultured in mESM condition supplemented with the indicated compounds. NOD EpiSC-like ES lines were done by similarly plating blastocysts in EpiSC-derivation medium (For 100 ml, we added 15 ml FBS (Hyclone), 5 ml KSR, 5 μl Lif ESGRO (1×10̂7 U ESGRO/ml; Chemicon, Cat# ESG1106), 100 μl Mek1 inhibitor (PD98059; Cell Signaling Technology, Cat#9900; out of 50 mM stock solution), hFGF2 (12 g/ml), 1 ml non-essential amino acids, 1 ml glutamine solution, and 1 ml pen/strep solution into DMEM/F12). ICM outgrowths were manually passaged after 5-7 days and were stably maintained in EpiESM conditions. 129 EpiSC cell line used was previously described (Tesar et al., 2007), and were obtained from day E5.5 129SvEv (Taconic) developing embryos and propagated in epiESM conditions. Rat tail tip fibroblasts were derived from adult female Sprague-Dawley strain (Charles River).

### Generation of Chimeras and Teratoma Formation

Diploid B₆D₂F₂ blastocysts (94-98 h after hCG injection) were placed in a drop of M2 medium under mineral oil. A flat-tip microinjection pipette with an internal diameter of 16 μm was used for cell injection using a Piezo micromanipulator (Primetech, Japan). About 10 cells were injected into the blastoceol cavity. After injection, the embryos were cultured in KSOM medium and placed in a 37° C. humidified CO₂ chamber until transferred to recipient females. Ten to fifteen injected blastocysts were transferred to each uterine horn of 2.5 days post coitum pseudo-pregnant B₆D₂F₁ females. For teratoma generation, 2*10̂6 cells were injected subcutaneously into both flanks of recipient SCID mice, and tumors were harvested for sectioning 3-6 weeks after initial injection. EpiSC-like cells were collagenased and manually dissociated into small pieces and injected subcutaneously in to immuno deficient mice.

### Gene Array Expression Analysis

RNA was isolated from MEF depleted mouse ES line by preplating on gelatin or from mechanically separated EpiSC and EpiSC-like cells lines using the RNeasy Mini Kit (Qiagen). Cy3-dye labeled cRNA samples were prepared using Agilent's QuickAmp sample labeling kit. Input was 0.5 μg total RNA. Briefly, first and second strand cDNA are generated using MMLV-RT enzyme and an oligo-dT based primer. In vitro transcription is performed using T7 RNA polymerase and either cyanine 3-CTP or cyanine 5-CTP, creating a direct incorporation of dye into the cRNA. Agilent (mouse 4×44k) expression arrays were hybridized according to our lab's method, which differs slightly from the Agilent standard hybridization protocol. The hybridization cocktail consisted of 1.65 ug cy3-dye labeled cRNA for each sample, Agilent hybridization blocking components, and fragmentation buffer. The hybridization cocktails were fragmented at 60° C. for 30 minutes, and then Agilent 2× hybridization buffer was added to the cocktail prior to application to the array. The arrays were then hybridized for 16 hours at 60° C. in an Agilent rotor oven set to maximum speed. The arrays were treated with Wash Buffer #1 (6×SSPE/0.005% n-laurylsarcosine) on a shaking platform at room temperature for 2 minutes, and then Wash Buffer #2 (0.06×SSPE) for 2 minutes at room temperature. The arrays were then dipped briefly in acetronitrile before a final 30 second wash in Agilent Wash 3 Stabilization and Drying Solution, in the hood using a stir plate and stir bar at room temperature. Arrays were scanned using an Agilent scanner and the data was extracted using Agilent's Feature Extraction software. Microarray data were processed and normalized between arrays using Limma package. Top 5000 differentially expressed probes were selected by (largest) cross-array standard deviations of the normalized expression values. Prior to clustering, for each selected probe, expression values across all arrays were centered and normalized such that their mean is 0 and the sum of squares is 1. Arrays and probes were clustered by hierarchical clustering using Cluster 3.0 (Pearson correlation, pairwise average-linkage). Heatmap and trees were visualized by Java Treeview. Microarray data are available at the NCBI Gene Expression Omnibus database under the series accession number GSE15603.

### Immunofluorescence Staining

Cells were fixed in 4% paraformaldehyde for 20 minutes at 25° C., washed 3 times with PBS and blocked for 15 min with 5% FBS in PBS containing 0.1% Triton-X. After incubation with primary antibodies against Oct4 (Santa Cruz), Nanog (polyclonal rabbit, Bethyl) and SSEA1 (monoclonal mouse, Developmental Studies Hybridoma Bank) for 1 h in 1% FBS in PBS containing 0.1% Triton-X, cells were washed 3 times with PBS and incubated with fluorophore-labeled appropriate secondary antibodies purchased from Jackson Immunoresearch. Specimens were analyzed on an Olympus Fluorescence microscope and images were acquired with a Zeiss Axiocam camera.

### RT-PCR Analysis

Three micrograms of total RNA was treated with DNase I to remove potential contamination of genomic DNA using a DNA Free RNA kit (Zymo Research, Orange, Calif.). One microgram of DNase I-treated RNA was reverse transcribed using a First Strand Synthesis kit (Invitrogen) and ultimately resuspended in 100 ul of water. Quantitative PCR analysis was performed in triplicate using 1/50 of the reverse transcription reaction in an ABI Prism 7000 (Applied Biosystems, Foster City, Calif.) with Platinum SYBR green qPCR SuperMix-UDG with ROX (Invitrogen). Primers used for amplification of transgene (viral) encoded transcripts were as follows: c-Myc: F, 5′-ACCTAACTCGAGGAGGAGCTGG-3′ (SEQ ID NO: 1) and R, 5′-TCCACATAGCGTAAAAGGAGC-3′ (SEQ ID NO: 2); Klf4: F, 5′-ACACTGTCTTCCCACGAGGG-3′ (SEQ ID NO: 3) and R, 5′-GGCATTAAAGCAGCGTATCCA-3′ (SEQ ID NO: 4); Sox2: F, 5′-CATTAACGGCACACTGCCC-3′ (SEQ ID NO: 5) and R, 5′-CGCCCGGAGTCTAGCTCTAA-3′ (SEQ ID NO: 6); Oct4: F, 5′-AGCCTGGCCTGTCTGTCACTC-3′ (SEQ ID NO: 7) and R, 5′-GGCATTAAAGCAGCGTATCCA-3′ (SEQ ID NO: 8). To ensure equal loading of cDNA into RT reactions, GAPDH mRNA was amplified using the following primers: F, 5′-TTCACCACCATGGAGAAGGC-3′ (SEQ ID NO: 9); and R, 5′-CCCTTTTGGCTCCACCCT-3′ (SEQ ID NO: 10). Primers used for endogenous expression of pluripotency genes were as follows: mSox2: F, 5′-GCAGGGAGTTCGCAAAAGTCT-3′ (SEQ ID NO: 11) and R, 5′-GGCATTAAAGCAGCGTATCCA-3′ (SEQ ID NO: 12); mOct4: F, 5′-ACATCGCCAATCAGCTTGG-3′ (SEQ ID NO: 13) and R, 5′-AGAACCATACTCGAACCACATCC-3′ (SEQ ID NO: 14); mNanog F, 5′ CCTCCAGCAGATGCAAGAACTC-3′ (SEQ ID NO: 15) and R, 5′-CTTCAACCACTGGTTTTTCTGCC-3′ (SEQ ID NO: 16); mRex1 F, 5′-TGTCCCCAAATACCACTGACC-3′ (SEQ ID NO: 17) and R, 5′-GGCACTGATCCGCAAACAC-3′ (SEQ ID NO: 18). mKlf4: F, 5′-TGCTTGGTGAGTTGTGGTCCT-3′ (SEQ ID NO: 19) and R, 5′-AGAACTTTGGCTCCCCCGT-3′ (SEQ ID NO: 20); c-Myc: F, 5′-ACATCGCCAATCAGCTTGG-3′ (SEQ ID NO: 21) and R, 5′-AGAACCATACTCGAACCACATCC-3′ (SEQ ID NO: 22). Data were extracted from the linear range of amplification. All graphs of qRT-PCR data shown represent samples of RNA that were DNase treated, reverse transcribed, and amplified in parallel to avoid variation inherent in these procedures. Semi-quantitative RT-PCR was performed using previously published primers (Buehr et al., 2008), Primers for detecting of FUW provirus encoding c-Myc or Klf4 were: (1) 5′ in ubiquitin promoter: GAGTCTTGAATGGAAGACGCTTGTGAGG (2) 3′ (SEQ ID NO: 23) detecting c-Myc: ACTGAGGGGTCAATGCACTCGG (3) 3′ (SEQ ID NO: 24) detecting Klf4: CCTGGTGGGTTAGCGAGTTGGA (SEQ ID NO: 25).

### Cell Cycle Analysis and Karyotyping

ES cells were trypsinized and subsequently MEF-depleted by pre-plating for 45 minutes. 1×10̂6 cells were resuspended in 0.5 ml PBS followed by the addition of 0.5 ml of 100% ice-cold ethanol to the cells in a drop-wise while vortexing. After incubation for 20 minutes on ice, cells were harvested by centrifugation (1000 rpm for 5-7 minutes) and ethanol was decanted. Finally, 0.5 ml of 7AAD-RNAse solution [(final concentrations 50 ug/ml 7AAD (BD pharmingen 7-AAD cat. 51-68981E+100 ug/ml RNAse Type I-A in PBS)] was added to the cells. After 30 minutes of incubation, the samples were analyzed by flow-cytometry by using BD-FACS Calibur. Cell line karyotyping testing and analysis was performed by Cell Line Genetics Inc. by analyzing 20 independent clone from each line examined.

### Reprogramming Efficiency of EpiSC and Hematopoietic Cells

Hematopoietic cells were obtained from bone marrow and spleen of recently described “Reprogrammable” transgenic mice carrying identical Dox inducible copies of the reprogramming factors Oct4, Sox2, Klf4 and c-Myc, Rosa26-MertTA and a Nanog-GFP knock-in reporter (Markoulaki et al., 2009). Unlike iPS-chimeras (Hanna et al., 2008), all cells in these “reprogrammable” mice carry the same set of transgenes, thus no additional labeling is identify transgenic cells. Hematopioetic cells were single cell sorted on gelatinized 96 well plates and grown in mESM Dox with or without the indicated additional cytokines. Mature B cells were sorted at 500 cells per well due to their very low reprogramming efficiency in the absence of C/EBPalpha, Reprogramming efficiency for C/EBPalpha-infected mature B cells was performed as previously described (Hanna et al., 2008). Efficiency for Nanog-GFP+ cells was determined at day 25 after Dox inductions. Where indicated in the figure mESM+Dox media was supplemented with some of the following cytokines: Flt-3L, SCF, IL-7 (10 ng/ml each, Peprotech), LPS (5 ng/ml Sigma). Two independent experiments were performed, in each between 960-960 wells were plated for each sample. Efficiency was calculated by dividing the individual Nanog-GFP+ clones by number of plated wells per samples. Hematopoietic populations were identified based on the following markers: Hematopoietic stem cell enriched populations HSC (Lin−/c-Kit+/Sca-1+) and common myeloid progenitor population CMP (Lin−/c-Kit+/Sca-1−/CD34+/FcγRintermediate), Spleen Mature B cells (IgM+IgD+), Bone marrow derived Pro-B cells (B220+CD25+), spleen monocytes (CD11b+ cells). Lineage (Lin) antibody cocktail included biotinylated antibodies for CD3, CD4, CD8, Mac-1. B220, Gr-1 and Ter 119). Antibody conjugates and matched isotype controls were obtained from BD Biosciences, R&D systems or eBioscience, and stainings were performed as previously described (Forsberg et al., 2006). FACS ARIA flow cytometer was used for sorting and analyses were performed using FACS Diva software. For 129 EpiSCs reprogramming efficiency, EpiSCs were infected with TetO-Klf-4-2A-morange or TetO-cMyc-2A-mOrange lentiviruses and after 3 days of Dox inductions mOrange+infected cells were single cell sorted in 96 well plates and number of Nanog+wells containing stable Epi-iPS cells mESM was determined at day 25 for all samples (Dox was removed at day 21 in all experiments).

### SUPPLEMENTARY REFERENCES

Buehr, M., Meek, S., Blair, K., Yang, J., Ure, J, Silva, J., McLay, R., Hall, J., Ying, Q. L., and Smith, A. (2008). Capture of authentic embryonic stem cells from rat blastocysts. Cell 135, 1287-1298.
Carey, B. W., Markoulaki, S., Hanna, J., Saha, K., Gao, Q., Mitalipova, M., and Jaenisch, R. (2009). Reprogramming of murine and human somatic cells using a single polycistronic vector. Proc Natl Acad Sci USA 106, 157-162.
Forsberg, E. C., Serwold, T., Kogan, S., Weissman, I. L., and Passegue, E. (2006). New evidence supporting megakaryocyte-erythrocyte potential of flk2/flt3+ multipotent hematopoietic progenitors. Cell 126, 415-426.
Hanna, J., Markoulaki, S., Schorderet, P., Carey, B. W., Beard, C., Wernig, M., Creyghton, M. P., Steine, E. J., Cassady, J. P., Foreman, R., et al. (2008). Direct reprogramming of terminally differentiated mature B lymphocytes to pluripotency. Cell 133, 250-264.
Hanna, J., Wernig, M., Markoulaki, S., Sun, C. W., Meissner, A., Cassady, J. P., Beard, C., Brambrink, T., Wu, L. C., Townes, T. M., et al. (2007). Treatment of sickle cell anemia mouse model with iPS cells generated from autologous skin. Science 318, 1920-1923.
Markoulaki, S., Hanna, J., Beard, C., Carey, B. W., Cheng, A. W., Lengner, Dausman, J. A., Fu, D., Gao, Q., Wu, S., et al. (2009). Transgenic mice with defined combinations of drug-inducible reprogramming factors. Nat Biotechnol 27, 169-171.
Markoulaki, S., Meissner, A., and Jaenisch, R. (2008). Somatic cell nuclear transfer and derivation of embryonic stem cells in the mouse. Methods 45, 101-114.
Wernig, M., Meissner, A., Foreman, R., Brambrink, T., Ku, M., Hochedlinger, K., Bernstein, B. E., and Jaenisch, R. (2007). In vitro reprogramming of fibroblasts into a pluripotent ES-cell-like state. Nature 448, 318-324.

### Example 2

### Human Embryonic Stem Cells with Biological and Epigenetic Characteristics Similar to Those of Mouse ESC's

Human and mouse embryonic stem cells (ESCs) are derived from blastocyst stage embryos but have very different biological properties, and molecular analyses suggest that the pluripotent state of human ESCs isolated so far corresponds to that of mouse derived epiblast stem cells (EpiSCs). This example describes rewiring the identity of conventional human ESCs into a more immature state that extensively shares defining features with pluripotent mouse ESCs. This was achieved by ectopic induction of Oct4, Klf4 and Klf2 factors combined with LIF and inhibitors of glycogen synthase kinase 3β (GSK3β) and mitogen-activated protein kinase (ERK1/2) pathway. Forskolin, a protein kinase A pathway agonist that can induce Klf4 and Klf2 expression, transiently substitutes for the requirement for ectopic transgene expression. In contrast to conventional human ESCs, these epigenetically converted cells have growth properties, an X chromosome activation state (XaXa), a gene expression profile, and signaling pathway dependence that are highly similar to those of mouse ESCs. Finally, the same growth conditions allow the derivation of human induced pluripotent stem (iPS) cells with similar properties as mouse iPS cells. The generation of “naïve” human ESCs will allow the molecular dissection of a previously undefined pluripotent state in humans, and may open up new opportunities for patient-specific, disease-relevant research. Reference numerals below refer to the listing of references immediately following this example.

Mouse embryonic stem cells (mESCs) are derived from the inner cell mass (ICM) of developing blastocysts (1) and can be maintained indefinitely in vitro in a pluripotent state, as they retain the capacity to contribute to all somatic cell lineages when injected into host embryos, including the germ line (2). Recently an additional type of pluripotent cells was derived from the post-implantation epiblast of murine embryos termed epiblast stem cells (mEpiSCs) (3, 4). mESCs and mEpiSCs are molecularly and epigenetically distinct and therefore represent discrete pluripotent states recently termed as naïve and primed pluripotent states, respectively (5). ICM-derived “naïve” pluripotent stem cells efficiently contribute to chimeric embryos, maintain both X chromosomes in an active state (XaXa) in female cells, and are highly refractory in their potential to differentiate into primordial germ cells in vitro (6). mEpiSCs, or “primed” pluripotent cells, can give rise to differentiated teratomas, but are highly inefficient in repopulating the ICM upon aggregation or injection into host blastocysts, have predominantly undergone X chromosome inactivation (XiXa) and are poised for differentiation into primordial germ cell (PGC) precursors in vitro upon exposure to bone morphogenic protein 4 (BMP4) (3, 4, 7). Naïve and primed pluripotent cells retain distinct gene expression programs in vitro that result in unique growth and molecular characteristics. Naïve mESCs can be cloned with high efficiency, grow as packed dome colonies and are stabilized by LIF/Stat3 and destabilized by bFGF and TGFβ/Activin/Nodal signaling. In contrast, primed mEpiSCs are characterized by a flattened morphology, intolerance to passaging as single cells and dependence on bFGF and Activin/Nodal signaling rather than LIF/Stat3. EpiSCs from 129 mouse strains can be reverted to naïve mESC-like cells upon exposure to LIF/Stat3 signaling and this conversion can be boosted by transient expression of pluripotency factors including Klf4, Klf2, Nanog or c-Myc (8-11).

The recent derivation of naïve pluripotent stem cells from non-obese diabetic (NOD) mouse strains and from rats, previously considered “non-permissive” for ESC derivation, has been achieved by culturing the cells in medium supplemented with small molecules or growth factors that alleviate inhibitory differentiation cues and/or reinforce key signaling pathways that stabilize the core transcriptional circuitry of naïve pluripotency (e.g. inhibition of glycogen synthase kinase 3β (GSK3β) and mitogen-activated protein kinase pathway (ERK1/2) in addition to LIF/Stat3 or exogenous constitutive expression of Klf4 or c-Myc) (5, 11-13). These conditions compensate for genetic determinants, which may be unique to each strain or species but preclude the in vitro propagation of naïve pluripotency. Notably, these genetic determinants do not interfere with the establishment and maintenance of the primed pluripotent state, as EpiSCs from both rat and NOD backgrounds are readily isolated (4, 11). NOD naïve pluripotent cells when isolated from pre-implantation blastocysts or generated through in vitro reprogramming are highly unstable and convert to a primed pluripotent state in vitro upon withdrawal of the exogenous supporting inhibitors or factors and addition of bFGF/Activin (11).

Although not identical, human ES cells (hESCs) share several defining features with primed mEpiSCs and are functionally and molecularly distinct from naive mouse ESCs (14). Both mEpiSCs and hESCs share a flattened morphology, intolerance to passaging as single cells, dependence on Activin/Nodal signaling (15), inactivation of the X chromosome in the vast majority of female cell lines isolated (16), and a propensity to differentiate into PGCs in response to BMP4 in vitro (17). The similarities between hESCs and mEpiSCs in addition to the aforementioned highly “metastable” naïve NOD pluripotent cells have underscored the possibility that the establishment and maintenance of the primed pluripotent state in human cells may reflect an inherent instability of naïve pluripotency that cannot be stabilized by the conventional culture conditions used for propagation of hESCs (11). These observations have provoked further questions relating to the nature of in vitro isolated hESCs and hiPSCs and the stability of pluripotent cells from different species (5, 11, 18).

Work described in this example sought to address some of these questions and define exogenous factors that stabilize novel human pluripotent states in vitro that share defining features with the naïve pluripotent cells of mice by molecular and functional criteria. These conditions allow the isolation of hiPSCs and epigenetic reversion of conventional hESCs toward a naïve pluripotent state. These findings provide the first evidence for a validated and previously undefined naïve state of pluripotency in humans.

Materials and Methods

### Culture and Differentiation of Human Pluripotent Cells

hiPSCs and the hESC lines BG01 (National Institutes of Health [NIH] code: BG01, BresaGen, Inc.) and WIBR3 hESC (28) were maintained in 20% pO2 conditions (unless indicated otherwise) and on mitomycin C (MMC)-inactivated mouse embryonic fibroblast (MEF) feeder layers in hESC medium (DMEM/F12 (Invitrogen) supplemented with 15% FBS (Hyclone), 5% KnockOut Serum Replacement (Invitrogen), 1 mM glutamine (Invitrogen), 1% nonessential amino acids (Invitrogen), 0.1 mM β-mercaptoethanol [Sigma], and 4 ng/ml bFGF (R&D Systems). Cultures were passaged every 5 to 7 days either manually or enzymatically with collagenase type IV (Invitrogen; 1.5 mg/ml). For EB-induced differentiation, naïve-hESC/hiPSC cell were trypsinized, separated from the MEF feeder cells by pre-plating on gelatin coated plates and cultured for 6-8 days in non-adherent suspension culture dishes (Corning) in DMEM supplemented with 15% FBS. Differentiation of into PGC precursors was performed as described previously (17). C1 hiPSCs were infected with a lentivirus harboring a VASA-EGFP reporter construct, selected with neomycin, subcloned and subsequently used for PGC differentiation protocol (17). For transfection of hiPSC and hESCs lines, cells were cultured in Rho Kinase (ROCK) inhibitor (Calbiochem; Y-27632) 24 hr prior to electroporation. Cells were harvested with 0.05% trypsin-EDTA solution (Invitrogen), and cells resuspended in PBS were either transfected with 75 μg DNA constructs (Gene Pulser Xcell System, Bio-Rad: 250 V, 500 μF, 0.4 cm cuvettes). Cells were subsequently plated on MEF feeder layers (DR4 MEFs for puromycin selection) in hESC medium supplemented with ROCK inhibitor for the first 24 hr. Notably, established naïve-hiPSCs/hESCs were not subjected to treatment with ROCKi before trypsinization or after genetic manipulation.

Naïve human pluripotent cells were grown in serum-free N2B27 based media. 500 ml of media was generated by including: 240 ml of DMEM/F12 (Invitrogen #11320), 240 ml of Neurobasal media (Invitrogen #21103), 5 ml of N2 supplement (Invitrogen #17502048), 10 ml B27 supplement (17504044), 10 μg of recombinant human LIF (Millipore, LIF1005), 1 mM glutamine (Invitrogen), 1% nonessential amino acids (Invitrogen), 0.1 mM β-mercaptoethanol (Sigma), pen/strep (Invitrogen), 5 mg/ml bovine serum albumin (Sigma) and small molecule inhibitors as indicated. Naïve-hESCs/hiPSCs were kept on mitomycin C (MMC)-inactivated mouse embryonic fibroblast (MEF) feeder cells, unless indicated otherwise, and where passage by single cell trypsinization every 5-10 days. Throughout the manuscript, indicated passage numbers of naïve-hiPSC/hESCs indicates number of passages counted after induction of the naïve state. The C1 secondary fibroblast cells (19), harbors Doxycycline (DOX) lentiviral vectors encoding Oct4, Sox2 and Klf4 reprogramming factors (45) and a constitutively active lentivirus encoding the reverse tetracycline transactivator (19), were grown in the presence of Dox in N2B27 media supplemented with different inhibitor combinations on gelatin coated plates, until initial PSC colonies were observed and subcloned. Mouse EpiSCs were derived and propagated as previously described (3,4). NOD ESC #43 line was used in this study (11) and expanded in N2B27 PD/CH/LIF, For teratoma formation and analysis, hESC cells were collected by collagenase treatment (1 mg/ml) and separated from feeder cells by subsequent washes with medium and sedimentation of hESC cell colonies. Naïve-hESCs and hiPSCs were harvested by trypsinization prior to injection. Cells were injected subcutaneously into SCID mice (Taconic, Hudson, N.Y.). Tumors generally developed within 4-6 weeks and animals were sacrificed before tumor size exceeded 1.5 cm in diameter. For directed neural differentiation, naïve human iPS cells were dissociated with Accutase (Invitrogen) for 15 minutes into a single cell suspension. MEFs were excluded by plating for one hour on gelatin at 37 C. The remaining pluripotent cells were plated on matrigel (BD) in mTESR1 (Stem Cell, Inc.) at 3.5×10̂4 cells per cm2, in 35 mm dishes. The cells were allowed to reach confluence in mTESR1 for 2 days, and shifted to KSR medium containing 10 uM SB431542 (Stemgent), and 500 ng/mL of Noggin (Stemgent). After 7 days of daily medium change, some cells were stained for Pax6 (covance rabbit anti-Pax6, 1:200) and Nestin (Chemicon, 1:200), followed by appropriate alexa-conjugated secondary antibodies (Invitrogen, 1:500). The large majority of cells expressed both markers of a neural progenitor fate. The remaining cells were adapted progressively to N2 medium, and finally passaged on day 10 to N2 medium containing 20 ng/mL EGF and 20 ng/mL bFGF to maintain the neural progenitor population. To generate neurons, these neural progenitors were plated at a density of 10̂4/cm̂2 in 35 mm dishes coated with matrigel, and fed daily with N2 medium without growth factors. After a week in culture, the differentiated cells were stained using a mouse anti-Tuj 1 antibody (Covance, 1:300), or a mouse anti-MAP2 (sigma, 1:200), followed by appropriate alexa-conjugated secondary antibodies (Invitrogen).

### Small Molecule Compounds and Cytokines

Small molecules and cytokines were purchased from Tocris, Calbiochem or Sigma, and were supplemented as indicated at the following final concentration: JAK inhibitor (JAKi, 6 μM), Kenpaullone (KP, 5 μM), PD0325901 (PD, 1 μM); CHIR99021 (CH, 304), Forskolin (FK, 10 μM), FGF4-Receptor inhibitors PD173074 (0.1 μM) and SU5401 (2 μM); TGFβ/ALK inhibitors A83-01 (1 μM), RepSox (1 μM), ALK inhibitor (ALKi—SB431542, 2 μM); AICAR (0.5 mM); BixO1294 (1 μM); BayK8644 (1 μM); recombinant human BMP4 (10 ng/ml), recombinant human IL-6 (10 ng/ml), recombinant human TGFβ (500 ng/ml), Media with inhibitors was routinely replaced every 48 hours to sustain activity of compounds.

### DNA Constructs

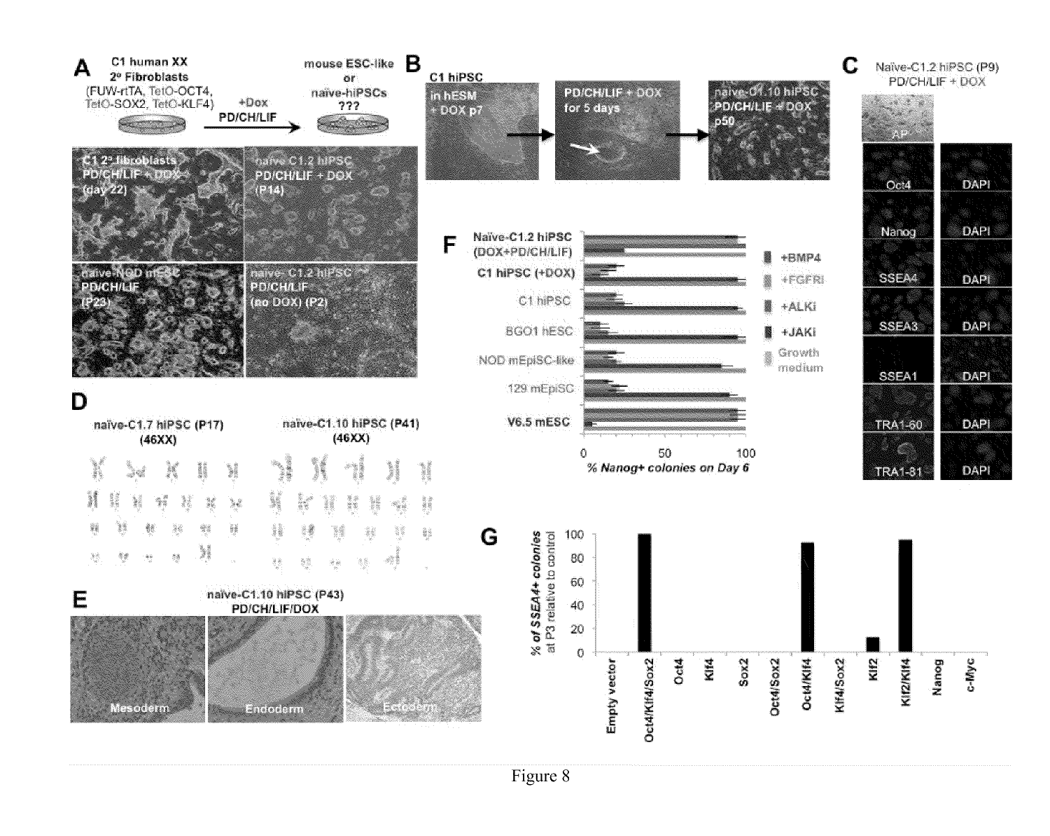

The pCAG-IresPuro or pCAG-flox-DsRed-IRES-Puro vectors (46) encoding the following inserts (which were cloned by either cohesive or blunt end ligations in XhoI-NotI sites): KLF2, Klf4, Oct4-2A-Klf4, Oct4, Nanog, mSox2, Stat3-C (A662C and N664C mutations) (31), Stat3-A662C/N664C (dominant negative-DN allele). PBS246-CAAGS-Oct4/Klf4/Sox2 and constitutively expressed lentivirus FUW-cMyc (11) were also used in FIG. 8G. Human Oct4 enhancer sequences (the Oct4DE- and Oct4PE-SV40-Luciferase (Luc) constructs) were cloned into the pGL3-Promoter Vector (Promega) with the following primers: 5′hOcCT4PE KpnI: GGTACCGGATACTCAGGCCAGGCCCAGAAA (SEQ ID NO: 26); 3′ hOCT4PE XhoI: CTCGAGTCCACAGACCTCTGGCACT (SEQ ID NO: 27); 5′ hOCT4DE KpnI: GGTACCCATTGAGTCCAAATCCTCTTTACTAGGTG (SEQ ID NO: 28); 3′ hOCT4DE XhoI: CTCGAGCTGAGGCTCATGCTGCTGG (SEQ ID NO: 29). Reporter constructs were used to determine regulation pattern of Oct4 expression and were electroporated into 0.5-3*10̂6 cells along with the pRL-TK vector for normalization. Assays were performed 48 hours later using the Dual-Glo Luciferase Assay System (Promega). The basal activity of the empty luciferase vector was set as 1.0.

### Immunocytochemistry and FACS Analysis

Cells were fixed in 4% paraformaldehyde in PBS and immuno-stained according to standard protocols using the following primary antibodies: SSEA4 and SSEA3 (Developmental Studies Hybridoma Bank); Tra-1-60 and Tra-1-81 (Millipore); SOX2 (R&D Systems); OCT3/4 (Santa Cruz Biotechnology); human NANOG (goat polyclonal, R&D Systems), mouse Nanog (polyclonal rabbit, Bethyl) and mouse SSEA1 (Developmental Studies Hybridoma Bank). Appropriate Molecular Probes Alexa Fluor dye-conjugated secondary antibodies (Molecular Probes, Invitrogen, Carlsbad, Calif.) were used. For MHC class I staining of human cells we used W6/32 mAb (a kind gift from O. Mandelboim, Hebrew University) directed against conformed MHC-I proteins. For mouse cells we used anti-mouse MHC Class I H-2 Kb and H-2 Kd (ebiosciences).

### RNA FISH

RNA FISH was carried out as previously described (28). Briefly, human pluripotent stem cells were harvested, MEF depleted and cytospun onto glass slides prior to fixation. cDNA probes were generated to XIST exon 1 (GenBank U80460: 61251-69449) and exon 6 (U80460: 75081-78658), labeled by nick translation (Roche, Indianapolis, Ind.) with Cy3-dUTP (Amersham) and Cot-1 DNA was labeled with fluoroscein-12-dUTP using the Prime-It Fluor Labeling kit (Stratagene, La Jolla, Calif.).

### Gene Expression Analysis

Microarray data are available at the NCBI Gene Expression Omnibus database under the series accession number GSEXXXX. Human Affymetrix HG-U133 array data were processed by Affymetrix R package and using MAS 5.0 algorithm (47). Probe sets were remapped to ensemb1 genes using custom CDFs from University of Michigan BrainArray site (http://brainarray.mbni.med.umich.edu/Brainarray/Database/CustomCDF/CDF_download.asp). Top 500 differentially expressed genes by standard deviation were selected to hierarchical cluster by Pearson correlation and average-linkage (gene-centered by median and gene-normalized) using Cluster 3.0 software and visualized in JavaTreeView. Expression values were Log transformed, gene-centered and normalized prior to clustering. Similar trees were observed when clustering all genes or top 500 or 5000 differentially expressed genes. Data presented in FIG. 12B were extracted from human array dataset that was processed as above [MAS5.0] but not using University of Michigan BrainArray CDF files because the mapping algorithm discards some of the genes of interest by their criteria. Probesets targeting the same genes were collapsed by median. Values from naïve-hESC lines were averaged. Values from other hESC cells were also averaged. Barcharts in FIG. 12B were plotted as ratio of these group means to the median of all cell lines.

Cross-species gene expression analysis was conducted on Human arrays described above and previously described mouse ESC and EpiSC gene expression dataset on Agilent 4×44k array platform (GSE15603; described in (11)). Mouse data was processed by with-array LOESS and across-array quantile normalization using limma R package (48). Probes targeting the same gene were collapsed by median. Human-mouse orthology was downloaded from Biomart (http://www.biomart.org/) and selecting only orthologous pairs with one2one orthology type with at least 70% reciprocal identities. Of these 11577 pairs of human-mouse genes, 9949 of them can be mapped to our expression data. The expression values from mouse and human were transformed separately into relative abundance (RA) values (49): for each gene, the RA value is the expression value divided by the sum of expression values within the same gene across samples in the same species. The resulting expression matrix was subjected to hierarchical clustering (Spearman correlation, average-linkage) using Python Cluster3 library and assessed by bootstrapping using pvclust R package (35).

### RT-PCR Analysis

Three micrograms of total RNA was treated with DNase I to remove potential contamination of genomic DNA using a DNA Free RNA kit (Zymo Research, Orange, Calif.). One microgram of DNase I-treated RNA was reverse transcribed using a First Strand Synthesis kit (Invitrogen) and ultimately resuspended in 100 ul of water. Quantitative PCR analysis was performed in triplicate using 1/50 of the reverse transcription reaction in an ABI Prism 7000 (Applied Biosystems, Foster City, Calif.) with Platinum SYBR green qPCR SuperMix-UDG with ROX (Invitrogen) (17, 19, 28, 39, 50, 51).

### DNA Methylation Analysis

Genomic DNA was collected from hESCs and hiPSCs following separation from feeder cells. DNA was proteinase K treated and extracted and 1 μg of DNA was subjected to conversion using the Qiagen EpiTect Bisulfite Kit. Promoter regions of OCT4, NANOG and XIST were amplified using previously described primers (19, 28, 50). PCR products were cloned using the pCR2.1-TOPO vector and sequenced using M13 forward primer.

Results

Stabilization of the Naïve Pluripotent State in Human Stem Cells

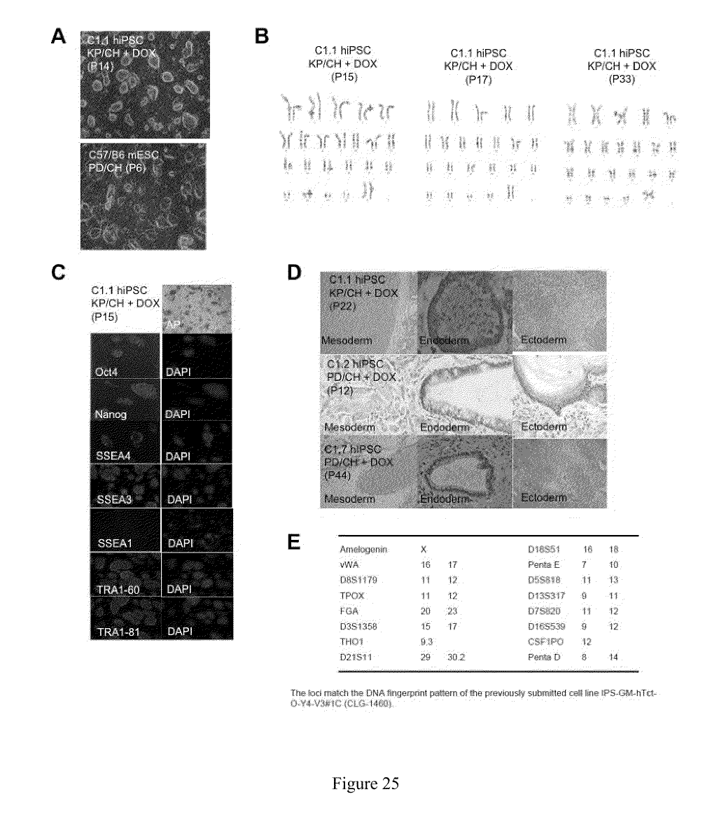

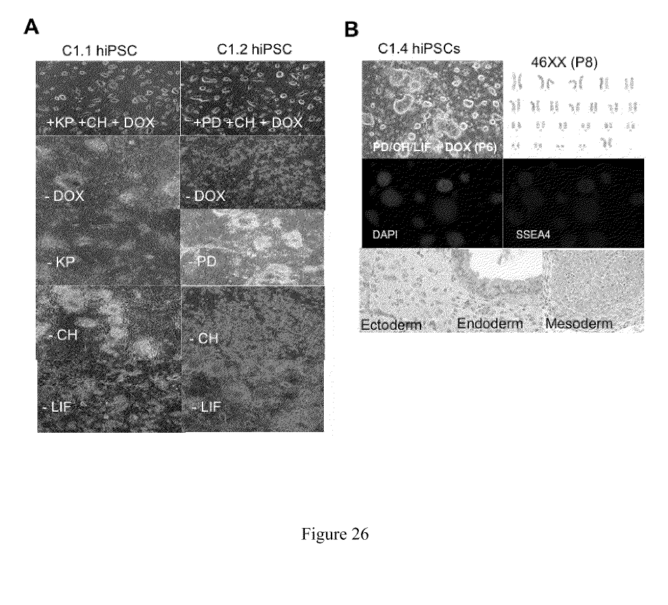

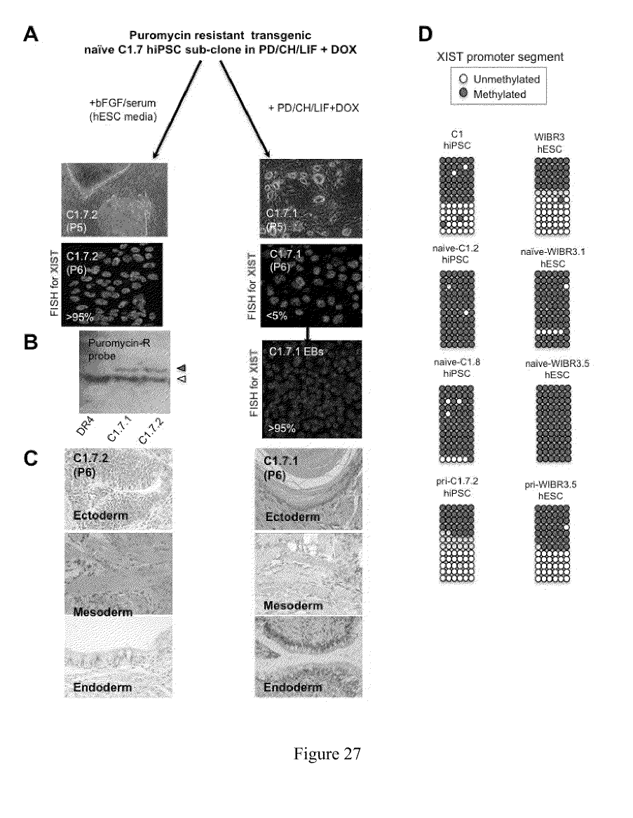

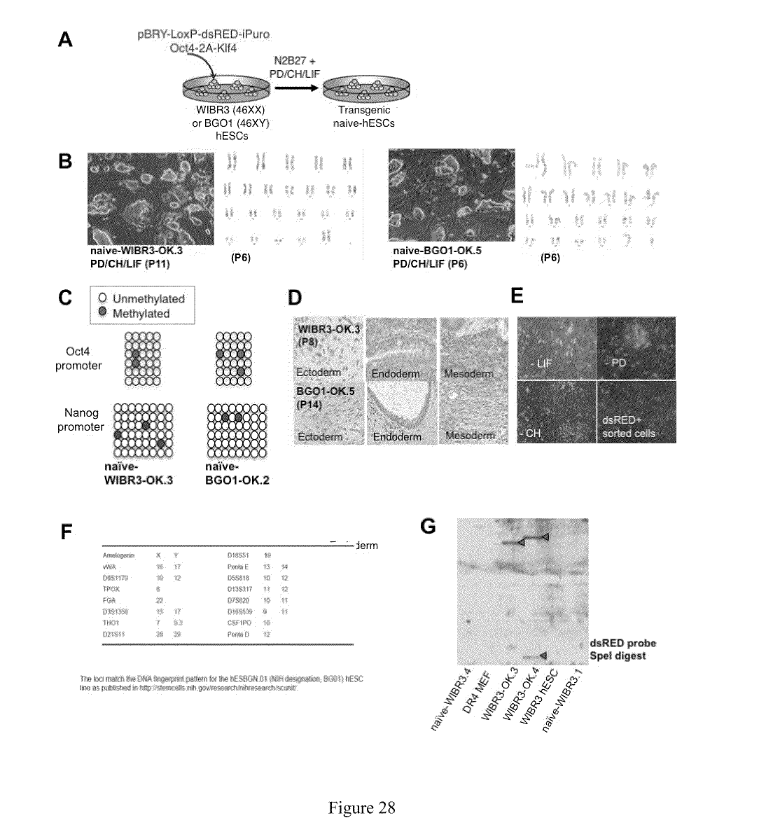

To test whether conditions devised previously to stabilize mouse NOD and rat ESCs (5, 11, 12) influence the properties of human pluripotent stem cells in vitro, the previously described C1 secondary human female fibroblasts (19) were used to derive induced pluripotent stem cells (hiPSCs) under various culture conditions (FIG. 8A). The C1 secondary fibroblast line harbors Doxycycline (DOX) lentiviral vectors encoding OCT4, SOX2 and KLF4 reprogramming factors and a constitutively active lentivirus encoding the reverse tetracycline transactivator. After 14-25 days of culture in serum-free N2B27 medium and a combination of the ERK1/2-cascade inhibitor PD0325901 (PD), the GSK3 inhibitor CHIR99021 (CH) and LIF (abbreviated as PD/CH/LIF), colonies with naïve mESC-like morphology appeared that could be maintained in media with DOX and PD/CH/LIF (FIGS. 8A and 25). Similar colonies were observed after transferring Dox independent C1-hiPSC lines grown in bFGF/serum-containing growth conditions into N2B27 PD/CH/LIF+Dox for 5-7 days (FIG. 8B). Individual C1 hiPSC-like clones were picked and further passaged with trypsin in the presence of DOX and inhibitors for over 50 passages. These lines were nearly indistinguishable morphologically from mESCs (FIG. 8A), expressed human pluripotency markers (FIGS. 8C and 26), remained karyotypically normal after extended passaging (FIG. 8D), displayed a pre-X inactivation epigenetic state (XaXa) as evidence by lack of XIST clouds (FIG. 27), generated differentiated teratomas (FIGS. 8E and 25), were insensitive to inhibition of bFGF and Activin/Nodal signaling or addition of BMP4 similarly to mouse ESCs (FIG. 8F), and could be stably grown in DOX+PD/CH/LIF in the absence of serum or feeder cells (FIG. 26). However unlike NOD murine iPSCs that could be maintained under these conditions independent of transgene induction by DOX (11), these C1 naïve-like hiPSCs rapidly differentiated upon withdrawal of DOX (FIGS. 8A, 9A and 26A and 26B). Constitutive transgene-mediated expression of Klf4 and Oct4 or Klf4 and Klf2 was required to propagate C1 hiPSC cells in the PD/CH/LIF condition independent of Dox (FIG. 8G). Similarly, BGO1 or WIBR3 hESC cells were stably transfected with Oct4 and Klf4 transgene and transferred into growth condition of N2B27 media with PD/CH/LIF (FIG. 28) and independent subclones were derived as shown by southern blot analysis for transgene integration (FIG. 28G). Stable pluripotent lines were established that expressed endogenous pluripotency genes (FIG. 28C), maintained a normal karyotype (FIG. 28B), generated mature differentiated teratomas (FIG. 28D) and required continuous expression of ectopic transgenes and PD/CH/LIF to remain stable in vitro (FIG. 28E). These results indicated that the human genetic background may be less “permissive” than that of the NOD mouse strain, and thus may require additional exogenous factors to allow stabilization of naïve pluripotent cells in vitro.

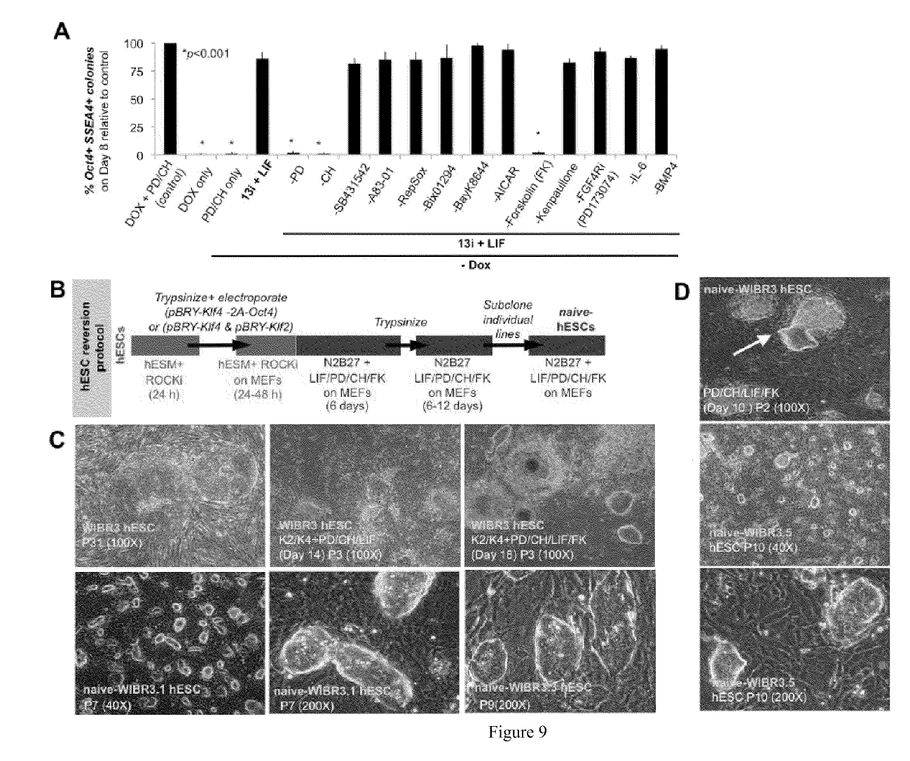

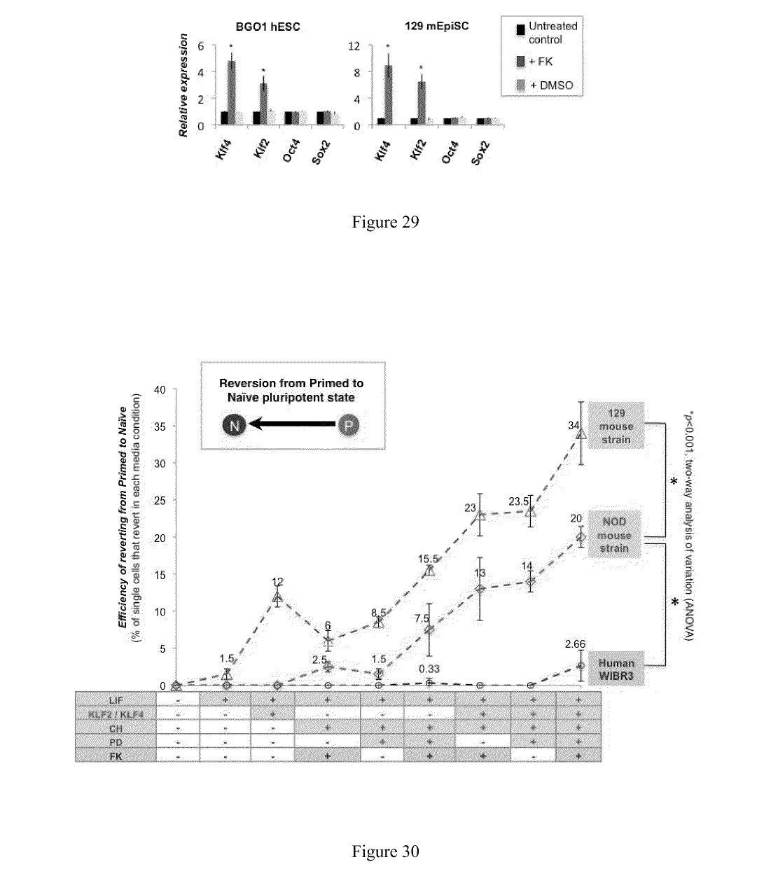

The DOX-dependent naïve C1.2 hiPSC line was next utilized to screen for other compounds and growth factors that could stabilize C1.2 hiPSCs upon DOX withdrawal in N2B27 PD/CH/LIF media (FIG. 9A). We included compounds that have previously been shown to support the pluripotency of naïve mESCs, such as inhibitors of TGFβ and FGF tyrosine kinase receptor signaling pathways, BMP4, IL-6 or molecules that substitute for Klf4/Klf2 in reprogramming or regulate their expression (Kenpaullone, BIX01294, BayK8644, Forskolin and AICAR) (20-23). As shown in FIG. 9A, the combined action of the perturbations (termed 13i+LIF conditions) allowed the stabilization of C1.2 hiPSCs in the absence of DOX. By removing one inhibitor at a time we identified PD, CH and Forskolin (FK) as crucial components in this cocktail of inhibitors (FIG. 9A). Forskolin, which was previously used for the propagation of human embryonic germ (EG) cells (24), activates the enzyme adenylate cyclase which increases the intracellular levels of cAMP and subsequently activates the protein kinase A (PKA) signaling pathway (25). Forskolin stabilizes C1 hiPSCs independently of DOX, at least partly through induction of Klf2 and Klf4 expression (FIG. 29).

Additional work tested whether the PD/CH/FK/LIF culture condition in combination with transient transgene induction could revert the established WIBR3 hESC line to a naive pluripotent state. WIBR3 hESCs, were transiently transfected with Oct4 and Klf4 or Klf4 and Klf2 and then grown in PD/CH/FK/LIF serum-free media. After 8-12 days dome-shaped colonies with packed round cell morphology, typical of naïve mESCs appeared (FIG. 9B-C and FIG. 30). Colonies were picked, trypsinized and passaged in PD/CH/FK/LIF conditions. Since these cell lines were morphologically similar to naïve mESCs we refer to the selected cells as naïve-hESCs (naive-WIBR3.1-3.4) and naïve-hiPSCs (naïve-C1.2-1.10). The WIBR3.5 naïve-hESC line was obtained without transient factor transfection (FIG. 9D).

Naïve hESCs and hiPSCs are Pluripotent

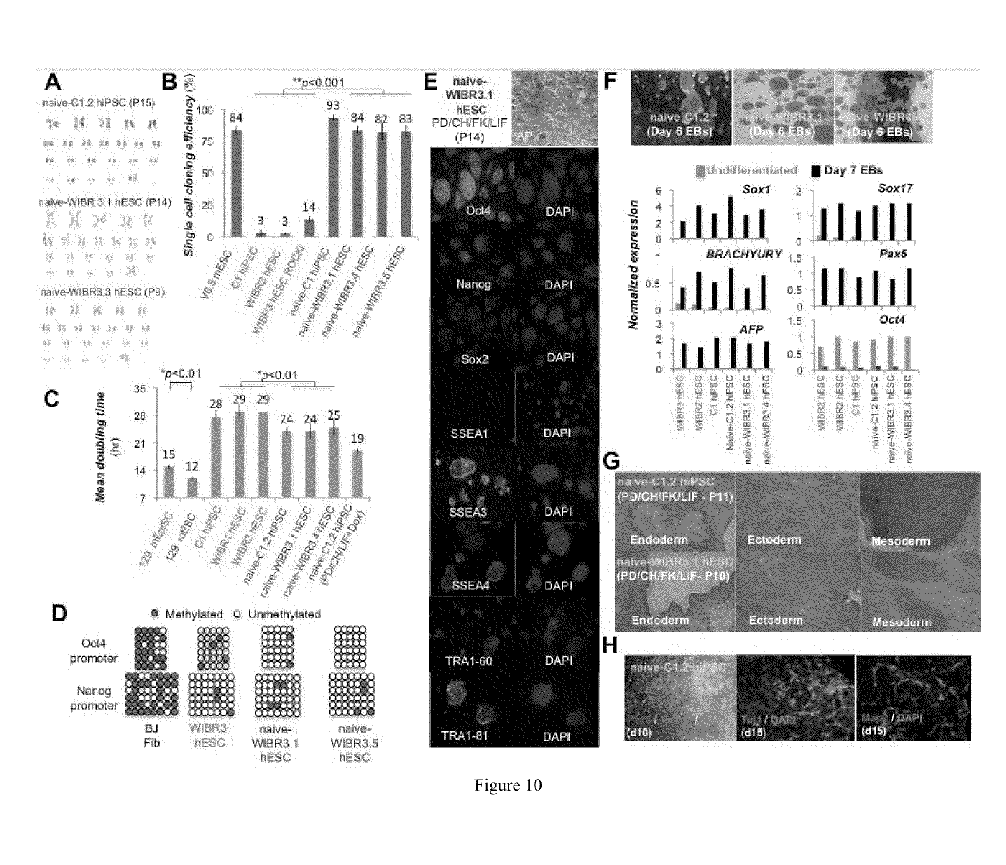

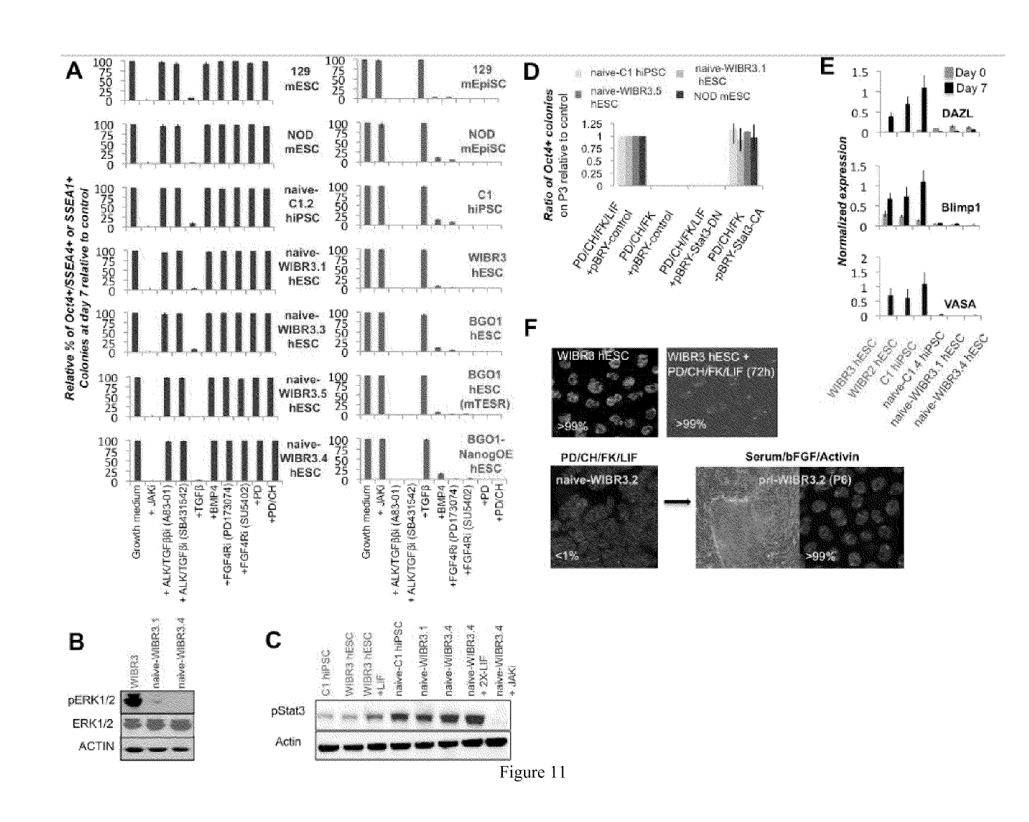

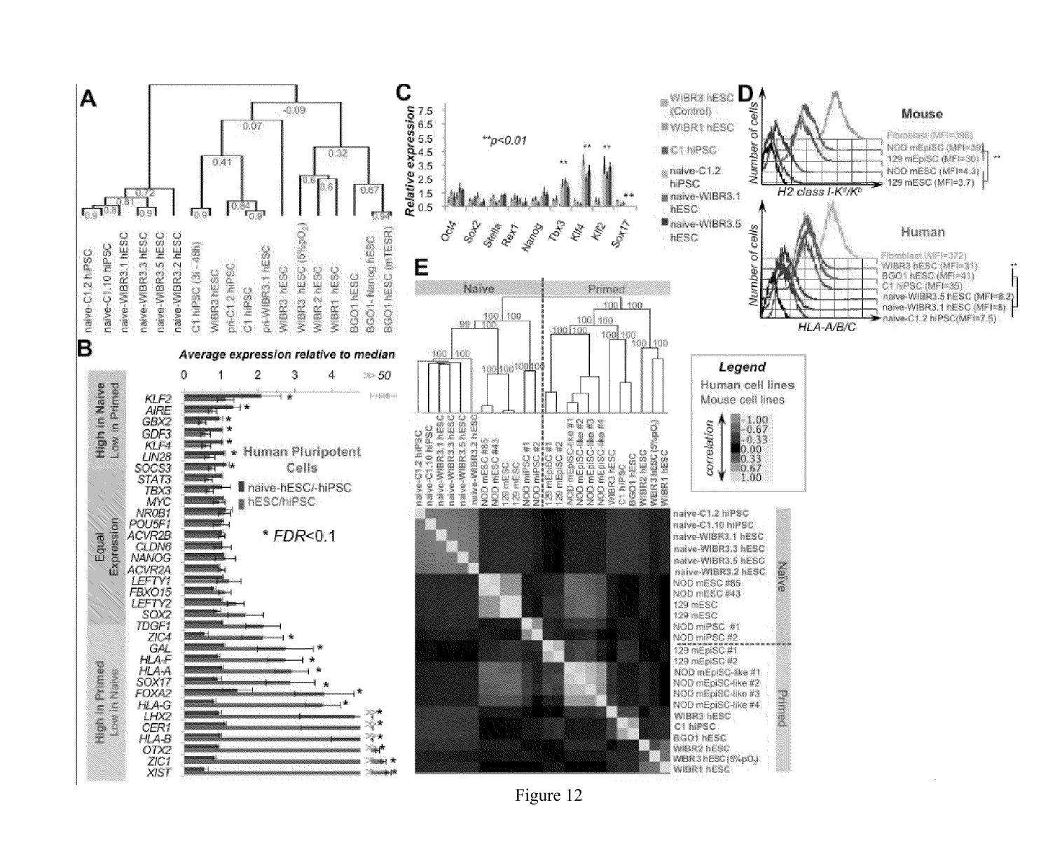

Naïve hESC and hiPSC lines were grown on feeder cells in PD/CH/FK/LIF, passaged using trypsin and demonstrated a normal karyotype (FIG. 10A). Naïve hESC and hiPSC cells displayed>85% single cell cloning efficiency after trypsinization, comparable to the high clonogenicity typical for mouse ESCs (FIG. 10B). In contrast, conventional hESCs and hiPSCs have a single cell cloning efficiency of less <1%, or ˜20% in the presence of Rho Kinase (ROCK) inhibitor (FIG. 10B). The average doubling time for naive-hESCs was slightly decreased by ˜20% (FIG. 10C). The Oct4 and Nanog promoters were hypomethylated in naive-hESCs in comparison to somatic fibroblasts (FIG. 10D) and the cells stained uniformly positive for pluripotency markers (FIG. 10E and FIG. 31). To determine the differentiation ability of naive-hESCs/hiPSCs in vitro, we used suspension culture to generate embryoid bodies (EBs). After 7 days in suspension RT-PCR confirmed that these EB-differentiated cells expressed markers of all three developmental lineages (FIG. 10F), The naive-hESC/hiPSCs were transplanted into immuno-deficient (SCID) mice and formed teratomas with somatic tissues representative of the three germ layers (FIG. 10G), indicating that naive-hESC/hiPSC are pluripotent. Naïve-hiPSC cells could be also directed to differentiate into neuronal cells in vitro (FIG. 10H). We indicate that the naïve pluripotent stem cells expressing the exogenous transgenes could be maintained to date for more than 50 passages in PD/CH/LIF (FIGS. 8 and 25) which is unlike the genetically unmodified forskolin-dependent naïve cells that could not be maintained for longer than 15-20 passages when they stopped proliferating and differentiated. This limited growth potential has been previously described for human EG cells propagated in the presence of forskolin (24) and may be due to toxicity associated with this compound or its inability to fully substitute for exogenous transgene expression (FIG. 8).

Naïve Human Pluripotent Cells Share Signaling and Functional Features with Mouse ESCs

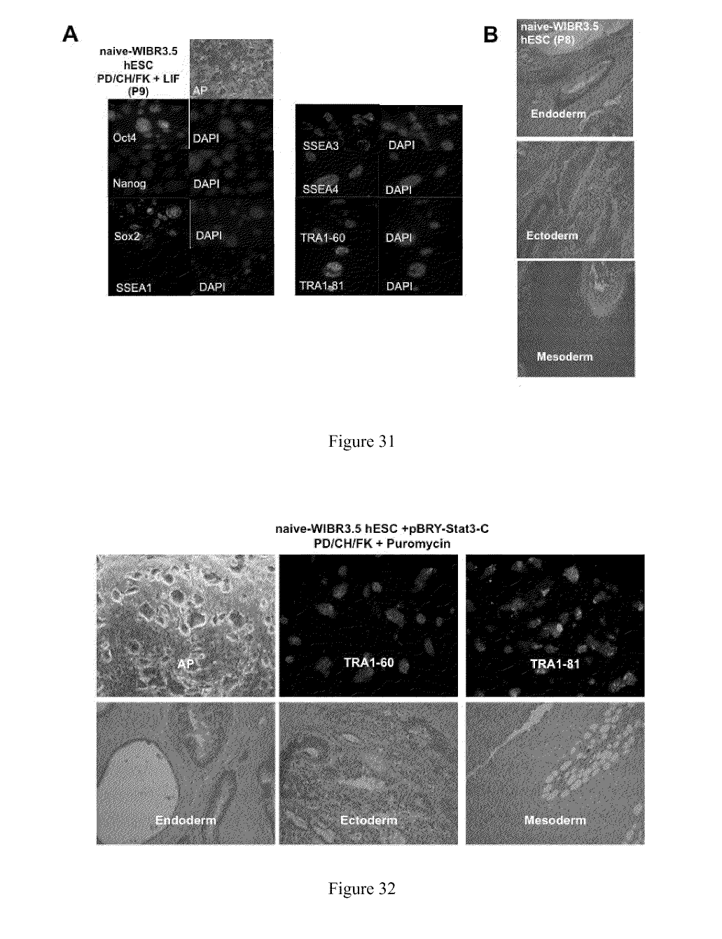

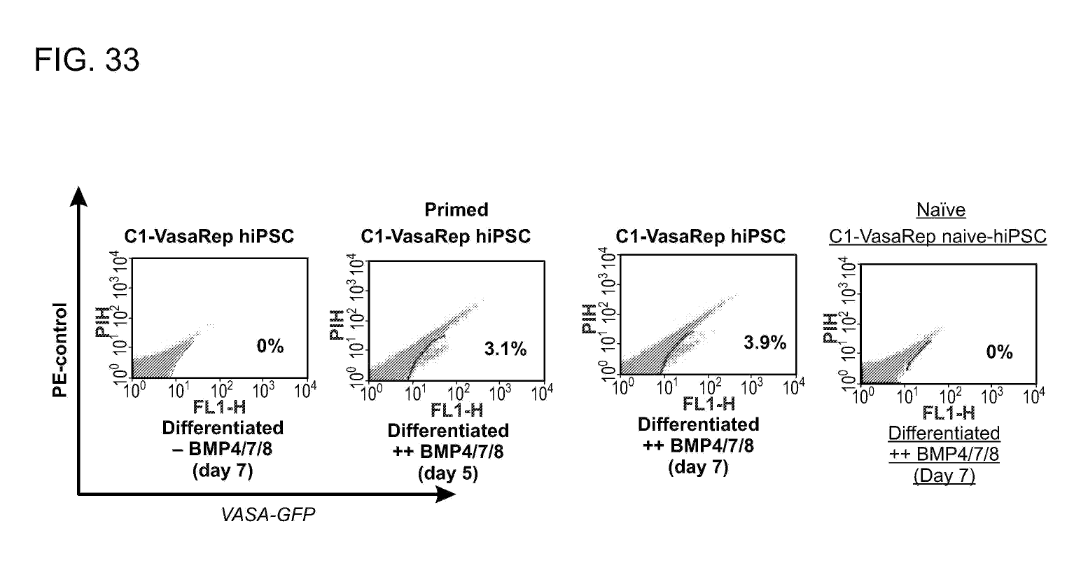

Further work investigated whether naive-hESCs share defining features with mESCs and thus would constitute a pluripotent state that is distinct from the previously described hESCs. ICM derived mouse ESCs are stabilized upon inhibition of the ERK1/2 pathway in contrast to hESCs and mEpiSCs that are induced to differentiate by ERK inhibition (5, 26). Consistent with previously described observations, genetically unmodified conventional hESCs could not be propagated in the presence of ERK1/2 specific inhibitor. Similarly to NOD mESCs, the stability of naive-hESCs was dependent on the continuous presence of ERK1 inhibition (FIG. 11A-B) (11). Also, the naive-hESCs depended on LIF signaling, displayed high levels of pStat3 (FIG. 11C) and readily differentiated when exposed to JAK inhibitor (JAKi) that blocks Stat3 phosphorylation. This behavior is similar to mESCs and contrasts with conventional hESCs, which were resistant to the JAK inhibition (FIG. 11A). Consistent with this observation, naive-hESCs that were stably transfected with a dominant-negative Stat3 encoding transgenes rapidly differentiated and could not be maintained, while cells transgenic for a constitutively active Stat3 mutant (Stat3-CA) could be propagated in the absence of exogenous LIF (FIGS. 11D and 32). Notably, naive-hESC/hiPSC required exogenous LIF as well as PD/CH/FK even when grown on feeder cells (FIG. 11D). In addition, hESC and mEpiSCs rapidly differentiated upon inhibition of the TGFβ/Activin signaling pathway by SB431552 or A83-01, while mESCs and naive-hESC/hiPSC maintained their pluripotent state in response to SB431552 or A83-01 but differentiated upon addition of recombinant TGFβ (FIG. 11A). Finally, addition of BMP4 growth factor or inhibition of bFGF signaling by two different FGF4 receptor inhibitors (PD173074 and SU5401) resulted in the differentiation of hESCs and mEpiSCs, but not of naive-hESCs/hiPSCs or mESCs (FIG. 11A).

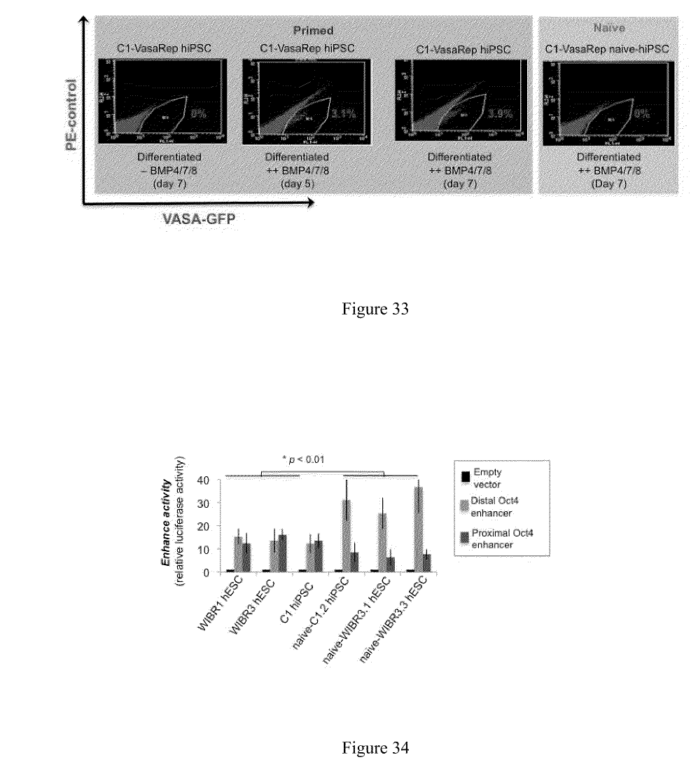

It has been shown that mEpiSCs as well as hESCs are primed for differentiation into primordial germ cell (PGC) like cells and readily activate germ cell markers such as DAZL, PRDM1 (also known as BLIMP1) and VASA upon exposure to BMP4. This is in contrast to mESCs, which are inefficient in activating these markers and require prior embryoid body (EB) formation (7, 17, 27). We tested whether the naive-ESCs would resist activation of PGC markers similar to mESCs (3). FIG. 11E shows that the VASA, BLIMP1 and DAZL were readily activated in hESCs upon culture in BMP4, in contrast to naive-hESCs, which showed no up-regulation of these markers. Similar results were obtained when utilizing a VASA-EGFP reporter transgene to measure efficiency of early PGC differentiation (FIG. 33). In summary, the results shown in FIG. 11 demonstrate that mESCs and naive-hESCs have comparable biological characteristics and depend on similar signaling pathways. However, these pathways are different from those operating in hESCs and mEpiSCs suggesting that the two states of pluripotency can be controlled, at least in part, by similar mechanisms in the human and mouse systems.

Epigenetic Reversion and Maintenance the Pre-X Inactivation State in Female Naïve hESCs

X chromosome inactivation represents an important epigenetic difference between the two states of pluripotency: while female mESCs are in a pre-X inactivation state with two active X chromosomes (XaXa), mEpiSCs and most if not all hESCs have already undergone X inactivation (XiXa) (10, 28). Recently we have demonstrated that environmental conditions such as oxidative stress induce precocious and irreversible X inactivation during early stages of hESC derivation (28). In contrast to the human system, X inactivation is reversible in mouse cells as reprogramming of XiXa somatic cells by nuclear transfer or by in vitro transduction of transcription factors or the conversion of EpiSCs into ESCs has been shown to reactivate the silent X chromosome (10, 29-31). It was of interest, therefore, to test whether the conversion of hESCs into naive-hESCs would reactivate the inactive X chromosome. FIG. 11F shows that WIBR3 hESCs grown in conventional bFGF/serum containing hESC medium and in atmospheric oxygen concentration (20% pO2) or exposed to PD/CH/FK/LIF for 72 hours expressed an XIST cloud in all cells, which is indicative of X inactivation. However, no XIST clouds were seen in all 8 independently derived naive-WIBR3 hESCs and naïve-hiPSCs tested (FIGS. 11F and 27A). Changes in the methylation of the XIST promoter region further support this conclusion (FIG. 27D). When naïve-hESCs were transferred to bFGF/serum-containing hESC growth conditions, the cells adapted the flattened morphology of conventional hESCs. These reverted cells, designated as primed pluripotent cell lines, had initiated X-inactivation as evident by the acquisition of XIST clouds and changes in XIST promoter methylation (e.g. pri-WIBR3.2 in FIGS. 11F and 27D). Overall these results indicate that, similar to mouse cells, X inactivation is reversible and sustainable in naïve-human stem cells after epigenetic reversion of the primed pluripotent state.

Naïve Human Pluripotent Cells are Transcriptionally Similar to Mouse ESCs.

To define molecular signatures of the naive-hESCs, their global gene expression patterns were compared with those of hESCs, mESCs and mEpiSCs. FIG. 12A shows that four independently derived naive hESC lines and two naive-hiPSC lines cluster together and are different from a large number of hESCs and hiPSCs including hESCs grown in mTESR1 defined culture conditions, and Nanog over-expressing hESCs which can grow in a feeder independent manner (32, 33). Notably, human iPSC grown in conventional bFGF/serum containing medium exposed to PD/CH/FK/LIF for 48 hours (31-48h condition in FIG. 11A) did not cluster in their expression profile with naïve-hESC/hiPSCs and highlighting. This highlights that the differential clustering observed does not simply result from the growth conditions applied. Gene array analysis and confirmation by RT-PCR showed that Oct3/4 and Sox2 were expressed at equivalent levels in hESCs/hiPSCs and naïve-hESCs/hiPSCs, while transcripts associated with naïve mESCs such as Klf4, Klf2, Gbx2, Lin28 and SOCS3 were significantly upregulated in naive-hESCs/hiPSCs (FIGS. 12B and 12C). In contrast, transcripts associated with genes expressed in the epiblast and early germ layers as well as in hESCs (3, 4) were significantly downregulated in the naive-hESC/hiPSCs. This set of genes included Otx2, Sox17 Cer1, Foxa2, Zic1, Lhx2 and XIST (FIGS. 12B and 12C). These expression patterns are consistent with previously described differences in gene expression between mESCs and mEpiSCs (3, 4). Finally, FACS analysis showed that hESCs and mouse EpiSCs had initiated surface expression of MHC class I proteins, which are normally expressed on somatic cells (34) in contrast to mESCs and naive-hESC/hiPSC which had residual or no surface expression consistent with a more immature phenotype (FIG. 12D).

An unbiased cross-species hierarchical clustering was performed to assess whether the naïve and primed state of pluripotency in human cells globally corresponded to those characterized in mouse cells. For this, 24 different pluripotent stem cell lines were compared (FIG. 12E), including hESCs/hiPSCs; naïve-hESC/hiPSCs; 129 mESCs and EpiSCs; NOD mESCs; NOD mEpiSC-like cells which were generated from NOD mESCs and miPSCs which were grown in bFGF/Activin after withdrawal of exogenous inhibitors that stabilize their naïve pluripotency (11). Cross-species gene expression analysis on 9,949 mouse-human orthologous genes in the gene expression datasets clustered the samples into two main groups as indicated by the top bifurcation in FIG. 12E, representing two distinct pluripotent states. All naive-hESCs/hiPSCs clustered with mESCs and miPSCs independent of the genetic background, species differences or growth conditions (FIG. 12E). Notably, hESCs and hiPSCs clustered with mouse EpiSCs and NOD EpiSC-like cells and marked anti-correlation was apparent between the naive-hiPSCs/ESCs and the hESC/hiPSCs as indicated by the blue blocks in the correlation matrix (FIG. 12E). Boostrapping-based method (pvclust (35)) supported the reliability of the clustering tree (FIG. 12e). Clusters were invariant when also considering the top 100 and 1000 differentially expressed genes (data not shown). Finally, we measured the activity of the distal and proximal enhancer regions of Oct4 genes that are reciprocally regulated in the pre- and post-implantation mouse embryo (3) as well as in mESCs and mEpiSCs. To compare this regulatory activity by reporter assays, naive-hESC/hiPSCs were transfected with a luciferase reporter construct under the control of either the human distal or the proximal enhancer sequences that control expression of the Oct4 gene, which are highly conserved between mouse and humans (36). Predominant utilization of the Oct4 distal enhancer was observed in naive-hESCs/hiPSCs (FIG. 34), indicating that the gene expression network active in naive-hESCs enhances utilization of the distal Oct4 enhancer as typically observed in mESCs. These findings are consistent with the notion that naive-ESCs have global transcriptional characteristics that are more similar to mESCs than to mEpiSCs or to conventional hESCs.

Discussion

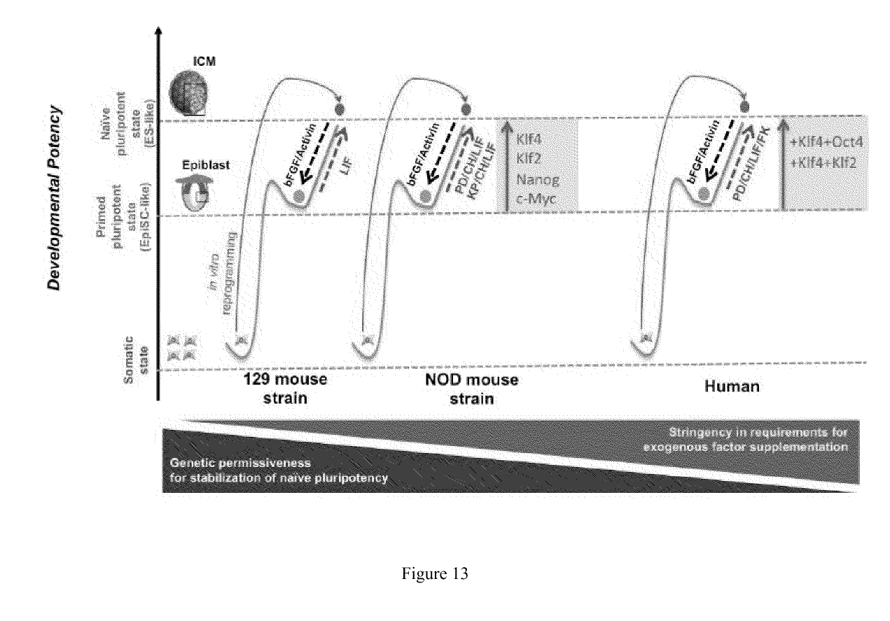

These results demonstrate that naive-hESC and iPSCs are distinct from conventional hESCs/hiPSCs and mEpiSCs, and closely resemble the naïve pluripotent state of ICM-derived mESCs (5, 11) by numerous molecular and biological criteria including growth properties, signaling pathway dependency, state of X chromosome inactivation and transcriptional characteristics. These findings support the notion that distinct states of pluripotency can be specified by defined culture conditions but that genetic background, as well as species differences, determines the requirements and threshold for exogenous factors that establish and maintain the naïve pluripotent state. As schematically shown in FIG. 13, the 129 mouse strain is the most “permissive” genetic background as the LIF/Stat3 signaling pathway is sufficient to stabilize the naïve state of ICM-derived mESCs in the presence of feeder cells, whereas the NOD genetic background requires constitutive expression of Klf4 or c-Myc or simultaneous enhancement of Wnt signaling and inhibition of the ERK1/2 pathway to stabilize the ICM-derived ESCs in the naïve pluripotent state (11, 13). Human cells seem to be the least permissive as naive-hESCs are obtained at relatively low efficiencies and require perturbation of additional molecular pathways in order to achieve stochastic epigenetic reversion and stabilization of the naïve state of pluripotency (FIGS. 13 and 30) (37). In the absence of such exogenous culture and transcription factors, naive-hESCs adapt an EpiSC-like or primed pluripotent state in vitro that is stabilized by bFGF/Activin signaling (FIG. 13). It would be important to delineate whether the latter effect underlies the relative increased heterogeneity in gene expression and differentiation characteristics observed between different conventional hESCs and hiPSCs (FIG. 12A) (38). Moreover, the clonal relationship demonstrated by southern blot analysis between naïve and primed C1 hiPSC lines (FIG. 27) demonstrates that the conversions between the primed and naive pluripotent states are due to inefficient induction of cells to successfully convert from one state to another, rather than due to selection for pre-existing cells continuously present in hESC polyclonal in vitro cultures.

These findings further support previous observations implicating regulation of the KLF transcriptional circuitry in establishing and maintaining naïve pluripotency (10, 11, 39, 40). It will be of interest to define the genetic determinants underlying the different requirements for propagation of the naïve pluripotent state in different species, and whether differences in regulatory regions of KLF transcription factor genes underlie such differences and whether they reflect difference in early development. Moreover, it is possible that the origin of pluripotent cells explanted in vitro for propagation may influence the requirement for exogenous factors to stabilize naïve pluripotency. This hypothesis is supported by the observation that NOD derived iPSCs and ESCs required exogenous supplementation of PD/CH or KP/CH in addition to LIF, while NOD germ line stem (GS) cells remained stable in LIF alone (41). Therefore, it is of importance to re-examine growth requirements and properties for human embryonic pluripotent germ cell lines, possibly derived via in vitro differentiation of primed-hESCs with BMP4 and subsequent reprogramming into EG cell (7, 17).

It should be emphasized that undefined differences may exist between mESCs and the naïve human pluripotent cells as described in this work. Also, further optimization of growth conditions need to be devised to enhance the stability of naïve human pluripotency and to permanently stabilize this novel pluripotent state in the absence of genetic manipulations and whether such conditions can be applicable for other species (42). Notably, shielding Activin/Nodal signaling or FGF4-receptor auto-phosphorylation can further enhance conversion into naïve pluripotency (data not shown and (8)). The naïve-C1 hiPSC lines described in this paper likely constitute a useful system to conduct screens for identifying new pathways that stabilize naïve human pluripotency independently of exogenous transgenes. Finally, the definition and characterization of a novel naïve pluripotent state in human cells may expand the capabilities for utilizing human ESCs and iPSCs in regenerative medicine and disease modeling both in vitro and in vivo (43, 44).

### REFERENCES

1. Evans M J & Kaufman M H (1981) Establishment in culture of pluripotential cells from mouse embryos. Nature 292(5819):154-156.
2. Jaenisch R & Young R (2008) Stem cells, the molecular circuitry of pluripotency and nuclear reprogramming. Cell 132(4):567-582.
3. Tesar P J, et al. (2007) New cell lines from mouse epiblast share defining features with human embryonic stem cells. Nature 448(7150):196-199.
4. Brons I G, et al. (2007) Derivation of pluripotent epiblast stem cells from mammalian embryos. Nature 448(7150):191-195.
5. Nichols J & Smith A (2009) Naive and primed pluripotent states. Cell Stem Cell 4(6):487-492.
6. Rossant J (2008) Stem cells and early lineage development. Cell 132(4):527-531.
7. Hayashi K & Surani M A (2009) Self-renewing epiblast stem cells exhibit continual delineation of germ cells with epigenetic reprogramming in vitro. Development 136(21):3549-3556.
8. Greber B, et al. (onserved and divergent roles of FGF signaling in mouse epiblast stem cells and human embryonic stem cells. Cell Stem Cell 6(3):215-226.
9. Bao 5, et al. (2009) Epigenetic reversion of post-implantation epiblast to pluripotent embryonic stem cells. Nature 461(7268):1292-1295.
10. Guo G, et al. (2009) Klf4 reverts developmentally programmed restriction of ground state pluripotency. Development 136(7):1063-1069.
11. Hanna J, et al. (2009) Metastable pluripotent states in NOD-mouse-derived ESCs. Cell Stem Cell 4(6):513-524.
12. Buehr M, et al. (2008) Capture of authentic embryonic stem cells from rat blastocysts. Cell 135(7):1287-1298.
13. Nichols J, et al. (2009) Validated germline-competent embryonic stem cell lines from nonobese diabetic mice. Nat Med 15(7):814-818.
14. Thomson J A, et al. (1998) Embryonic stem cell lines derived from human blastocysts. Science 282(5391):1145-1147.
15. Xu R H, et al. (2008) NANOG is a direct target of TGFbeta/activin-mediated SMAD signaling in human ESCs. Cell Stem Cell 3(2):196-206.
16. Silva S S, Rowntree R K, Mekhoubad S, & Lee J T (2008) X-chromosome inactivation and epigenetic fluidity in human embryonic stem cells. Proc Natl Acad Sci USA 105(12):4820-4825.
17. Kee K, Angeles V T, Flores M, Nguyen H N, & Reijo Pera R A (2009) Human DAZL, DAZ and BOULE genes modulate primordial germ-cell and haploid gamete formation. Nature 462(7270):222-225.
18. Lovell-Badge R (2007) Many ways to pluripotency. Nat Biotechnol 25(10):1114-1116.
19. Hockemeyer D, et al. (2008) A drug-inducible system for direct reprogramming of human somatic cells to pluripotency. Cell Stem Cell 3(3):346-353.
20. Impey S, et al. (2004) Defining the CREB regulon: a genome-wide analysis of transcription factor regulatory regions. Cell 119(7):1041-1054.
21. Shi Y, et al. (2008) Induction of pluripotent stem cells from mouse embryonic fibroblasts by Oct4 and Klf4 with small-molecule compounds. Cell Stem Cell 3(5):568-574.
22. Ying Q L, et al. (2008) The ground state of embryonic stem cell self-renewal. Nature 453(7194):519-523.
23. Ichida J K, et al. (2009) A small-molecule inhibitor of tgf-Beta signaling replaces sox2 in reprogramming by inducing nanog. Cell Stem Cell 5(5):491-503.
24. Shamblott M J, et al. (1998) Derivation of pluripotent stem cells from cultured human primordial germ cells. Proc Natl Acad Sci USA 95(23):13726-13731.
25. Delghandi M P, Johannessen M, & Moens U (2005) The cAMP signalling pathway activates CREB through PKA, p38 and MSK1 in NIH 3T3 cells. Cell Signal 17(11):1343-1351.
26. Nichols J, Silva J, Roode M, & Smith A (2009) Suppression of Erk signalling promotes ground state pluripotency in the mouse embryo. Development 136(19):3215-3222.
27. Geijsen N, et al. (2004) Derivation of embryonic germ cells and male gametes from embryonic stem cells. Nature 427(6970):148-154.
28. Lengner C J (2010) Derivation of pre-x inactivation human embryonic stem cell line in physiological oxygen conditions. Cell.
29. Eggan K, et al. (2000) X-Chromosome inactivation in cloned mouse embryos. Science 290(5496):1578-1581.
30. Maherali N, et al. (2007) Directly reprogrammed fibroblasts show global epigenetic remodeling and widespread tissue contribution. Cell Stem Cell 1(1):55-70.
31. Takahashi K & Yamanaka S (2006) Induction of pluripotent stem cells from mouse embryonic and adult fibroblast cultures by defined factors. Cell 126(4):663-676.
32. Darr H, Mayshar Y, & Benvenisty N (2006) Overexpression of NANOG in human ES cells enables feeder-free growth while inducing primitive ectoderm features. Development 133(6):1193-1201.
33, Ludwig T E, et al. (2006) Feeder-independent culture of human embryonic stem cells. Nat Methods 3(8):637-646.
34. Drukker M, et al. (2002) Characterization of the expression of MHC proteins in human embryonic stem cells. Proc Natl Acad Sci USA 99(15):9864-9869.
35. Suzuki R & Shimodaira H (2006) Pvclust: an R package for assessing the uncertainty in hierarchical clustering. Bioinformatics 22(12):1540-1542.
36. Nordhoff V, et al. (2001) Comparative analysis of human, bovine, and murine Oct-4 upstream promoter sequences. Mamm Genome 12(4):309-317.
37. Hanna J, et al. (2009) Direct cell reprogramming is a stochastic process amenable to acceleration. Nature 462(7273):595-601.
38. Osafune K, et al. (2008) Marked differences in differentiation propensity among human embryonic stem cell lines. Nat Biotechnol 26(3):313-315.
39. Hall J, et al. (2009) Oct4 and LIF/Stat3 additively induce Kruppel factors to sustain embryonic stem cell self-renewal. Cell Stem Cell 5(6):597-609.
40. Jiang J, et al. (2008) A core Klf circuitry regulates self-renewal of embryonic stem cells. Nat Cell Biol 10(3):353-360.
41. Ohta H, et al. (2009) Male germline and embryonic stem cell lines from NOD mice: efficient derivation of GS cells from a nonpermissive strain for ES cell derivation. Biol Reprod 81(6):1147-1153.
42. Cibelli J B, et al. (2002) Parthenogenetic stem cells in nonhuman primates. Science 295(5556):819.
43. Hanna J, et al. (2007) Treatment of sickle cell anemia mouse model with iPS cells generated from autologous skin. Science 318(5858):1920-1923.
44. Saha K & Jaenisch R (2009) Technical challenges in using human induced pluripotent stem cells to model disease. Cell Stem Cell 5(6):584-595.
45. Hanna J, et al. (2008) Direct reprogramming of terminally differentiated mature B lymphocytes to pluripotency. Cell 133(2):250-264,
46. Niwa H, Ogawa K, Shimosato D, & Adachi K (2009) A parallel circuit of LIF signalling pathways maintains pluripotency of mouse ES cells. Nature 460(7251):118-122.
47. Hubbell E, Liu W M, & Mei R (2002) Robust estimators for expression analysis. Bioinformatics 18(12):1585-1592.
48. Smyth O K & Speed T (2003) Normalization of cDNA microarray data. Methods 31(4):265-273.
49. Liao B Y & Zhang J (2006) Evolutionary conservation of expression profiles between human and mouse orthologous genes. Mol Biol Evol 23(3):530-540.
50. Soldner F, et al. (2009) Parkinson's disease patient-derived induced pluripotent stem cells free of viral reprogramming factors. Cell 136(5):964-977.
51. Yu J, et al. (2007) Induced pluripotent stem cell lines derived from human somatic cells. Science 318(5858):1917-1920.

1. A non-obese diabetic (NOD) embryonic stem (ES) cell.

2.-18. (canceled)

19. A method of producing a chimeric mouse that comprises cells derived from a NOD mouse, the method comprising steps of:
(a) providing a NOD ES cell;
(b) introducing the NOD ES cell into a mouse blastocyst;
(c) transferring the blastocyst to a pseudopregnant female mouse; and
(d) maintaining the female mouse under conditions suitable for production of live offspring.

20.-22. (canceled)

23. A vertebrate cell isolated from the ICM of a vertebrate blastocyst or from the epiblast of a vertebrate embryo, wherein the vertebrate cell has been caused to express Klf4 or c-Myc.

24. (canceled)

25. (canceled)

26. (canceled)

27. A composition comprising: (a) a vertebrate cell isolated from the ICM of a vertebrate blastocyst or isolated from the epiblast of a vertebrate embryo; and (b) a compound that replaces Klf4 or c-Myc expression in generating iPS cells.

28.-45. (canceled)

46. A method of deriving a vertebrate ES cell, the method comprising: (a) providing a vertebrate cell isolated from the ICM of a vertebrate blastocyst or isolated from the epiblast of a vertebrate embryo; (b) causing the cell to persistently express Klf4 or c-Myc or culturing the cell in medium that contains a compound that replaces Klf4 or c-Myc expression in generating iPS cells; and (c) maintaining the cell in culture under conditions suitable and a time sufficient to produce a vertebrate ES cell.

47.-57. (canceled)

58. A method of culturing a vertebrate ES cell, the method comprising: (a) providing a vertebrate ES cell; and (b) culturing the cell in medium that contains a compound that replaces Klf4 or c-Myc expression in generating iPS cells.

59.-66. (canceled)

67. A composition comprising an induced pluripotent stem (iPS) cell and a compound that replaces Klf4 or c-Myc expression in generating iPS cells, wherein said iPS cell is unstable in the absence of the compound.

68.-90. (canceled)

91. A method of deriving an iPS cell from a somatic cell of a NOD mouse comprising steps of:
(a) providing a somatic cell obtained from a NOD mouse;
(b) causing the cell to express a set of reprogramming factors or contacting the cell with a set of reprogramming compounds sufficient to induce the cell to become reprogrammed to a pluripotent state; and
(c) causing the cell to persistently express Klf4 or c-Myc or culturing the cell in medium that contains a compound that replaces Klf4 or c-Myc expression in generating iPS cells.

92. A method of producing a chimeric mouse that comprises cells derived from a NOD mouse, the method comprising steps of:
(a) providing an iPS cell derived from a somatic cell of a NOD mouse;
(b) introducing the iPS cell into a mouse blastocyst;
(c) transferring the blastocyst to a pseudopregnant female mouse; and
(d) maintaining the female mouse under conditions suitable for production of live offspring.

93. (canceled)

94. A method of modifying the pluripotency state of a vertebrate cell to a more naïve state, the method comprising: (a) providing a pluripotent vertebrate cell; (b) causing the cell to express Klf4 and/or Klf2; (c) culturing the cell in the presence of a GSK3 inhibitor, a mitogen-activated protein kinase pathway inhibitor, and an activator of the Stat3 pathway; and (d) maintaining the cell in culture under conditions suitable and a time sufficient to produce a vertebrate cell having a resulting pluripotency state which is more naïve than the pluripotency state of the vertebrate cell of step (a).

95.-130. (canceled)

131. A pluripotent vertebrate cell which has been caused to upregulate expression of KLf4 and/or Klf2 and in which the GSK3 pathway has been inhibited, the mitogen-activated protein kinase pathway has been inhibited, and the Stat3 pathway has been activated.

132. (canceled)

133. (canceled)

134. (canceled)

135. A method of culturing a vertebrate cell, the method comprising: (a) providing a pluripotent vertebrate cell; (b) causing the cell to upregulate expression of Klf4 and/or Klf2; (c) culturing the cell in the presence of a GSK3 inhibitor, a mitogen-activated protein kinase pathway inhibitor, and an activator of the Stat3 pathway.

136.-157. (canceled)
